SEQUENCE LISTING (87 sequences)

SEQ ID NO: 1 - DNA, 181 bp, Lentivirus sp.
  source 1..181: mol_type = genomic DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 2 - DNA, 141 bp, Adeno-associated virus
  source 1..141: mol_type = genomic DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 3 - DNA, 584 bp, Cytomegalovirus aotinebeta1
  source 1..584: mol_type = genomic DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcg

SEQ ID NO: 4 - DNA, 1151 bp, Homo sapiens
  source 1..1151: mol_type = genomic DNA
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgcgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcag

SEQ ID NO: 5 - DNA, 191 bp, unidentified
  source 1..191: mol_type = unassigned DNA
gcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 6 - DNA, 1600 bp, synthetic construct
  source 1..1600: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 7 - DNA, 798 bp, Gallus sp.
  source 1..798: mol_type = genomic DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 8 - DNA, 227 bp, unidentified
  source 1..227: mol_type = unassigned DNA
tgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaac

SEQ ID NO: 9 - DNA, 381 bp, Mus sp.
  source 1..381: mol_type = genomic DNA
aatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtc

SEQ ID NO: 10 - DNA, 48 bp, Pseudomonas aeruginosa
  source 1..48: mol_type = genomic DNA
cttcgagaccgtgacctacatgctcgaagggcgtatgcgccacgaaga

SEQ ID NO: 11 - DNA, 112 bp, unidentified
  source 1..112: mol_type = unassigned DNA
ttccgacgcagtttccgacgagtacgaggacgaggacagacgtgcctaccggcaaggtcaagtggttcaacagcgagaagggcttcggctttctctcccgcgacgacggcgg

SEQ ID NO: 12 - DNA, 39 bp, Streptomyces bacteriophage phiC31
  source 1..39: mol_type = genomic DNA
ccccaactggggtaacctttgagttctctcagttggggg

SEQ ID NO: 13 - DNA, 38 bp, unidentified
  source 1..38: mol_type = unassigned DNA
ggcttgtcgacgacggcggtctccgtcgtcaggatcat

SEQ ID NO: 14 - DNA, 60 bp, unidentified
  source 1..60: mol_type = unassigned DNA
attctcatatccatcttgagtcttctttctcgcaagacaacacgaaatagacacagtctc

SEQ ID NO: 15 - DNA, 112 bp, unidentified
  source 1..112: mol_type = unassigned DNA
tagtgacgtctgtccgcgcagtgatcgagggagtgtgtgctttgccgactggcaaggtcaagccggtctgctaggcacagagagccggtacagtcctccccatgcaacccaa

SEQ ID NO: 16 - DNA, 34 bp, unidentified
  source 1..34: mol_type = unassigned DNA
gtgccagggcgtgcccttgggctccccgggcgcg

SEQ ID NO: 17 - DNA, 52 bp, unidentified
  source 1..52: mol_type = unassigned DNA
gtggtttgtctggtcaaccaccgcggtctcagtggtgtacggtacaaaccca

SEQ ID NO: 18 - DNA, 589 bp, Woodchuck hepatitis virus
  source 1..589: mol_type = genomic DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 19 - DNA, 122 bp, Simian virus 40
  source 1..122: mol_type = genomic DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 20 - DNA, 225 bp, Bos sp.
  source 1..225: mol_type = genomic DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 21 - DNA, 395 bp, Homo sapiens
  source 1..395: mol_type = genomic DNA
gctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtatttta

SEQ ID NO: 22 - DNA, 49 bp, Human Alphaherpesvirus 1
  source 1..49: mol_type = genomic DNA
cggcaataaaaagacagaataaaacgcacgggtgttgggtcgtttgttc

SEQ ID NO: 23 - DNA, 651 bp, synthetic construct
  source 1..651: mol_type = other DNA
atggcttctacgccatttaaattccaattgaaaggcactatcaacggcaaatcttttactgtggaaggggagggggagggtaactcacatgaaggctcccataaaggaaaatatgtctgcacatccgggaagctccctatgtcttgggcagctctgggtacctcattcgggtacggcatgaaatactacaccaaatatccgagtggattgaagaattggttccacgaggtaatgccagagggcttcacatacgaccgccacatccaatataaaggagatggctctatacatgctaagcatcaacacttcatgaagaatggtacttatcataacattgttgaattcacaggccaggatttcaaagaaaactctccggtacttaccggggacatgaatgtaagcttgccgaacgaagttcagcatatacccagagatgacggggtagaatgcccggtcactctgctgtatcccttgttgtcagacaaaagtaaatgtgttgaagcacaccagaacacaatatgtaaaccgctccataatcagcccgcgcctgatgtcccataccattggatacgcaagcagtatacacaaagtaaagatgacacggaggaacgggatcatatatgtcagtccgagacactggaggcacacctc

SEQ ID NO: 24 - protein, 217 aa, synthetic construct
MASTPFKFQLKGTINGKSFTVEGEGEGNSHEGSHKGKYVCTSGKLPMSWAALGTSFGYGMKYYTKYPSGLKNWFHEVMPEGFTYDRHIQYKGDGSIHAKHQHFMKNGTYHNIVEFTGQDFKENSPVLTGDMNVSLPNEVQHIPRDDGVECPVTLLYPLLSDKSKCVEAHQNTICKPLHNQPAPDVPYHWIRKQYTQSKDDTEERDHICQSETLEAHL

SEQ ID NO: 25 - protein, 229 aa, synthetic construct
MDSTEAVIKEFMRFKVHMEGSMNGHEFEIEGEGEGRPYEGTQTAKLRVTKGGPLPFSWDILSPQFMYGSRAFTKHPADIPDYWKQSFPEGFKWERVMNFEDGGAVSVAQDTSLEDGTLIYKVKLRGTNFPPDGPVMQKKTMGWEASTERLYPEDVVLKGDIKMALRLKDGGRYLADFKTTYRAKKPVQMPGAFNIDRKLDITSHNEDYTVVEQYERSVARHSTGGSGGS

SEQ ID NO: 26 - protein, 231 aa, synthetic construct
MAQSKHGLTKEMTMKYRMEGCVDGHKFVITGEGIGYPFKGKQAINLCVVEGGPLPFAEDILSAAFNYGNRVFTEYPQDIVDYFKNSCPAGYTWDRSFLFEDGAVCICNADITVSVEENCMYHESKFYGVNFPADGPVMKKMTDNWEPSCEKIIPVPKQGILKGDVSMYLLLKDGGRLRCQFDTVYKAKSVPRKMPDWHFIQHKLTREDRSDAKNQKWHLTEHAIASGSALP

SEQ ID NO: 27 - protein, 236 aa, synthetic construct
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYK

SEQ ID NO: 28 - protein, 239 aa, synthetic construct
MVSKGEELFTGVVPILVELDGDVNGHKFSVRGEGEGDATNGKLTLKFICTTGKLPVPWPTLVTTFGYGVACFSRYPDHMKQHDFFKSAMPEGYVQERTISFKDDGTYKTRAEVKFEGDTLVNRIELKGIDFKEDGNILGHKLEYNFNSHYVYITADKQKNCIKANFKIRHNVEDGSVQLADHYQQNTPIGDGPVLLPDNHYLSHQSKLSKDPNEKRDHMVLLEFVTAAGITHGMDELYK

SEQ ID NO: 29 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 30 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFNWERVMNFEDGGVVTVTQDSSLQDGEFIYKVKLRGTNFPSDGPVMQCRTMGWEASTERMYPEDGALKGEIKQRLKLKDGGHYDAEVKTTYKAKKPVQLPGAYNVDIKLDILSHNEDYTIVEQYERAEGRHSTGGMDELYK

SEQ ID NO: 31 - protein, 237 aa, synthetic construct
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLN

SEQ ID NO: 32 - protein, 239 aa, synthetic construct
MVSKGEELFTGVVPILVELDGDVNGHKFSVSGEGEGDATYGKLTLKLICTTGKLPVPWPTLVTTLGYGLQCFARYPDHMKQHDFFKSAMPEGYVQERTIFFKDDGNYKTRAEVKFEGDTLVNRIELKGIDFKEDGNILGHKLEYNYNSHNVYITADKQKNGIKANFKIRHNIEDGGVQLADHYQQNTPIGDGPVLLPDNHYLSYQSALSKDPNEKRDHMVLLEFVTAAGITLGMDELYK

SEQ ID NO: 33 - protein, 239 aa, synthetic construct
MVSKGEELFTGVVPILVELDGDVNGHKFSVSGEGEGDATYGKLTLKFICTTGKLPVPWPTLVTTLTWGVQCFARYPDHMKQHDFFKSAMPEGYVQERTIFFKDDGNYKTRAEVKFEGDTLVNRIELKGIDFKEDGNILGHKLEYNAISDNVYITADKQKNGIKANFKIRHNIEDGSVQLADHYQQNTPIGDGPVLLPDNHYLSTQSKLSKDPNEKRDHMVLLEFVTAAGITLGMDELYK

SEQ ID NO: 34 - protein, 310 aa, synthetic construct
MAEGSVARQPDLLTCEHEEIHLAGSIQPHGALLVVSEHDHRVIQASANAAEFLNLGSVLGVPLAEIDGDLLIKILPHLDPTAEGMPVAVRCRIGNPSTEYCGLMHRPPEGGLIIELERAGPSIDLSGTLAPALERIRTAGSLRALCDDTVLLFQQCTGYDRVMVYRFDEQGHGLVFSECHVPGLESYFGNRYPSSTVPQMARQLYVRQRVRVLVDVTYQPVPLEPRLSPLTGRDLDMSGCFLRSMSPCHLQFLKDMGVRATLAVSLVVGGKLWGLVVCHHYLPRFIRFELRAICKRLAERIATRITALES

SEQ ID NO: 35 - DNA, 1578 bp, synthetic construct
  source 1..1578: mol_type = other DNA
atgggccctagcgccttcagctacgtgcgctttagcagcggcaagcaggccaagggcagctctgaacacagacagagagccatgctgggacagtggctggaacagcaccctagcttcacactgagtgatctgcggttcgaggacctgggcagaagcggattcagcggcgagcacctggatcacggcctgggccagctgctggctgctatcgacagcggcgccatcaagagcggagatgtgatcctggtcgaggccgtggacagaatcggcagactggagcctctggaaatgctgcctctgttctccagaatcgtgaaggccggcgtgtccgttatcaccctggaagatggccacgtttacgaccggagctccgtgaacgagacatctctgtttctgctggtggccaaaatccagcaggcccacgagtactctaatagactgtctcgaagaatcaacgcctcttacaccgctcggagagaaaaagctaaagcaggcctgggcatcaagagagagacacctgtgtggctgaccacagacggccaactggtgcctcacgtggcccctcacatcgcccaggcttttcaggactacgccgatggcctgggagagagaaggatttgtagaaagctgcgggagagcggcctggaagaattctccaagaccaacgccaccaccgtgcggagatggctgaagaaccggaccgccattggatattggaacgacatccctgatgtgtaccctcatgtcgtggaccctgctctgttctaccaggtgcagcagcgcctggacgcccctaaggtggacagagccaagcccagcgctcactacctgacaggcctcgtgaagtgcgccgtgtgcggcagaaactacaactacaaacagcggaagcacaccgaccccgctatgctgtgcaccagcagagccagactggccggggaaggctgcagcaatagcaagacataccccgtgatagttctggaccaggtgcggaagctgaccagcctgccgttcctgcaacacgccatggaatccgcctcatctcaggccgacccaagcagccagagactggccgtgatcgacggcgaaatcggcgagctgtccagaaagatcagcgaggccacaaaggccctgctggtgctgggcttcacccctgagatccaggagagcctggaacagctcaagaccgccagagaagccctggaggaagaaagagccaccctgctgctgcctcaggccgagaagctgacaacagctcagctggaggccttcagcaacggcctgctggacgacgagcccatgaagctcaaccacgtgcttcagaccgccggctacagcatggtggtgcaccccgacggctctatcgacgtggatggaaaaagattcgtgtacgagggcgctagccggaaagagaaagtgtacaagctgagactgatcggcgaggataagcagtggagcctgcccatcctgaccccacagatggccacttacaagtccctgttcatggccgctgtgcggctgcctggcgaccccagcgaggaagagctgagacggttcgaggaagccaagcacagcgagcgg

SEQ ID NO: 36 - protein, 526 aa, synthetic construct
MGPSAFSYVRFSSGKQAKGSSEHRQRAMLGQWLEQHPSFTLSDLRFEDLGRSGFSGEHLDHGLGQLLAAIDSGAIKSGDVILVEAVDRIGRLEPLEMLPLFSRIVKAGVSVITLEDGHVYDRSSVNETSLFLLVAKIQQAHEYSNRLSRRINASYTARREKAKAGLGIKRETPVWLTTDGQLVPHVAPHIAQAFQDYADGLGERRICRKLRESGLEEFSKTNATTVRRWLKNRTAIGYWNDIPDVYPHVVDPALFYQVQQRLDAPKVDRAKPSAHYLTGLVKCAVCGRNYNYKQRKHTDPAMLCTSRARLAGEGCSNSKTYPVIVLDQVRKLTSLPFLQHAMESASSQADPSSQRLAVIDGEIGELSRKISEATKALLVLGFTPEIQESLEQLKTAREALEEERATLLLPQAEKLTTAQLEAFSNGLLDDEPMKLNHVLQTAGYSMVVHPDGSIDVDGKRFVYEGASRKEKVYKLRLIGEDKQWSLPILTPQMATYKSLFMAAVRLPGDPSEEELRRFEEAKHSER

SEQ ID NO: 37 - protein, 555 aa, synthetic construct
MQPNLRYLACLRLSADSDGSTSIEWQRGVIRHHVSSPHLSGVLVGEAEDTDVSGSLSPFKRPKLGKWLTAKADEFDVIIAAKMDRLTRRSMHFNELLEWAQQNGKFIVCVEEGFDLSTPQGKMMARMTAVFAEAEWDTIQARILNGVQTRLENRSWLVGAPPTGYRIKTVEGGKRKILEIDQDFYPYVEEIFRRIREGQSTHRIARDFNGRSVLTWGDHLRKLKGEEPKGTQWQATIINKFIRSSWVPGLYTYKGEAVLDDQGDPVILPETPLATMDEWTDLVDRIKPAPKPEGATGGSRNSAKSLLSGVAHCGECGAPFTSLMDSGYKRKDGTKVPGHRRYRCSNKFKGGDCKNGSYVRADVLDSWVDQAIRDSIGQEDMYERAGKGPSQARELQETKARLAKLEADYESGKYDGEGQDESYWRMNKNLSAKVAHLAKQEAERANPTFKATGKKYGEVWEAKDQEDRRDFLRTYGVKVFVWGEGADKKDRGYAMNLGDIKTMAEELFPNRDRARFKLVHTHNAPEGYLSKIGIAVGLLKYGHPLEVKLRSPENS

SEQ ID NO: 38 - protein, 604 aa, synthetic construct
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVA

SEQ ID NO: 39 - protein, 500 aa, synthetic construct [sequence omitted]

SEQ ID NO: 40 - protein, 199 aa, synthetic construct
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 41 - DNA, 396 bp, synthetic construct
  source 1..396: mol_type = other DNA
atggcgaagcccctgtcacaggaggagagtaccctgattgaacgggcgacagcgacaataaacagcatcccgattagtgaggattactccgtcgcatcagcagcactgagttctgatggtaggatcttcaccggggtgaacgtctaccacttcacggggggaccatgtgcagagttggtcgttttgggaaccgcagcagcagcggcggctggtaatctgacttgtatagtagccatcggaaatgaaaaccgcggcattctgtccccgtgcgggcgctgcaggcaggttttgctggatctgcatcccgggataaaagctattgtaaaagattcagacggccagccaacggccgttggcatcagggagcttcttccatccggttatgtgtgggagggc

SEQ ID NO: 42 - protein, 132 aa, synthetic construct
MAKPLSQEESTLIERATATINSIPISEDYSVASAALSSDGRIFTGVNVYHFTGGPCAELVVLGTAAAAAAGNLTCIVAIGNENRGILSPCGRCRQVLLDLHPGIKAIVKDSDGQPTAVGIRELLPSGYVWEG

SEQ ID NO: 43 - protein, 341 aa, synthetic construct
MKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKE

SEQ ID NO: 44 - protein, 267 aa, synthetic construct
MGSAIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKARMPDGDDLVVTHGDACLPNIMVENGRFSGFIDCGRLGVADRYQDIALATRDIAEELGGEWADRFLVLYGIAAPDSQRIAFYRLLDEFF

SEQ ID NO: 45 - DNA, 1716 bp, synthetic construct
  source 1..1716: mol_type = other DNA
atgatcagtctgattgcggcgttagcggtagatcgcgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgggtacggacgatcgcgtaacgcgcgtgaagtcggtggatgaagccatcgcggcgtgtggtgacgtaccagaaatcatggtgattggcggcggtcgcgtttatgaacagttcttgccaaaagcgcaaaaactgtatctgagccatatcgacgcagaagtggacggcgacacccatttcccggattacgagccggatgactgggaatcggtattcagcgaattccacgatgctgatgcgctgaactctcacagctattgctttgagattctggagcggcgaagtggatctgagacaccaggcacgtctgagtctgcaactccagagtccggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 46 - protein, 572 aa, synthetic construct
MISLIAALAVDRVIGMENAMPWNLPADLAWFKRNTLNKPVIMGRHTWESIGRPLPGRKNIILSSQPGTDDRVTRVKSVDEAIAACGDVPEIMVIGGGRVYEQFLPKAQKLYLSHIDAEVDGDTHFPDYEPDDWESVFSEFHDADALNSHSYCFEILERRSGSETPGTSESATPESGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSGVDGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 47 - protein, 376 aa, Human Alphaherpesvirus 1
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 48 - protein, 621 aa, Adeno-associated virus
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLAERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRARESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCISEQ

SEQ ID NO: 49 - protein, 735 aa, Adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGSTTMATGSGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQTNSGTLQQSRLLFSQAGPTNMSLQAKNWLPGPCYRQQRLSKQANDNNNSNFPWTAATKYHLNGRDSLVNPGPAMASHKDDEEKFFPMHGTLIFGKQGTNANDADLENVMITDEEEIRTTNPVATEQYGTVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 50 - RNA, 41 nt, synthetic construct
  source 1..41: mol_type = other RNA
ttatcaacttgaaaaagtggcaccgagtcggtgcttttttt

SEQ ID NO: 51 - DNA, 816 bp, Homo sapiens
  source 1..816: mol_type = genomic DNA
tgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacag

SEQ ID NO: 52 - DNA, 826 bp, Homo sapiens
  source 1..826: mol_type = genomic DNA
gattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctct

SEQ ID NO: 53 - DNA, 20 bp, synthetic construct
  source 1..20: mol_type = other DNA
ggggccactagggacaggat

SEQ ID NO: 54 - DNA, 2763 bp, synthetic construct
  source 1..2763: mol_type = other DNA
  misc_difference 2664: note = a, c, t, g, unknown or other
  misc_difference 2667: note = a, c, t, g, unknown or other
  misc_difference 2670: note = a, c, t, g, unknown or other
  misc_difference 2673: note = a, c, t, g, unknown or other
  misc_difference 2676: note = a, c, t, g, unknown or other
  misc_difference 2679: note = a, c, t, g, unknown or other
  misc_difference 2682: note = a, c, t, g, unknown or other
  misc_difference 2685: note = a, c, t, g, unknown or other
  misc_difference 2716: note = a, c, t, g, unknown or other
  misc_difference 2717: note = a, c, t, g, unknown or other
  misc_difference 2719: note = a, c, t, g, unknown or other
  misc_difference 2720: note = a, c, t, g, unknown or other
  misc_difference 2722: note = a, c, t, g, unknown or other
  misc_difference 2723: note = a, c, t, g, unknown or other
  misc_difference 2725: note = a, c, t, g, unknown or other
  misc_difference 2726: note = a, c, t, g, unknown or other
  misc_difference 2728: note = a, c, t, g, unknown or other
  misc_difference 2729: note = a, c, t, g, unknown or other
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcactgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacggaccacgacgccagcgccgcgaccaccaacaccggcgcccaccatcgcgtcgcagcctctgtccctgcgcccagaggcgtgccgaccagcggcgggtggagcagtgcacacgagggggctggacttcgcctgtgatatctacatctgggcgcccttggccgggacttgtggggtccttctcctgtcactggttatcaccctttactgcaagcggggcagaaagaagctgctgtacatcttcaagcagcccttcatgcggcccgtgcagaccacccaggaagaggacggctgctcctgcagattccccgaggaagaagaaggcggctgcgagctgagagtgaagttcagcaggagcgcagacgcccccgcgtaccagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcactccatatgcaagcccttcctccaagaggaagcggagctactaacttcagcctgctgaagcaggctggtgacgtggaggagaaccctggacccatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggacccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtgtcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtcccacgaatgggcctaagatcccgtccatcgccactgggatggtgggggccctcctcttgctgctggtggtggccctggggatcggcctcttcatgggcagcggcgcgaccaactttagcctgctgaaacaggcgggcgatgttgaagaaaacccaggtcctggntcnggntcnggntcnggntcngggccttcaggtagtcgtgacgtcgggagtnnknnknnknnknnktcggctgctttaaggccggtcctagcaaagtga

SEQ ID NO: 55 - DNA, 2763 bp, synthetic construct
  source 1..2763: mol_type = other DNA
  misc_difference 2664: note = a, c, t, g, unknown or other
  misc_difference 2667: note = a, c, t, g, unknown or other
  misc_difference 2670: note = a, c, t, g, unknown or other
  misc_difference 2673: note = a, c, t, g, unknown or other
  misc_difference 2676: note = a, c, t, g, unknown or other
  misc_difference 2679: note = a, c, t, g, unknown or other
  misc_difference 2682: note = a, c, t, g, unknown or other
  misc_difference 2685: note = a, c, t, g, unknown or other
  misc_difference 2716: note = a, c, t, g, unknown or other
  misc_difference 2717: note = a, c, t, g, unknown or other
  misc_difference 2719: note = a, c, t, g, unknown or other
  misc_difference 2720: note = a, c, t, g, unknown or other
  misc_difference 2722: note = a, c, t, g, unknown or other
  misc_difference 2723: note = a, c, t, g, unknown or other
  misc_difference 2725: note = a, c, t, g, unknown or other
  misc_difference 2726: note = a, c, t, g, unknown or other
  misc_difference 2728: note = a, c, t, g, unknown or other
  misc_difference 2729: note = a, c, t, g, unknown or other
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcactgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacggaccacgacgccagcgccgcgaccaccaacaccggcgcccaccatcgcgtcgcagcctctgtccctgcgcccagaggcgtgccgaccagcggcgggtggagcagtgcacacgagggggctggacttcgcctgtgatatctacatctgggcgcccttggccgggacttgtggggtccttctcctgtcactggttatcaccctttactgcgtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcactccatatgcaagcccttcctccaagaggaagcggagctactaacttcagcctgctgaagcaggctggtgacgtggaggagaaccctggacccatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggacccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtgtcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtcccacgaatgggcctaagatcccgtccatcgccactgggatggtgggggccctcctcttgctgctggtggtggccctggggatcggcctcttcatgggcagcggcgcgaccaactttagcctgctgaaacaggcgggcgatgttgaagaaaacccaggtcctggntcnggntcnggntcnggntcngggccttcaggtagtcgtgacgtcgggagtnnknnknnknnknnktcggctgctttaaggccggtcctagcaaagtga

SEQ ID NO: 56 - DNA, 2763 bp, synthetic construct
  source 1..2763: mol_type = other DNA
  misc_difference 2664: note = a, c, t, g, unknown or other
  misc_difference 2667: note = a, c, t, g, unknown or other
  misc_difference 2670: note = a, c, t, g, unknown or other
  misc_difference 2673: note = a, c, t, g, unknown or other
  misc_difference 2676: note = a, c, t, g, unknown or other
  misc_difference 2679: note = a, c, t, g, unknown or other
  misc_difference 2682: note = a, c, t, g, unknown or other
  misc_difference 2685: note = a, c, t, g, unknown or other
  misc_difference 2716: note = a, c, t, g, unknown or other
  misc_difference 2717: note = a, c, t, g, unknown or other
  misc_difference 2719: note = a, c, t, g, unknown or other
  misc_difference 2720: note = a, c, t, g, unknown or other
  misc_difference 2722: note = a, c, t, g, unknown or other
  misc_difference 2723: note = a, c, t, g, unknown or other
  misc_difference 2725: note = a, c, t, g, unknown or other
  misc_difference 2726: note = a, c, t, g, unknown or other
  misc_difference 2728: note = a, c, t, g, unknown or other
  misc_difference 2729: note = a, c, t, g, unknown or other
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcactgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacggaccacgacgccagcgccgcgaccaccaacaccggcgcccaccatcgcgtcgcagcctctgtccctgcgcccagaggcgtgccgaccagcggcgggtggagcagtgcacacgagggggctggacttcgcctgtgatatctacatctgggcgcccttggccgggacttgtggggtccttctcctgtcactggttatcaccctttactgcgtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgctcctgcctatcagcagggtcagaaccagcttttcaatgaactgaacctcggcagacgcgaggagtttgatgtgcttgataagcggcgggggcgggaccctgagatgggtgggaagccaagacgcaagaatccccaagaggggcttttcaacgaactccagaaagataagatggccgaagctttttcagagatcggcatgaagggtgagcgaaggagggggaagggccatgacggtctttttcaaggactgtccacggcaactaaggatactttcgacgcactccatatgcaggcattgccaccacgcggaagcggagctactaacttcagcctgctgaagcaggctggtgacgtggaggagaaccctggacccatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggacccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtgtcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtcccacgaatgggcctaagatcccgtccatcgccactgggatggtgggggccctcctcttgctgctggtggtggccctggggatcggcctcttcatgggcagcggcgcgaccaactttagcctgctgaaacaggcgggcgatgttgaagaaaacccaggtcctggntcnggntcnggntcnggntcngggccttcaggtagtcgtgacgtcgggagtnnknnknnknnknnktcggctgctttaaggccggtcctagcaaagtga

SEQ ID NO: 57 - DNA, 2763 bp, synthetic construct
  source 1..2763: mol_type = other DNA
  misc_difference 2664: note = a, c, t, g, unknown or other
  misc_difference 2667: note = a, c, t, g, unknown or other
  misc_difference 2670: note = a, c, t, g, unknown or other
  misc_difference 2673: note = a, c, t, g, unknown or other
  misc_difference 2676: note = a, c, t, g, unknown or other
  misc_difference 2679: note = a, c, t, g, unknown or other
  misc_difference 2682: note = a, c, t, g, unknown or other
  misc_difference 2685: note = a, c, t, g, unknown or other
  misc_difference 2716: note = a, c, t, g, unknown or other
  misc_difference 2717: note = a, c, t, g, unknown or other
  misc_difference 2719: note = a, c, t, g, unknown or other
  misc_difference 2720: note = a, c, t, g, unknown or other
  misc_difference 2722: note = a, c, t, g, unknown or other
  misc_difference 2723: note = a, c, t, g, unknown or other
  misc_difference 2725: note = a, c, t, g, unknown or other
  misc_difference 2726: note = a, c, t, g, unknown or other
  misc_difference 2728: note = a, c, t, g, unknown or other
  misc_difference 2729: note = a, c, t, g, unknown or other
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcactgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacggaccacgacgccagcgccgcgaccaccaacaccggcgcccaccatcgcgtcgcagcctctgtccctgcgcccagaggcgtgccgaccagcggcgggtggagcagtgcacacgagggggctggacttcgcctgtgatatctacatctgggcgcccttggccgggacttgtggggtccttctcctgtcactggttatcaccctttactgcgtgaggagtaagaggagcaggctcctgcacagtgactaaatgaacatgactccccgccgccccgggcccacccgcaagcattaccagccctaagccccaccacgcgacttcgcagcctaacgctccagagtgaagttcagcaggagcgcagacgcccccgcgtaacagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcactccatatgcaagcccttcctccaagaggaagcggagctactaacttcagcctgctgaagcaggctggtgacgtggaggagaaccctggacccatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggacccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtgtcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtcccacgaatgggcctaagatcccgtccatcgccactgggatggtgggggccctcctcttgctgctggtggtggccctggggatcggcctcttcatgggcagcggcgcgaccaactttagcctgctgaaacaggcgggcgatgttgaagaaaacccaggtcctggntcnggntcnggntcnggntcngggccttcaggtagtcgtgacgtcgggagtnnknnknnknnknnktcggctgctttaaggccggtcctagcaaagtga

SEQ ID NO: 58 - DNA, 3018 bp, synthetic construct
  source 1..3018: mol_type = other DNA
  misc_difference 2919: note = a, c, t, g, unknown or other
  misc_difference 2922: note = a, c, t, g, unknown or other
  misc_difference 2925: note = a, c, t, g, unknown or other
  misc_difference 2928: note = a, c, t, g, unknown or other
  misc_difference 2931: note = a, c, t, g, unknown or other
  misc_difference 2934: note = a, c, t, g, unknown or other
  misc_difference 2937: note = a, c, t, g, unknown or other
  misc_difference 2940: note = a, c, t, g, unknown or other
  misc_difference 2971: note = a, c, t, g, unknown or other
  misc_difference 2972: note = a, c, t, g, unknown or other
  misc_difference 2974: note = a, c, t, g, unknown or other
  misc_difference 2975: note = a, c, t, g, unknown or other
  misc_difference 2977: note = a, c, t, g, unknown or other
  misc_difference 2978: note = a, c, t, g, unknown or other
  misc_difference 2980: note = a, c, t, g, unknown or other
  misc_difference 2981: note = a, c, t, g, unknown or other
  misc_difference 2983: note = a, c, t, g, unknown or other
  misc_difference 2984: note = a, c, t, g, unknown or other
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcactgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacggaccacgacgccagcgccgcgaccaccaacaccggcgcccaccatcgcgtcgcagcctctgtccctgcgcccagaggcgtgccgaccagcggcgggtggagcagtgcacacgagggggctggacttcgcctgtgatatctacatctgggcgcccttggccgggacttgtggggtccttctcctgtcactggttatcaccctttactgcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaagcggagctactaacttcagcctgctgaagcaggctggtgacgtggaggagaaccctggacccatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggacccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtgtcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtcccacgaatgggcctaagatcccgtccatcgccactgggatggtgggggccctcctcttgctgctggtggtggccctggggatcggcctcttcatgggcagcggcgcgaccaactttagcctgctgaaacaggcgggcgatgttgaagaaaacccaggtcctggntcnggntcnggntcnggntcngggccttcaggtagtcgtgacgtcgggagtnnknnknnknnknnktcggctgctttaaggccggtcctagcaaagtga

SEQ ID NO: 59 - DNA, 3009 bp, synthetic construct
  source 1..3009: mol_type = other DNA
  misc_difference 2910: note = a, c, t, g, unknown or other
  misc_difference 2913: note = a, c, t, g, unknown or other
  misc_difference 2916: note = a, c, t, g, unknown or other
  misc_difference 2919: note = a, c, t, g, unknown or other
  misc_difference 2922: note = a, c, t, g, unknown or other
  misc_difference 2925: note = a, c, t, g, unknown or other
  misc_difference 2928: note = a, c, t, g, unknown or other
  misc_difference 2931: note = a, c, t, g, unknown or other
  misc_difference 2962: note = a, c, t, g, unknown or other
  misc_difference 2963: note = a, c, t, g, unknown or other
  misc_difference 2965: note = a, c, t, g, unknown or other
  misc_difference 2966: note = a, c, t, g, unknown or other
  misc_difference 2968: note = a, c, t, g, unknown or other
  misc_difference 2969: note = a, c, t, g, unknown or other
  misc_difference 2971: note = a, c, t, g, unknown or other
  misc_difference 2972: note = a, c, t, g, unknown or other
  misc_difference 2974: note = a, c, t, g, unknown or other
  misc_difference 2975: note = a, c, t, g, unknown or other
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcactgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacggaccacgacgccagcgccgcgaccaccaacaccggcgcccaccatcgcgtcgcagcctctgtccctgcgcccagaggcgtgccgaccagcggcgggtggagcagtgcacacgagggggctggacttcgcctgtgatatctacatctgggcgcccttggccgggacttgtggggtccttctcctgtcactggttatcaccctttactgcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaattgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagtgtcgtaccatgggctgggaggcctccactgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcgacatcaagttggacatcctttcccacaacgaggactacaccatcgtggaacagtacgaacgcgccgagggccgccactccaccggcggcatggacgagctgtacaagggaagcggagctactaacttcagcctgctgaagcaggctggtgacgtggaggagaaccctggacccatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggacccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtgtcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtcccacgaatgggcctaagatcccgtccatcgccactgggatggtgggggccctcctcttgctgctggtggtggccctggggatcggcctcttcatgggcagcggcgcgaccaactttagcctgctgaaacaggcgggcgatgttgaagaaaacccaggtcctggntcnggntcnggntcnggntcngggccttcaggtagtcgtgacgtcgggagtnnknnknnknnknnktcggctgctttaaggccggtcctagcaaagtga

SEQ ID NO: 60 - protein, 386 aa, unidentified
MHGLQDDPDLQALLKGSQLLKVKSSSWRRERFYKLQEDCKTIWQESRKVMRSPESQLFSIEDIQEVRMGHRTEGLEKFARDIPEDRCFSIVFKDQRNTLDLIAPSPADAQHWVQGLRKIIHHSGSMDQRQKLQSRPEPTAPPEESFRSGVETTTPPQKQEPIDKELYPLTSLRSLFGNDPSSQKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCT

SEQ ID NO: 61 - protein, 500 aa, unidentified
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 62 - protein, 534 aa, unidentified
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLL

SEQ ID NO: 63 - protein, 58 aa, unidentified
FNSSINNIHEMEIQLKDALEKNQQWLVYDQQREVYVKGLLAKIFELEKKTETAAHSLP

SEQ ID NO: 64 - protein, 110 aa, unidentified
ALPGNPDHREMGETLPEEVGEYRQPSGGSVPVSPGPPSGLEPTSSSPYGGGSFNSSINNIHEMEIQLKDALEKNQQWLVYDQQREVYVKGLLAKIFELEKKTETAAHSLP

SEQ ID NO: 65 - protein, 52 aa, unidentified
LQSRPEPTAPPEESFRSGVETTTPPQKQEPIDKELYPLTSLRSLFGNDPSSQ

SEQ ID NO: 66 - protein, 116 aa, unidentified
ASNFTQFVLVDNGGTGDVTVAPSNFANGIAEWISSNSRSQAYKVTCSVRQSSAQKRKYTIKVEVPKGAWRSYLNMELTIPIFATNSDCELIVKAMQGLLKDGNPIPSAIAANSGIY

SEQ ID NO: 67 - protein, 129 aa, unidentified
ASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 68 - protein, 123 aa, unidentified
MGSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGR

SEQ ID NO: 69 - protein, 270 aa, unidentified
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAA

SEQ ID NO: 70 - protein, 263 aa, unidentified
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 71 - protein, 352 aa, unidentified [sequence omitted]

SEQ ID NO: 72 - protein, 352 aa, unidentified
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 73 - protein, 366 aa, unidentified
MDFLNSSDQNLTSEELLNRMPSKILVSLTLSGLALMTTTINSLVIAAIIVTRKLHHPANYLICSLAVTDFLVAVLVMPFSIVYIVRESWIMGQVVCDIWLSVDITCCTCSILHLSAIALDRYRAITDAVEYARKRTPKHAGIMITIVWIISVFISMPPLFWRHQGTSRDDECIIKHDHIVSTIYSTFGAFYIPLALILILYYKIYRAAKTLYHKRQASRIAKEEVNGQVLLESGEKSTKSVSTSYVLEKSLSDPSTDFDKIHSTVRSLRSEFKHEKSWRRQKISGTRERKAATTLGLILGAFVICWLPFFVKELVVNVCDKCKISEEMSNFLAWLGYLNSLINPLIYTIFNEDFKKAFQKLVRCRC

SEQ ID NO: 74 - protein, 290 aa, unidentified
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 75 - protein, 288 aa, unidentified
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 76 - protein, 329 aa, unidentified
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 77 - protein, 223 aa, unidentified
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 78 - protein, 238 aa, unidentified
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDKVMSEFNNNFRQQMENYPKNNHTASILDRMQADFKCCGAANYTDWEKIPSMSKNRVPDSCCINVTVGCGINFNEKAIHKEGCVEKIGGWLRKNVLVVAAAALGIAFVEVLGIVFACCLVKSIRSGYEVM

SEQ ID NO: 79 - protein, 236 aa, unidentified
MGVEGCTKCIKYLLFVFNFVFWLAGGVILGVALWLRHDPQTTNLLYLELGDKPAPNTFYVGIYILIAVGAVMMFVGFLGCYGAIQESQCLLGTFFTCLVILFACEVAAGIWGFVNKDQIAKDVKQFYDQALQQAVVDDDANNAKAVVKTFHETLDCCGSSTLTALTTSVLKNNLCPSGSNIISNLFKEDCHQKIDDLFSGKLYLIGIAAIVVAVIMIFEMILSMVLCCGIRNSSVY

SEQ ID NO: 80 - protein, 228 aa, unidentified
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 81 - protein, 511 aa, unidentified
MATGSRTSLLLAFGLLCLPWLQEGSAELAGIGILTVGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 82 - protein, 510 aa, unidentified
MATGSRTSLLLAFGLLCLPWLQEGSASLLMWITQVGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 83 - protein, 511 aa, unidentified
MVPCTLLLLLAAALAPTQTRASIINFEKLGCGASGGGGSGGGGSIQKTPQIQVYSRHPPENGKPNILNCYVTQFHPPHIEIQMLKNGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGCYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEPPPSTVSNMATVAVLVVLGAAIVTGAVVAFVMKMRRRNTGGKGGDYALAPGSQTSDLSLPDCKVMVHDPHSLA

SEQ ID NO: 84 - protein, 556 aa, unidentified [sequence omitted]

SEQ ID NO: 85 - protein, 220 aa, unidentified
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 86 - protein, 288 aa, unidentified
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 87 - DNA, 231 bp, unidentified
  source 1..231: mol_type = unassigned DNA
agggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaa

(19) United States
(12) Patent Application Publication
(10) Pub. No.: US 2025/0313829 A1
(43) Pub. Date: Oct. 9, 2025

(54) COMPOSITIONS COMPRISING CELL LINES AND METHODS OF GENERATING VIRAL PARTICLES USING THE SAME

(71) Applicant: THE REGENTS OF THE UNIVERSITY OF CALIFORNIA, Oakland, CA (US)

(72) Inventors: Neil Q. Tay, San Francisco, CA (US); Michael T. McManus, San Francisco, CA (US)

(73) Assignee: THE REGENTS OF THE UNIVERSITY OF CALIFORNIA, Oakland, CA (US)

(21) Appl. No.: 18/983,241

(22) Filed: Dec. 16, 2024

Related U.S. Application Data
(60) Provisional application No. 63/610,389, filed on Dec. 14, 2023.

(51) Int. Cl. C12N 15/10; C12N 15/86

(52) U.S. Cl. CPC C12N 15/1082; C12N 15/86; C12N 2740/15023; C12N 2740/15043

(57) ABSTRACT

The disclosure provides novel virus packaging elements and cells transduced by such elements to enable large-scale library screens with combinatorial elements that heretofore were impractical due to the presence of recombination between library elements that occurs during conventional lentivirus production and transduction of target cells. The present disclosure overcomes these problems by generating clonal virus packaging cells that each produce a genetically homologous virus.

### CROSS-REFERENCE TO RELATED APPLICATIONS

This application claims the benefit of U.S. provisional application No. 63/610,389, which was filed Dec. 14, 2023, is titled COMPOSITIONS COMPRISING CELL LINES AND METHODS OF GENERATING VIRAL PARTICLES USING THE SAME, and is incorporated herein by reference in its entirety.

### SEQUENCE LISTING

The electronic sequence listing filed herewith, having a filename UCAL-033-US_SL.xml, created on Dec. 16, 2024, and having of file size of 4,575,757 bytes is incorporated herein by reference as if fully set forth.

### FIELD

The disclosure relates to compositions comprising cell lines comprising a transduction targeting element that directs integration and expression of identical viral mRNA or DNA after transiently or stable integration of a transduction targeting element. The disclosure also relates to the preparation of lentiviral and/or cell libraries wherein each lentivirus particle comprises genetically homozygous or identical payload, hence eliminating the possibility of genetic recombination between library elements within a viral library.

### BACKGROUND

The information available from genome sequencing efforts has transformed the nature of biological inquiry and has led to an increased need for tools that enable genome-scale functional studies. Lentiviral vectors are widely used for functional genomic screens, enabling efficient and stable transduction of target cells with libraries of genetic elements.

Unfortunately, designs that rely upon integrating multiple variable sequences, such as combinatorial perturbations or perturbations linked to barcodes may be compromised by unintended consequences of lentiviral packaging. Intermolecular recombination between library elements and integration of multiple perturbations, even at limiting virus dilution, can negatively impact the sensitivity of pooled screens. Recombination can arise from the template-switching of the lentiviral reverse-transcriptase. As the lentivirus capsid normally packages a dimer of RNA genomes, intermolecular recombination can occur in target cells infected by a single virion. The fraction of target cells with recombined integrants depends on the distance between variable sequences and has been measured to exceed 30% for distances greater than 1 kb. Such wide spacing of library elements is common when the elements are separated by regulatory sequences or when an element is used as a 3′ barcode in an expressed transcript. This causes a serious limit to the size of the viral payload that can be integrated without the unintended consequence of recombination.

Efforts have been made to mitigate this undesired problem. For example, Feldman et al., proposed limiting integrations of multiple lentivirus payloads in a single cell by diluting integrating lentiviral plasmids with an excess of non-integrating lentiviral plasmid DNA. However, this resulted in a 100-fold decrease in viral titer. Even a one-hundred-fold excess of non-integrating DNA still gave an incident of 25% recombination. Accordingly, this is not applicable for libraries beyond a certain level of complexity.

### SUMMARY OF EMBODIMENTS

The disclosure relates to a nucleic acid molecule or a nucleic acid sequence comprising novel virus packaging elements and cells transduced by viruses comprising such elements to enable large-scale library screens with combinatorial elements that heretofore were impractical due to the occurrence of recombination events between species. Recombination events typically occur between library species (or elements) that occur during conventional lentivirus production and transduction of target cells. This is useful in high-throughput screening platforms involving functional genomics. The disclosure relates to new methods of generating highly complex virus libraries with each cell that manufactures a library element to producing only a single type of viral particle, i.e., each cell being homozygous for its viral package and creating a viral particle with two identical or substantially identical packaged nucleic acid sequences.

The disclosure relates to a lentivirus producing cell or cells comprising a viral genome having a single integration DNA element (also referred in this disclosure interchangeably as a “transduction targeting element”). In some embodiments, the transduction targeting element comprises: (a) a first promoter operably linked to a lentiviral long terminal repeat (LTR) and an attB1 element, (b) a second constitutive promoter operably linked to a nucleic acid sequence encoding a detectable marker, or a functional fragment thereof, a nucleic acid sequence encoding a serine recombinase, or a functional fragment thereof, a gene encoding a selectable marker, or a functional fragment thereof, and an inducible promoter operatively linked to a cell death gene or a functional fragment thereof, and (c) an attB2 element, a posttranscriptional regulatory element (PRE) and a 3′ lentiviral LTR. In some embodiments, the cells also comprises a lentivirus helper plasmid that comprises a nucleic acid sequence encoding necessary for the lentivirus life cycle and virion particle formation.

The disclosure also provides a method for producing a library of clonal cells that are each homogeneous for a single integrated lentivirus payload. In some embodiments, the method of the disclosure comprises the steps of: (a) culturing one or a plurality of cells according to the first aspect of the disclosure, (b) transfecting the cells with one or a plurality of plasmids comprising, from 5′ to 3′, a first recombinase attachment element, a payload element and a second recombinase attachment element, wherein the first and second recombinase attachment elements are recognized by the serine recombinase and the payload element comprises a target DNA and a nucleic acid sequence encoding a targeting protein or a functional fragment thereof, (c) allowing the plasmid and the landing pad or transduction targeting element to undergo site-specific recombination in respect to the payload element, (d) inducing expression of the cell death protein to select for cells in which the plasmid and the landing pad have undergone site-specific recombination; and (e) culturing the surviving cells to produce lentivirus particles expressing the payload element.

The disclosure also relates to a nucleic acid sequence comprising: (a) a first expressible nucleic acid and a second expressible nucleic acid; (b) a first regulatory sequence operably linked to the first expressible nucleic acid; (c) a second regulatory sequence operably linked to the second expressible nucleic acid; and (d) a serine recombinase element encoding a serine recombinase positioned on either the first or the second expressible nucleic acid; wherein (a), (b), (c) and (d) are positioned between a viral tandem repeat sequence, such as a 5′ lentiviral LTR and a 3′ lentiviral LTR. In some embodiments, the serine recombinase is a large serine recombinase; wherein the first expressible nucleic acid comprises a first recombinase attachment element; and wherein the second expressible nucleic acid comprises a second recombinase attachment element. In some embodiments, the nucleic acid sequence is positioned within a viral vector, the type of which corresponds to the type of virus from which the viral tandem repeat sequence is derived.

In some embodiments, the serine recombinase element encodes a long serine recombinase or functional variant thereof. In some embodiments, the serine recombinase element comprises SEQ ID NO: 13 or a functional variant thereof comprising at least about 75% sequence identity to SEQ ID NO:13. In some embodiments, the first recombinase attachment element comprises SEQ ID NO: 5 or a functional variant thereof comprising at least about 75% sequence identity to SEQ ID NO: 5; and wherein the second recombinase element comprises SEQ ID NO:6 or a functional variant thereof comprising at least about 75% sequence identity to SEQ ID NO:6.

The disclosure also relates to a nucleic acid sequence comprising: (a) a first expressible nucleic acid and a second expressible nucleic acid; (b) a first regulatory sequence operably linked to the first expressible nucleic acid; (c) a second regulatory sequence operably linked to the second expressible nucleic acid; and (d) a serine recombinase element encoding a serine recombinase positioned on either the first or the second expressible nucleic acid; and (e) a first antibiotic selection nucleic acid sequence positioned on the first or second expressible nucleic acid sequence, wherein (a), (b), (c) and (d) are positioned between a 5′ lentiviral LTR and a 3′ lentiviral LTR. In some embodiments, the antibiotic resistance nucleic acid sequence is BSD or a functional variant thereof. In some embodiments, BSD comprises SEQ ID NO:15 or a functional variant thereof. In some embodiments, the nucleic acid sequence further comprising a negative selection element. In some embodiments, the negative selection element is an inducible caspase selection element positioned on either the first or second expressible nucleic acid sequence. In some embodiments, the inducible caspase election element comprises SEQ ID NO:16 or a functional variant thereof that comprises at least about 75% sequence identity to SEQ ID NO: 16.

The disclosure also relates to any of the above-identified embodiments, wherein the 5′ LTR comprises SEQ ID NO: 17 or a functional variant comprising about 75% sequence identity to SEQ ID NO:17; and wherein the 3′ LTR comprises SEQ ID NO:18 or a functional variant comprising about 75% sequence identity to SEQ ID NO:18. In some embodiments, the nucleic acid sequence further comprises a post-transcriptional regulatory element positioned on the first or second expressible nucleic acid sequences. In some embodiments, the post-transcriptional regulatory element is a WPRE. And in some embodiments, the WPRE comprises SEQ ID NO:9 or a functional variant thereof that comprises at least about 75% sequence identity to SEQ ID NO: 19. In some embodiments, the nucleic acid further comprises a polyadenylation sequence.

The disclosure also relates to a nucleic acid sequence that comprises a first expressible nucleic acid sequence, wherein the first expressible nucleic acid sequence and/or the second expressible nucleic acid sequence comprise a cellular tag. In some embodiments, the cellular tag comprises one or a combination of: a DNA barcode, a nucleic acid encoding a fluorescent protein, or nucleic acid encoding an antigenic tag, In some embodiments, the fluorescent protein comprises: SEQ ID NO: 11 or a functional variant of SEQ ID NO: 11 that comprises at least about 75% sequence identity to SEQ ID NO:11; or comprises SEQ ID NO: 12 or a functional variant of SEQ ID NO:12 that comprise at least about 75% sequence identity to SEQ ID NO: 12. In some embodiments, the first regulatory sequence from cytomegalovirus (CMV).

In some embodiments, the nucleic acid sequence comprises the first regulatory sequence and/or the second regulatory sequence, each comprising one or a combination of nucleic acid sequence chosen from: (x) SEQ ID NO:2 or a functional variant of SEQ ID NO:2 that comprises at least about 75% sequence identity to SEQ ID NO:2; (y) SEQ ID NO:3 or a functional variant of SEQ ID NO:3 that comprises at least about 75% sequence identity to SEQ ID NO:3; and (z) SEQ ID NO: 4 or a functional variant of SEQ ID NO:4 that comprises at least about 75% sequence identity to SEQ ID NO:4.

The disclosure relates to a nucleic acid molecule comprising any of the disclosed nucleic acid sequences. In some embodiments, the nucleic acid molecule is a DNA plasmid, viral vector in single or double stranded form, a cosmid or another molecule. In some embodiments, the nucleic acid molecule further comprises a second antibiotic selection sequence or a first nucleic acid sequence encoding a death domain inhibitor.

The disclosure relates to a cell or cell line comprising any of the nucleic acid sequences or the nucleic acid molecules disclosed herein. In some embodiments, the cell is a 293T cell or 293T cell line.

In some embodiments, the cell comprises a nucleic acid molecule that further comprises a nucleic acid sequence encoding an AAV or lentiviral structural protein. In some embodiments, the disclosure relates to a attenuated or non-attenuated virus that comprises any of the nucleic acid molecules or nucleic acid sequences disclosed herein. In some embodiments, the cell or cell line comprises the nucleic acid molecules or nucleic acid sequences disclosed herein wherein the cell further comprises a nucleic acid molecule encoding structural viral genes that package the nucleic acid molecule or nucleic acid sequence after assembly and secretion of the resulting viral vector. IN some embodiments, the viral vector is a lentiviral vector or an AAV viral vector comprising the nucleic acid molecules disclosed herein. In some embodiments, the cell comprises a nucleic acid sequence identified above or disclosed herein is stably integrated within the endogenous DNA of the cell.

The disclosure relates to a composition comprising any cell or plurality of cells disclosed herein, wherein, if the composition comprises a plurality of cells, the cells are a clonal population, wherein the cell and the cells in the clonal population of cells comprise an identical or substantially identical transduction targeting element, or landing pad. In some embodiments, the cell or cells comprise a transduction targeting element comprises a first promoter, a second promoter, a first recombinase attachment element, a second recombinase attachment element, a first and/or second selection element, and a nucleic acid sequence encoding a recombinase, wherein the nucleic acid encoding the recombinase is positioned between the first recombinase attachment element and the second recombinase attachment element. In some embodiments, the transduction targeting element further comprises a polyadenylation sequence. In some embodiments, the transduction targeting element comprises a polyadenylation sequence positioned 3′ downstream of each other component of the transduction targeting element. In some embodiments, the cell or cells comprise a transduction targeting element comprises a first promoter, a second promoter, a first recombinase attachment element, a second recombinase attachment element, a first and/or second selection element, a nucleic acid sequence encoding a recombinase, and a first and second viral tandem repeat sequence, wherein the nucleic acid encoding the recombinase is positioned between the first recombinase attachment element and the second recombinase attachment element.

In some embodiments, the cell or cells comprise a transduction targeting element comprises a first promoter, a second promoter, a first recombinase attachment element, a second recombinase attachment element, a first and/or second selection element, a nucleic acid sequence encoding a recombinase, and a first and second viral tandem repeat sequences, wherein one of the first or second a promoter, the nucleic acid encoding the recombinase and one of a first or second selection element is positioned between the first recombinase attachment element and the second recombinase attachment element. In some embodiments, the cell or cells comprise a transduction targeting element comprises a first promoter, a second promoter, a first recombinase attachment element, a second recombinase attachment element, a first and/or second selection element, a nucleic acid sequence encoding a recombinase, and a first and second viral tandem repeat sequences, wherein, in 5′ to 3′ orientation, the transduction targeting element components are positioned in an order of: the first promoter, the first viral tandem repeat, the first recombinase attachment element, the second promoter, the nucleic acid sequence encoding the recombinase, the first selection element, the second recombinase attachment element, and the second viral tandem repeat; and wherein, optionally, the transduction targeting element further comprises a WPRE between the second recombinase attachment element and the second viral tandem repeat, and a polyadenylation sequence positioned 3′ downstream from the second viral tandem repeat.

The disclosure relates to a kit comprising: (a) the cell or cell line disclosed herein; and (b) instructions for growing the cell or cell of (a). In some embodiments, the kit further comprising a first container, the container comprising a nucleic acid molecule comprising a payload positioned between a first and second recombinase attachment element. In some embodiments, the kit further comprises a second container comprising a nucleic acid molecule comprising one or a plurality of viral proteins that associate with the 5′ and 3 LTR. In some embodiments, the composition or kit further comprises a cell culture media.

The disclosure relates to a library of viral particles, wherein the viral particles comprise one or a plurality of nucleic acid sequences disclosed herein and/or a payload or protein of interest. In some embodiments, the nucleic acid sequences or molecules provided in the above-identified it are incorporated into a viral particle and supplied as a viral vector in a kit.

The disclosure relates to a method of culturing a cell or cell line comprising: exposing a cell to a cell culture medium under condition sufficient to grow the cell or cell line. In some embodiments, the cell is exposed to 95% oxygen at 37 degrees Celsius for no less than from about 2 to about 10 days. In some embodiments, the method further comprises: exposing the cell or cell line to one or a plurality of nucleic acid molecules comprising a payload positioned between a third and a fourth recombinase attachment element; and (c) allowing a time period sufficient to enable recombination between the nucleic acid molecule comprising the payload and the nucleic acid sequence disclosed herein, such that the payload is exchanged for a region of the transduction targeting element between the first recombinase attachment element and the second recombinase attachment element. In some embodiments, the method further comprises a step (d) transfecting the cell or cell line with a nucleic acid molecule comprising a nucleic acid sequence encoding a viral packing protein after step (a), (b) and (c). In some embodiments, the method further comprises a step (e) culturing the cell or cell line for a time period sufficient for the cell or cell line to produce a virus comprising the viral packaging protein encapsulating two nucleic acid sequence comprising the payload.

The disclosure also relates to a method of preventing homologous recombination in a cell infected with a retrovirus comprising: (a) exposing a cell disclosed herein to a cell culture medium under condition sufficient to grow the cell or cell line. In some embodiments, the method further comprises a step of: exposing the cell or cell line to one or a plurality of nucleic acid molecules comprising a payload positioned between a third and a fourth recombinase attachment element. In some embodiments, the method further comprises a step of: (c) allowing a time period sufficient to enable recombination between the nucleic acid molecule comprising the payload and the nucleic acid sequence disclosed herein, such that the payload is exchanged for a region of the transduction targeting element between the first recombinase attachment element and the second recombinase attachment element. In some embodiments, the method further comprises a step (d) transfecting the cell or cell line with a nucleic acid molecule comprising a nucleic acid sequence encoding viral packaging proteins after step (a), (b) and (c), such that virions are produced by the cell that carry the nucleic acid sequence within the transduction targeting element between the first and second viral tandem repeats and including the payload. In some embodiments, the payload is an antigenic protein, a sequence encoding a chimeric immune receptor, a therapeutic protein, a DNA barcode or a DNA barcode in frame with any one of the foregoing.

The disclosure also relates to a method of modifying the genetic material of a cell comprising: exposing any cell or plurality of cells disclosed herein to a nucleic acid molecule comprising a payload flanked by two recombinase attachment elements capable of aligning to or non covalently binding to a first and a second recombinase attachment element in the genomic DNA of the cell or plurality of cells; allowing the payload to exchange its position from the nucleic acid molecule into the genomic DNA of the cell flanked by the first and second recombinase attachment elements, such that the payload becomes integrated into the genomic DNA of the cell or plurality of cells and the genetic material of the cell becomes modified; wherein, if the composition comprises a plurality of cells, the cells are a clonal population, wherein the cell and the cells in the clonal population of cells comprise an identical or substantially identical transduction targeting element, or landing pad. In some embodiments, the genomic DNA flanked by the first and second recombinase attachment element comprises the nucleic acid sequence encoding the recombinase.

The disclosure also relates to a method of generating a composition comprising a homogenous population of viruses carrying a payload, wherein the viruses comprise an identical or substantially identical viral genome, the method comprising (a) exposing a cell disclosed herein to a cell culture medium under condition sufficient to grow the cell or cell line. In some embodiments, the method further comprises a step of: (a) exposing a cell or cell line disclosed herein to one or a plurality of nucleic acid molecules comprising a payload positioned between a third and a fourth recombinase attachment element. In some embodiments, the method further comprises a step of: (c) allowing a time period sufficient to enable recombination between the nucleic acid molecule comprising the payload and the nucleic acid sequence disclosed herein, such that the payload is exchanged for a region of the transduction targeting element between the first recombinase attachment element and the second recombinase attachment element. In some embodiments, the method further comprises a step (d) transfecting the cell or cell line with a nucleic acid molecule comprising a nucleic acid sequence encoding viral packaging proteins after step (a), (b) and (c), such that virions are produced by the cell that carry the nucleic acid sequence within the transduction targeting element between the first and second viral tandem repeats and including the payload. In some embodiments, the payload is an antigenic protein, a sequence encoding a chimeric immune receptor, a therapeutic protein, a DNA barcode or a DNA barcode in frame with any one of the foregoing.

### BRIEF DESCRIPTION OF THE DRAWINGS

FIGS. 1A and 1B show a schematic of a lentivirus particle. Note the presence of two RNA genomes, which if non-identical can undergo homologous recombination during replication and integration, leading to a hybrid virus with parts that have been exchanged between the two genomes.

FIG. 2 shows how pooled production of large pools of viruses causes cells to take up multiple different copies of plasmid DNA during transfection, resulting in viral particles that carry multiple different elements of the library.

FIGS. 3A and 3B show how generation of clonal virus packaging cells prevents recombination.

FIG. 4 shows one example of a landing pad integrated in the AAVS1 locus of the chromosome.

FIGS. 5A and 5B show how site-specific recombination between the attB and attP sites results in integration of the viral payload into the chromosome and removes the cell death gene from the chromosome.

FIGS. 6A and 6B respectively depict substrates used in and results obtained from flow cytometry-based recombination analysis.

FIGS. 7A and 7B respectively show substrates used in and results obtained from DNA sequencing-based recombination analysis.

FIGS. 8A through 8C. Overall schematic of PRECISE landing pad implementation. (A) PRECISE at genomic safe harbor locus. Expression of LSR and NSG is driven by promoter within the landing pad. (B) Example of an implementation of PRECISE. CMV promoter is used to drive PRECISE locus. StayGold green fluorescent protein is used for labeling PRECISE cells; Pa01 is used as the LSR; blasticidin resistance gene (BSD) is used for selection; inducible caspase-9 (iCasp9) is used as the NSG. (C) Schematic of transfer vector for delivering payload into the PRECISE locus.

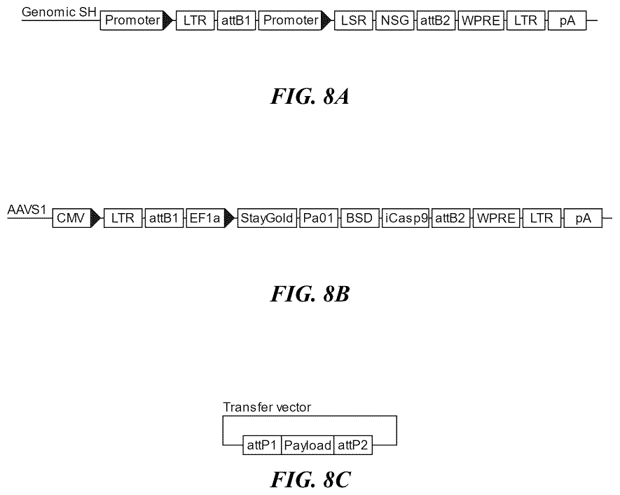

FIGS. 9A and 9B. Schematic for the engineering PRECISE landing pads at AAVS1 genomic safe harbor locus. (A) The PRECISE landing pad construct is carried within an HDR transfer vector and is flanked by two homology arms for the AAVS1 locus (located at chr19; PPP1R12C intron 1). CRISPR/Cas9 is used for directed generation of DNA double strand break at the AAVS1 locus and endogenous HDR machinery within the cell repairs the break using the HDR transfer vector as template, resulting in the integration of the landing pad at the AAVS1 locus. (B) Schematic of the PRECISE landing pad after integration into the AAVS1 locus.

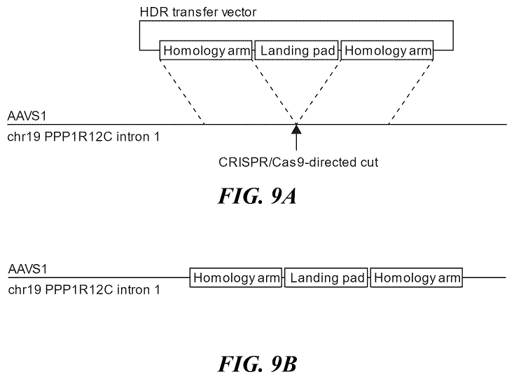

FIGS. 10A and 10B. Schematic for the integration of genetic payload into the PRECISE landing pad. (A) Genetic payload is carried within a transfer vector and is flanked by attP1 and attP2 sequences. Delivery of the transfer vector into cells carrying the PRECISE landing pad results in cassette exchange of the region flanked by attB1 and attB2 within the landing pad with the genetic payload. This results in the integration of the payload into the landing pad and the excision of the promoter-LSR-NSG cassette originally present in the landing pad. The excised sequence is integrated into the transfer vector as extrachromosomal DNA and is lost eventually. (B) Schematic of the viral mRNA transcribed by the PRECISE landing pad after integration of payload.

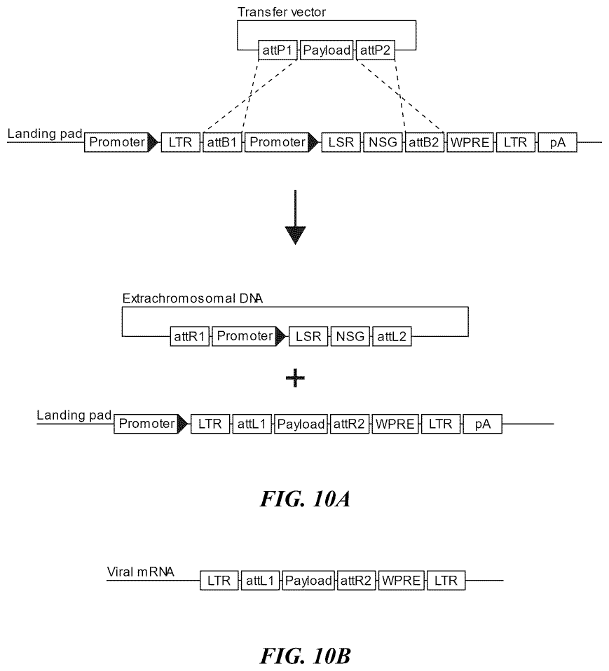

FIGS. 11A and 11B. Schematic of workflow for PRECISE virus production. (A) Cells carrying PRECISE landing pads are transfected with library of transfer vectors and selected for cells that receive successful integration of payload. Transfection of viral packaging plasmids is performed for virus production and virus is harvested from the culture medium. (B) PRECISE cells carry only a single copy of the landing pad and this results in single-copy integration of the payload even if multiple copies of the transfer vector are present within the cell.

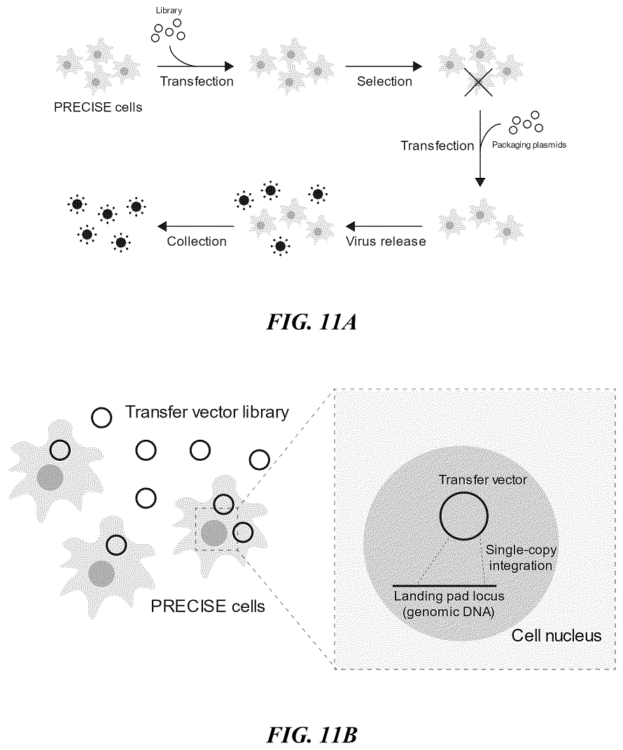

FIGS. 12A through 12C. Example of recombination and barcode uncoupling in conventionally produced retrovirus. (A) Library with payloads that are barcoded with specific barcodes (e.g. 1, 2, 3, etc.). (B) When virus production is performed in a pooled setting, the resulting viral particles end up carrying viral mRNAs that encode different elements in the library. (C) During infection, reverse transcription template switching occurs occasionally, resulting in the uncoupling of the original payload-barcode pairing and the generation of a new payload-barcode pair. This consequently leads to the integration of a new payload that is not originally in the library.

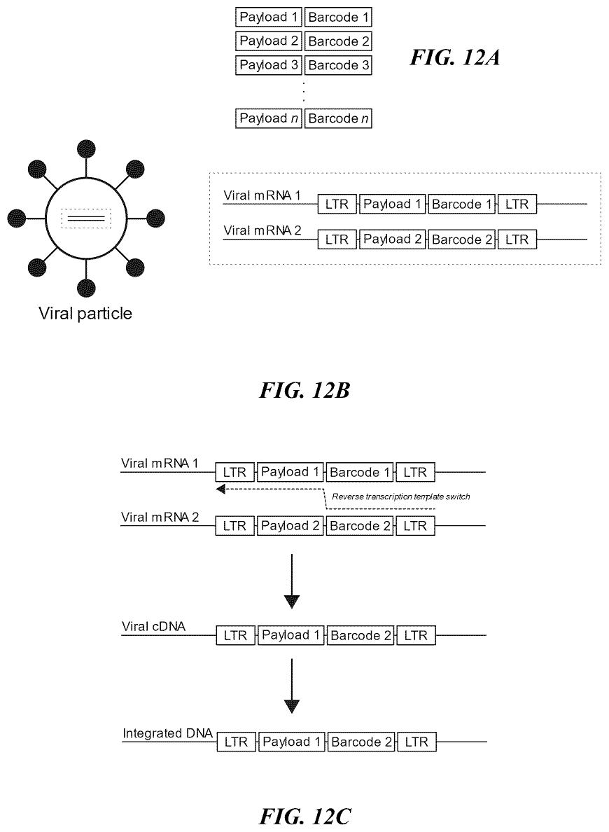

FIGS. 13A and 13B. Example of recombination-free virus generated using PRECISE. (A) Viral particles produced using PRECISE carry two identical viral mRNA due to the clonal nature of the producing cells. (B) During infection, even if reverse transcription template switching occurs, the payload-barcode pairing is maintained owing to both mRNA strands being identical.

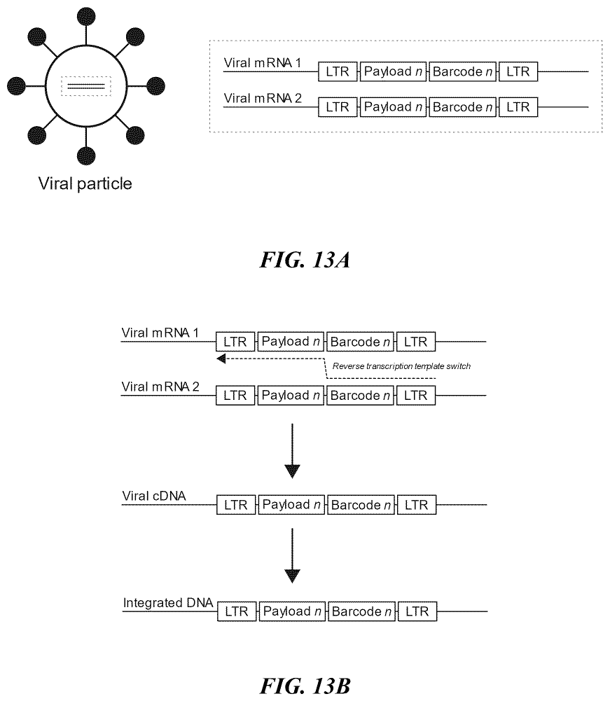

FIGS. 14A through 14D. Example of targeted clonal virus generated using PRECISE. PRECISE can be used to generate viral particles with three unique components: [1] barcode, [2] genetic payload, and [3] targeting protein expressed on the surface of the viral particles. (A) Example of a library with various barcode-payload-targeting elements. (B) When virus is produced in a pool using conventional methods, the resulting viral particles will carry every targeting protein present in the library and mismatched barcodes and payloads. (C) Virus produced using PRECISE will be clonal and will carry only a single targeting protein, along with its associated barcode and genetic payload. (D) Schematic of the payload design in this example.

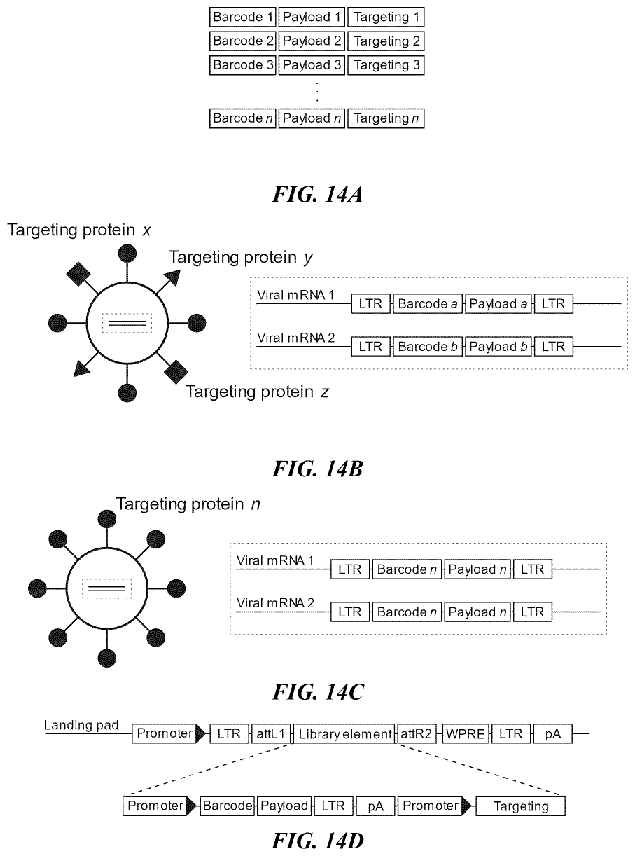

FIGS. 15A and 15 B depict (15A) Generic schematic representing implementation of an adeno-associated virus (AAV) landing pad. Expression of LSR and NSG is driven by promoter within the landing pad. The landing pad cassette is flanked by AAV2 inverted terminal repeats (ITR). (15B) Specific implementation of the AAV landing pad, with CAG driving the expression of StayGold, Pa01, BSD, iCasp9.

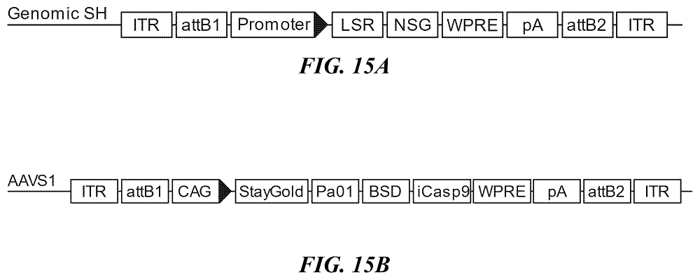

FIGS. 16A and 16B depict (A) Example of the use of recombination-free lentivirus. Here, the identity of each CRISPR sgRNA in a library is coupled with a predetermined protein barcode. Decoupling of sgRNA-barcode pairing is avoided when each viral particle carries identical viral mRNAs that have the same sgRNA-barcode pairing. (B) The same example but with the use of an RNA barcode instead of a protein barcode.

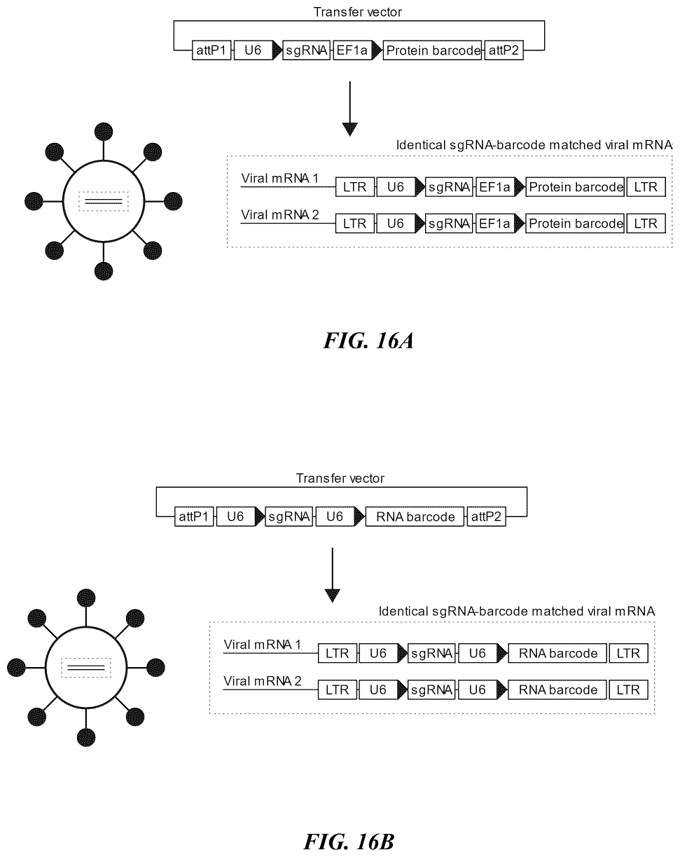

FIGS. 17A through 17C show (FIG. 17A) a generic schematic representing implementation of a general purpose landing pad for viral-like particle production. Expression of LSR and NSG is driven by promoter within the landing pad. The landing pad cassette is flanked by insulators to prevent transcriptional silencing. (FIG. 17B) Specific implementation of the general purpose landing pad, with CAG driving the expression of Pa01, iCasp9, StayGold, and BSD. (FIG. 17C) Transfer vector carrying the protein display cassette to be integrated into the landing pad cells.

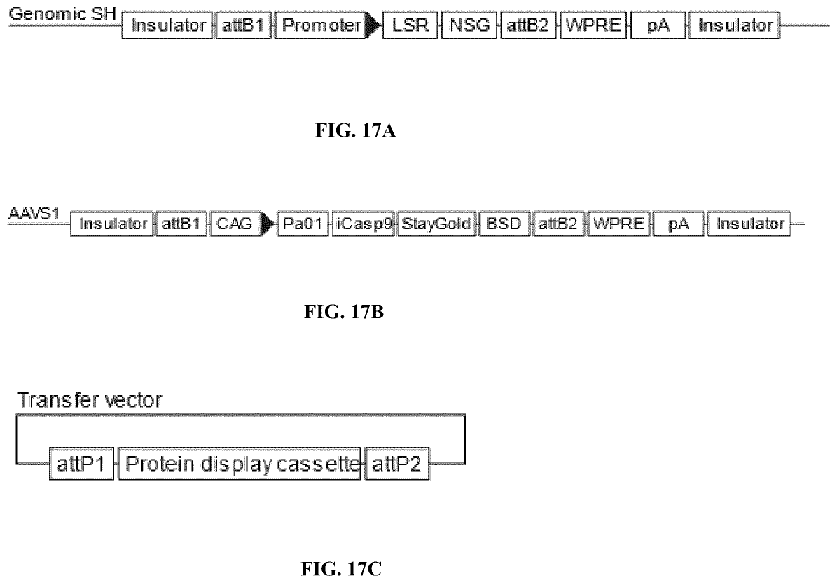

FIGS. 18A and 18B show (FIG. 18A) Clonal production of viral-like particles (VLPs) for surface protein display. Each producer cell carries a single variant of the surface display protein and can only produce a single clonal variant of the viral-like particle carrying the same surface protein. (FIG. 18B) Workflow for clonal production of VLPs. Landing pad cells are first transfected with a plasmid pool comprising a library of surface display proteins of interest. Selection is performed on the cells following integration and VLP production is carried out in a single pool. The VLPs are subsequently collected from the culture supernatant for downstream processing.

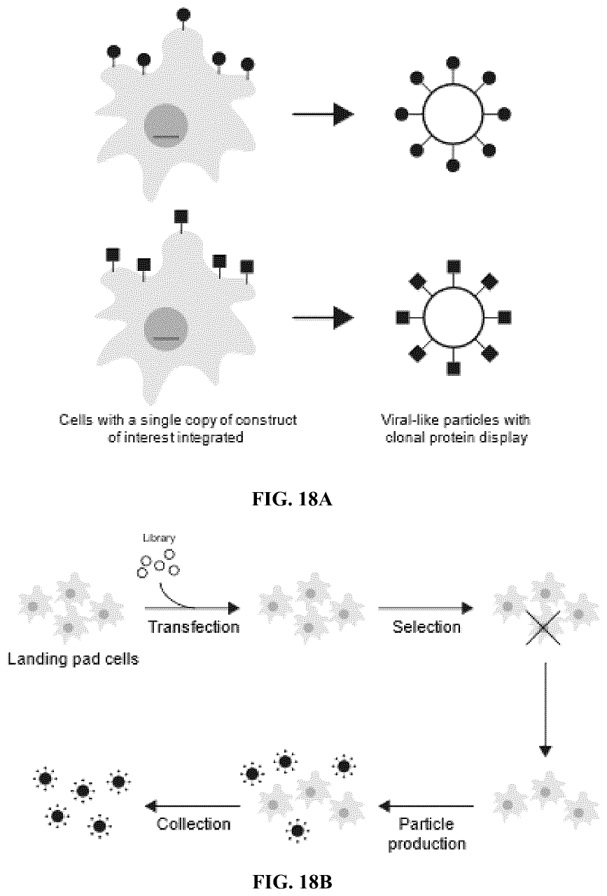

FIGS. 19A through 19C show (FIG. 19A) A generic construct for the production of VLPs with either a viral capsid or engineered nanocages. Protein of interest for surface display is co-expressed with a capsid protein fused to an RNA binding domain (RBD) and RNA barcodes for packaging and export in the VLPs. (FIG. 19B) Illustration of a VLP with surface display protein and RNA barcode providing a phenotype-genotype link. (FIG. 19C) A variant of the VLP with a multipass transmembrane protein instead.

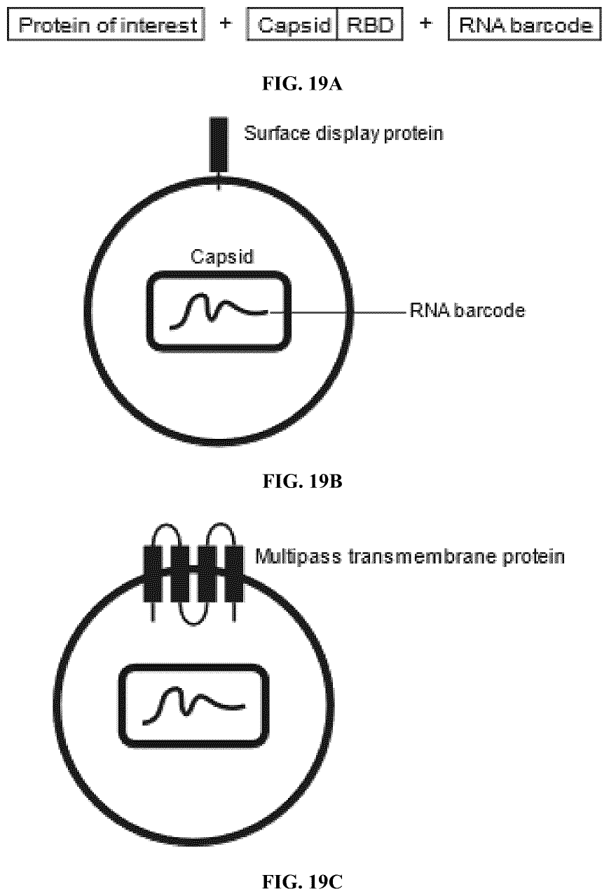

FIGS. 20A through 20C show (FIG. 20A) A generic construct for the capsid-free production of VLPs with the protein of interest linked to an export signal and RNA binding domain (RBD) for RNA barcode export. (FIG. 20B) Illustration of a VLP with surface display protein and RNA barcode providing a phenotype-genotype link. (FIG. 20C) A variant of the VLP with a multipass transmembrane protein instead.

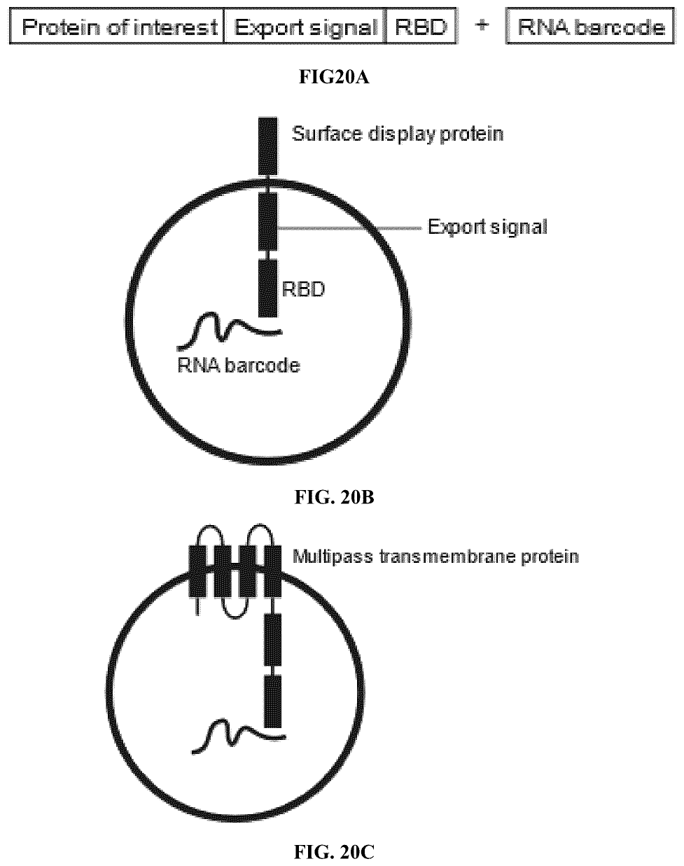

### DETAILED DESCRIPTION

The terminology used herein is for the purpose of describing particular embodiments only and is not intended to be limiting. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the disclosure described herein are capable of operation in other sequences than described or illustrated herein.

The following terms or definitions are provided solely to aid in the understanding of the disclosure. Unless specifically defined herein, all terms used herein have the same meaning as they would to one skilled in the art of the present disclosure. Practitioners are particularly directed to Sambrook et al., Molecular Cloning: A Laboratory Manual, 2nd ed., Cold Spring Harbor Press, Plainsview, N.Y. (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

As used in the specification and the appended claims, the singular forms “a,” “an” and “the” include plural referents unless the context clearly dictates otherwise.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in some embodiments, to A without B (optionally including elements other than B); in another embodiments, to B without A (optionally including elements other than A); in yet another embodiments, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of.” “Consisting essentially of,” when used in the claims, shall have its ordinary meaning as used in the field of patent law.

As used herein, the term “payload element” refers to any nucleic acid sequence that encodes a protein of interest. In some embodiments, the term “protein of interest” refers to any protein that can be expressed in any of the constructs disclosed herein. In some embodiments, the “protein of interest” is a viral antigen. In some embodiments, the “protein of interest” is any of the viral antigens disclosed herein. In some embodiments, the “protein of interest” is a cancer antigen. In some embodiments, the “protein of interest” is a protein associated with the presence of a disease or disorder. In some embodiments, the “protein of interest” is any of the protein that is configured to bind to a probe disclosed herein.

The terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. Thus, the term “expressible nucleic acid” or “expressible nucleic acid sequence” as used herein refers to expressible DNA or RNA molecules or expressible DNA or RNA sequences.

The nucleic acid molecule and/or sequences of each embodiment can be single-stranded or double-stranded. In some embodiments, the nucleic acid molecules of the disclosure comprise a contiguous open reading frame encoding an antibody, or a fragment thereof, as described herein. “Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. A nucleic acid will generally contain phosphodiester bonds, although nucleic acid analogs maybe included that may have at least one different linkage, e.g., phosphoramidate, phosphorothioate, phosphorodithioate, or o-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference in their entireties. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino) propyl uridine, 5-bromo uridine; adenosines and guanosines modified at the 8-position, e.g. 8-bromo guanosine; deaza nucleotides, e.g. 7-deaza-adenosine; 0- and N-alkylated nucleotides, e.g. N6-methyl adenosine are suitable. The 2′-OH-group may be replaced by a group selected from H, OR, R, halo, SH, SR, NH2, NHR, N2 or CN, wherein R is C1-C6 alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature (Oct. 30, 2005), Soutschek et al., Nature 432:173-178 (2004), and U.S. Patent Publication No. 20050107325, which are incorporated herein by reference in their entireties. Modified nucleotides and nucleic acids may also include locked nucleic acids (LNA), as described in US20020115080, which is incorporated herein by reference.

Additional modified nucleotides and nucleic acids are described in U.S. Patent Publication No. 20050182005, which is incorporated herein by reference in its entirety. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made. In some embodiments, the expressible nucleic acid sequence is in the form of DNA. In some embodiments, the expressible nucleic acid is in the form of RNA with a sequence that encodes the polypeptide sequences disclosed herein and, in some embodiments, the expressible nucleic acid sequence is an RNA/DNA hybrid molecule that encodes any one or plurality of polypeptide sequences disclosed herein.

As used herein, the term “nucleic acid molecule” is a molecule that comprises one or more nucleotide sequences that encode one or more proteins. In some embodiments, a nucleic acid molecule comprises initiation and termination signals operably linked to regulatory elements including a promoter and polyadenylation signal capable of directing expression in the cells of the individual to whom the nucleic acid molecule is administered. In some embodiments, the nucleic acid molecule also includes a plasmid containing one or more nucleotide sequences that encode one or a plurality of structural and packaging proteins needed for expression and secretion of virion protein components. In some embodiments, the disclosure relates to a composition comprising a cell, the cell comprising a first, second, third or more nucleic acid molecule, each of which encoding one or a plurality of: a Cas protein, a sgRNA, and a nucleic acid molecule comprising a transduction targeting element disclosed herein, and at least one of each plasmid comprising one or more of the compositions disclosed herein. In some embodiments, the compositions can comprise a nucleic acid molecule that comprises a first, second, third or more expressible nucleic acid sequences, wherein at least one of the first, second or third expressible nucleic acid sequences comprise the domains disclosed herein. In some embodiments the nucleic acid molecule comprises a transduction targeting element sequence configured with a Cas protein recognition sequence capable of recombination even with the genomic DNA of the cell. The resulting reaction with a CRISPR system and a disclosed cell is that genomic DNA of the cell is modified to include a transduction targeting element.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present disclosure is not entitled to antedate such disclosure by virtue of prior disclosure. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

“AAV virion” refers to a complete virus particle, such as for example a wild type AAV virion particle, which comprises single stranded genome DNA packaged into AAV capsid proteins. The single stranded nucleic acid molecule is either sense strand or antisense strand, as both strands are equally infectious. In some embodiments, viral vectors are AAV virions comprising identical or substantially identical strands. In some embodiments, the viral vectors are identical and derived from or manufactured from a cell expressing identical or substantially identical nucleic acid sequence strands comprising a nucleic acid sequence encoding the target protein. “rAAV virion” refers to a recombinant AAV virus particle, i.e. a particle which is infectious but replication defective. It is composed of an AAV protein shell and comprises a rAAV vector. In the context of the present disclosure the protein shell may be of a different serotype than the rAAV vector. An AAV virion of the disclosure may thus be composed a protein shell, i.e. the icosahedral capsid, which comprises capsid proteins (VP1, VP2, and/or VP3) of one AAV serotype, e.g. AAV serotype 6, whereas the rAAV vector contained in that AAV6 virion may be any of the rAAVX vectors described above, including a rAAV6 vector. An “rAAV6 virion” comprises capsid proteins of AAV serotype 6, while e.g. a rAAV2 virion comprises capsid proteins of AAV serotype 2, whereby either may comprise any of rAAVX vectors of the disclosure. “AAV helper functions” generally refers to the corresponding AAV functions required for rAAV replication and packaging supplied to the rAAV virion or rAAV vector in trans. AAV helper functions complement the AAV functions which are missing in the rAAV vector, but they lack AAV ITRs (which are provided by the rAAV vector). AAV helper functions include the two major ORFs of AAV, namely the rep coding region and the cap coding region or functional substantially identical sequences thereof. Rep and Cap regions are well known in the art, see e.g. Chiorini et al. (1999, J. of Virology, Vol 73 (2): 1309-1319) or U.S. Pat. No. 5,139,941, incorporated herein by reference. The AAV helper functions can be supplied on a AAV helper construct. Introduction of the helper construct by into the host cell can occur e.g. by transformation or transduction prior to or concurrently with the introduction of the rAAV vector. The AAV helper constructs of the disclosure may thus be chosen such that they produce the desired combination of serotypes for the rAAV virion's capsid proteins on the one hand and for the rAAV vector replication and packaging on the other hand.

“AAV helper virus” provides additional functions required for AAV replication and packaging. Suitable AAV helper viruses include adenoviruses, herpes simplex viruses (such as HSV types 1 and 2) and vaccinia viruses. The additional functions provided by the helper virus can also be introduced into the host cell via vectors, as described in U.S. Pat. No. 6,531,456 incorporated herein by reference.

The term “about” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods. For recitation of numeric ranges herein, each intervening number therebetween with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

“Cell type” means the organism, organ, and/or tissue type from which the cell is derived or sourced, state of development, phenotype or any other categorization of a particular cell that appropriately forms the basis for defining it as “similar to” or “different from” another cell or cells.

“Coding sequence” or “encoding nucleic acid” as used herein may mean refers to the nucleic acid (RNA, DNA, or RNA/DNA hybrid molecule) that comprises a nucleotide sequence which encodes a protein. The coding sequence may further include initiation and termination signals operably linked to regulatory elements including a promoter and polyadenylation signal capable of directing expression in the cells of an individual or mammal to whom the nucleic acid is administered.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

As used herein, the term “functional fragment” means any portion of a polypeptide that is of a sufficient length to retain at least partial biological function that is similar to or substantially similar to the wild-type polypeptide upon which the fragment is based. In some embodiments, a functional fragment of a polypeptide is a polypeptide that comprises or possesses 80, 85, 90, 95, 96, 97, 98, or 99% sequence identity to any polypeptide disclosed in Table Z and has sufficient length to retain at least partial binding affinity to one or a plurality of ligands that bind to the polypeptides in Table Z. In some embodiments, a functional fragment of a nucleic acid is a nucleic acid that comprises or possesses 80, 85, 90, 95, 96, 97, 98, or 99% sequence identity to any nucleic acid to which it is being compared and has sufficient length to retain at least partial function related to the nucleic acid to which it is being compared. In some embodiments, the fragment is a fragment of any polypeptide disclosed in Table Z and has a length of at least about 10, about 20, about 30, about 40, about 50, about 60, about 70, about 80, about 90, or about 100 contiguous amino acids. In some embodiments, the fragment is a fragment of any polypeptide disclosed in Table Z and has a length of at least about 50 amino acids. In some embodiments, the fragment is a fragment of any polypeptide disclosed in Table Z and has a length of at least about 100 amino acids. In some embodiments, the fragment is a fragment of any polypeptide disclosed in Table Z and has a length of at least about 150 amino acids. In some embodiments, the fragment is a fragment of any polypeptide disclosed in Table Z and has a length of at least about 200 amino acids. In some embodiments, the fragment is a fragment of any polypeptide disclosed in Table Z and has a length of at least about 250 amino acids. In some embodiments, the fragment is a fragment of any polypeptide disclosed in Table Z and has a length of at least about 300 amino acids. In some embodiments, the fragment is a fragment of any polypeptide disclosed in Table Z and has a length of at least about 350 amino acids. In some embodiments, the fragment is a fragment of any polypeptide disclosed in Table Z and has a length of at least about 400 amino acids. In some embodiments, the fragment is a fragment of any polypeptide disclosed in Table Z and has a length of at least about 450 amino acids. In some embodiments, the fragment is a fragment of any polypeptide disclosed in Table Z and has a length of at least about 500 amino acids. In some embodiments, the fragment is a fragment of any polypeptide disclosed in Table Z and has a length of at least about 550 amino acids.

As used herein, “expression” refers to the process by which a polynucleotide is transcribed from a DNA template (such as into and mRNA or other RNA transcript) and/or the process by which a transcribed mRNA (or administered mRNA) is translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. In some embodiments, the at least first expressible nucleic acid sequence comprises only DNA nucleotides, RNA nucleotides or comprises both RNA and DNA nucleotides. In some embodiments, the at least first expressible nucleic acid consist of RNA. In some embodiments, the at least first expressible nucleic acid consist of DNA.

A “lipoparticle,” as that term is used herein, means a small particle from about a nanometer to about one micrometer, comprising a lipid bilayer comprising a protein capable of interacting with a cognate ligand essentially as it would otherwise interact with the ligand when the protein is present in an intact membrane. The lipoparticle does not encompass cell membrane vesicles, which are typically produced using empirical methods and which are usually heterogeneous in size. The lipoparticle of the disclosure is, in some embodiments, dense, spherical and/or homogeneous in size.

A “viral particle” means lipoparticle, pseudovirus, or a small particle, from about a nanometer to about one micrometer, comprising a lipid bilayer comprising a viral vector that comprises a an expressible, viral nucleic acid sequence encoding at least one protein capable of interacting with a cognate ligand essentially as it would otherwise interact with the ligand when the protein is present in an intact membrane.

Some embodiments are pseudotyped viral particles if they comprise at least two proteins from two different viruses, such as a lentiviral protein and a flaviviral protein or a combination of two different viral proteins. Although an enveloped virus preferentially incorporates its own viral envelope protein(s) into the envelope during virus assembly, the tropism of a number of enveloped viruses may be altered when a different viral envelope glycoprotein is incorporated into the envelope during virus assembly by a process called phenotypic mixing or pseudotyping. Virus pseudotypes may be formed by co-infection of a cell by two different enveloped viruses or may be generated experimentally by expressing a viral envelope protein encoded by one virus in a cell infected with another virus. Pseudotype formation in vivo has been postulated to enhance or alter the pathologic potential of an enveloped virus. In some embodiments, the viral particle disclosed herein or libraries disclosed herein comprises or are pseudotyped viruses that comprise at least one combination expressible viral polypeptides from two different viruses. In some embodiments, the viral particle is replication deficient.

The term “polypeptide” encompasses two or more naturally or non-naturally-occurring amino acids joined by a covalent bond (e.g., an amide bond). Polypeptides as described herein include full-length proteins (e.g., fully processed pro-proteins or full-length synthetic polypeptides) as well as shorter amino acid sequences (e.g., fragments of naturally-occurring proteins or synthetic polypeptide fragments).

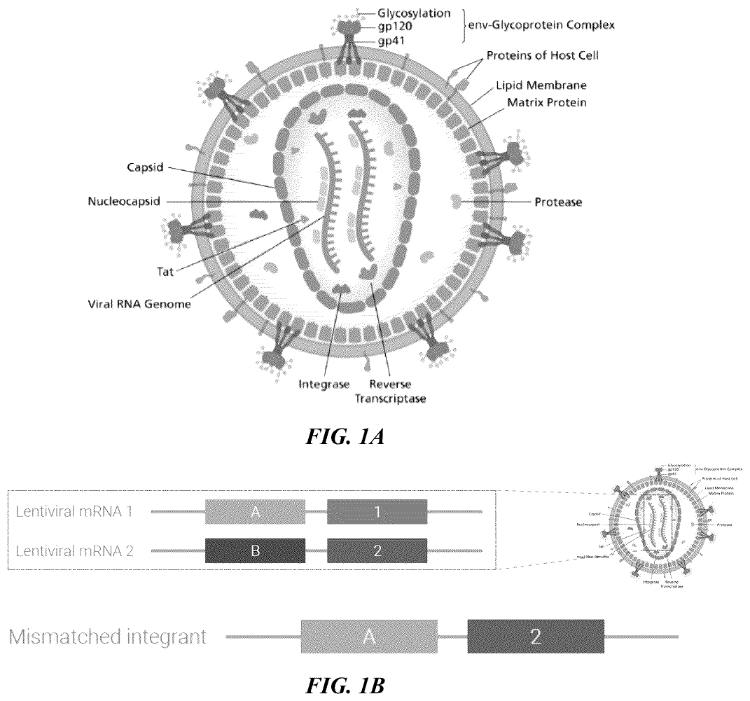

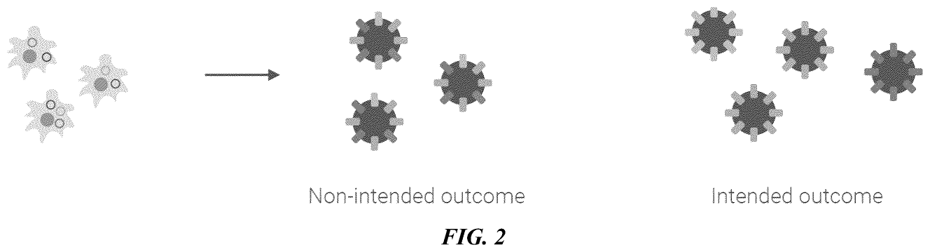

The disclosure relates to a viral vector, such as a retroviral vector or an adenoviral vector or an AAV vector. In some embodiments, the disclosure relates to a composition comprising a nucleic acid sequence encoding a viral packaging protein from Retroviridae, AAV, adenovirus, Flavivirus. The disclosure also relates to a nucleic acid sequence comprising a retroviral LTR or a viral particle. The disclosure relates to a retroviral vector are widely used for functional genomic screens, enabling efficient and stable transduction of target cells with libraries of genetic elements. Unfortunately, designs that rely upon integrating multiple variable sequences, such as combinatorial perturbations or perturbations linked to barcodes may be compromised by unintended consequences of lentiviral packaging. Intermolecular recombination between library elements and integration of multiple perturbations, even at limiting virus dilution, can negatively impact the sensitivity of pooled screens. Recombination can arise from the template-switching of the lentiviral reverse-transcriptase. As the lentivirus capsid normally packages a dimer of RNA genomes, intermolecular recombination can occur in target cells infected by a single virion (FIGS. 1A and 1B). The fraction of target cells with recombined integrants depends on the distance between variable sequences and has been measured to exceed 30% for distances greater than 1 kb. Such wide spacing of library elements is common when the elements are separated by regulatory sequences or when an element is used as a 3′ barcode in an expressed transcript. This causes a serious limit to the size of the viral payload that can be integrated without the unintended consequence of recombination. In addition, current methods do not allow the pooled production of large pools of viruses containing different targeting proteins because the resulting viral particles will carry multiple elements from the library (FIG. 2). There is, therefore, a need for new ways to generate highly complex libraries with each cell producing only one type of viral particle, i.e., each cell being homozygous for its viral package.

Any probes may be used in concert with any of the devices, systems, kits, or methods disclosed herein. As used herein, the term “probe” refers to any molecule that may bind or associate, indirectly or directly, covalently or non-covalently, to any of the substrates and/or reaction products and/or proteases disclosed herein and whose association or binding is detectable using the methods disclosed herein. In some embodiments, the probe is a fluorogenic, fluorescent, or chemiluminescent probe, an antibody, or an absorbance-based probe. In some embodiments, an absorbance-based probe, for example the chromophore pNA (para-nitroanaline), may be used as a probe for detection and/or quantification of a protease disclosed herein. In some embodiments, the probe comprises an amino acid sequence that is a expressed by the cell comprising a transduction targeting element comprise at least about 70%, at least about 75%, at least about 80%, at least about 85%, at least about 87%, at least about 90%, at least about 95%, at least about 96%, at least about 97%, at least about 98%, or at least about 99% sequence identity to amino acids SEQ ID NO: 24 through 34. A probe may also be considered a nucleic acid sequence within the transduction targeting element such as SEQ ID NO:23. A probe may also be a molecule that binds any of the expressed protein of the disclosed cells, such that, when characterizing the cells or viruses disclosed herein, method may comprise a step of detecting or quantifying the presence of the probe bound covalently or non-covalently to the cell or virus. A probe may be immobilized, adsorbed, or otherwise non-covalently bound to a solid surface, such that upon exposure to an enzyme for a time period sufficient to perform an enzymatic reaction, it can be enzymatically cleaved. In some embodiments, cleavage of the substrate causes a biological change in the nature or chemical availability of one or more probes such that cleavage enables detection of the reaction product. For instance, if the step of detecting comprises use of FRET, cleavage of the substrate disclosed herein causes one of the chromophore to emit a fluorescent light under exposure to a wavelength sufficient to activate such a fluorescent molecule. The intensity, length, or amplitude of a wavelength emitted from fluorescent marker can be measured and is, in some embodiments, proportional to the presence, absence or quantity of enzyme present in the reaction vessel, thereby the quantity of enzyme can be determined from detection of the intensity of or fluorescence at a known wavelength of light. A probe may also be bound to a surface about which cells or viruses of the disclosure are exposed, such as flow cytometry, during which cells are characterized based upon their relative levels of probe binding to the cell or viruses. Probes of the disclosure also include dyes and other small molecules who after stimulation with wavelengths of length emit a visible color or measurable wavelength of light under conditions conducive to observation. Probes of the disclosure also include antibodies, including, in some embodiments, antibodies that comprises one or more complementary determinant region (CDR) specific to an expressed payload, an expressed protein on a cell or virus disclosed herein.

“Variant” used herein with respect to a nucleic acid means a nucleic acid sequence comprising (i) a portion or fragment of a referenced nucleotide sequence; (ii) the complement of a referenced nucleotide sequence or portion thereof; (iii) a nucleic acid sequence that is substantially identical to a referenced nucleic acid or the complement thereof; or (iv) a nucleic acid sequence that hybridizes under stringent conditions to the referenced nucleic acid, complement thereof, or a sequences substantially identical thereto. “Variant” with respect to a peptide or polypeptide that differs in amino acid sequence by the insertion, deletion, truncation, conservative substitution of amino acids, or addition of at least one amino acid as compared to a reference sequence, but the peptide or polypeptide retains at least one biological activity of the reference sequence upon which it is based. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. Nucleic acid molecules or nucleic acid sequences of the disclosure include those that encode amino acid sequences comprising one or more of: SEQ ID NO:24, SEQ ID NO:36, SEQ ID NO:42, SEQ ID NO: 46 and variants or functional fragments thereof that possess no less than about 70%, about 75%, about 80%, about 85%, about 90%, about 95%, about 96%, about 97%, about 98%, or about 99% sequence identity with the coding sequences of the foregoing. The term “variant” includes polypeptides conjugated to a non-natural chemical moieties or variants. In some embodiments, the polypeptide comprises a polymer, such as polyethylene glycol, and may be comprised of one or more additional derivitizations of cysteine, lysine, or other residues. In addition, variants of the instant disclosure may comprise a linker or polymer, wherein the amino acid to which the linker or polymer is conjugated may be a non-natural amino acid, or may be conjugated to a naturally encoded amino acid utilizing techniques known in the art such as coupling to lysine or cysteine.

### Compositions

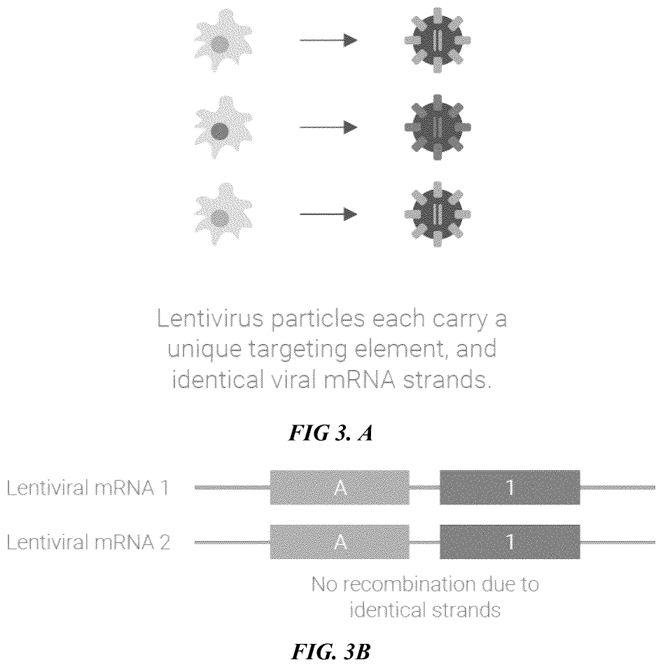

The disclosure provides novel virus packaging elements and cells transduced by such elements to enable large-scale library screens with combinatorial elements that heretofore were impractical due to the presence of recombination between library elements that occurs during conventional lentivirus production and transduction of target cells. The present disclosure overcomes these problems by generating clonal virus packaging cells that each produce a unique virus (FIGS. 3A and 3B). This is particularly useful in high-throughput screening platforms involving functional genomics.

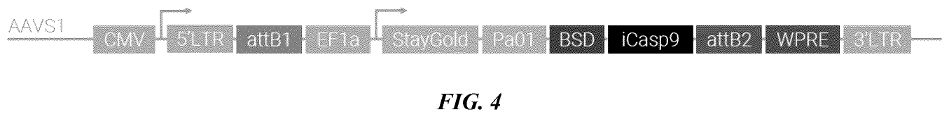

In some aspects, the disclosure provides viral particle-producing cells comprising a genome having a single-integrated DNA element (“landing pad cells”) comprising (a) a first promoter operably linked 5′ to 3′, to a 5′ lentiviral long terminal repeat (LTR) and an attB1 element, (b) a second constitutive promoter operably linked to, in any order, a gene encoding a detectable marker, or a functional fragment thereof, a gene encoding a serine recombinase, or a functional fragment thereof, a gene encoding a selectable marker, or a functional fragment thereof, and an inducible promoter operatively linked to a cell death gene or a functional or a functional thereof, and (c) 5′ to 3′, an attB2 element, a posttranscriptional regulatory element (PRE) and a 3′ lentiviral LTR. One example of such a landing pad is shown in FIG. 4. The landing pad cells also contain lentivirus helper plasmids that contain genes necessary for the lentivirus life cycle and virion particle formation. In some embodiments, the cells express and comprise a nucleic acid sequence encoding clonal viral particles, which comprise lentiviral proteins or AAV particles or combinations thereof. In some embodiments, the lentiviral proteins are HIV1 or HIV2 glycoproteins or ENV proteins or functional variants thereof. In some embodiments, the lentiviral proteins are HIV1 or HIV2 membrane-bound proteins or functional variants thereof expressed on the lipid bilayer of the virus (or of the cell prior budding from the cell).

In some embodiments, substituent (b) further comprises a gene encoding a detectable marker. In some embodiments, the first promoter is selected from a cytomegalovirus (CMV) promoter, human elongation factor-1 alpha (EF1a) promoter, spleen focus-forming virus (SFFV) promoter, cytomegalovirus immediate-early enhancer/chicken β-actin (CAG) promoter, Rous sarcoma virus (RSV) promoter, murine stem cell virus (MSCV) promoter. In some embodiments, the second promoter is a constitutive promoter selected from cytomegalovirus (CMV) promoter, human elongation factor-1 alpha (EF1a) promoter, spleen focus-forming virus (SFFV) promoter, cytomegalovirus immediate-early enhancer/chicken β-actin (CAG) promoter, Rous sarcoma virus (RSV) promoter, murine stem cell virus (MSCV) promoter. In some embodiments, the serine recombinase is selected from Pa01, Bxb1, PhiC31, TP901, Si74, U153. AttB1 and attB2 are the recognition sites for the serine recombinase. In some embodiments, the selectable marker is an antibiotic resistance protein. In some embodiments, the antibiotic resistance protein is selected from a blasticidin S-resistance deaminase (BSD or BSR), puromycin N-acetyl-transferase (pac), ble-Sh, neo-r. As used herein, a “cell death gene” is a gene encoding a protein or functional fragment thereof that, when expressed, causes cell death or stasis. The cell death gene is conditionally activated using an inducible system selected from Tet response element (promoter), E. coli dihydrofolate reductase (ecDHFR; degron), chemical inducer of dimerization (CID; activator), in the presence of its activating ligand, which is selected respectively from tetracycline or doxycycline, trimethoprim, AP1903 or rimiducid. The cell death gene is selected from inducible Caspase 9 (iC9), herpes-simplex-thymidine-kinase (HSV-TK), diphtheria toxin fragment A (DTA). In some embodiments, PRE is selected from Woodchuck Hepatitis virus Posttranscriptional Regulatory Element (WPRE), or functional fragments thereof. When present, the detectable marker is a fluorescence marker or a functional fragment thereof. In some embodiments, the fluorescence marker, when present, is selected from StayGold, mStayGold, mScarlet, mScarlet3, enhanced green fluorescent protein (eGFP), mNeonGreen, mCherry, mCherry2, mTagBFP, mTagBFP2. In some embodiments the landing pad is integrated at the adeno-associated virus integration site 1 (AAVS1) Each list of substituents provided herein is intended to be exemplary and non-limiting.

The disclosure also relates to a nucleic acid sequence that comprises a transduction targeting element and cells comprising the transduction targeting element. Nucleic acid sequences and molecules of the disclosure may comprise any number of elements such as those on Table Z.

TABLE Z
Nucleic Acid and Amino Acid Sequence components.

Lentiviral LTR
gggtctctctggttagaccagatctgagcctgggagctctctggctaactagggaacccactgcttaagcctcaataaagcttgccttgagtgct
tcaag tagtgtgtgcccgtctgttgtgtgactctggtaactagagatccctcagacccttttagtcagtgtggaaaatctctagca (SEQ
ID NO: 1)
Adeno-associated virus ITR--Inverted terminal repeat (ITR)
cctgcaggcagctgcgcgctcgctcgctcactgaggccgcccgggcaaagcccgggcgtcgggcgacctttggtcgcccggcctc
agtgagcgagcgagcgcgcagagagggagtggccaactccatcactaggggttcct (SEQ ID NO: 2)
CMV Promoter
gacattgattattgactagttattaatagtaatcaattacggggtcattagttcatagcccatatatggagttccgcgttacataacttacggta
aatggcccgcctggctgaccgcccaacgacccccgcccattgacgtcaataatgacgtatgttcccatagtaacgccaatagggacttt
ccattgacgtcaatgggtggagtatttacggtaaactgcccacttggcagtacatcaagtgtatcatatgccaagtacgccccctattgac
gtcaatgacggtaaatggcccgcctggcattatgcccagtacatgaccttatgggactttcctacttggcagtacatctacgtattagtcat
cgctattaccatggtgatgcggttttggcagtacatcaatgggcgtggatagcggtttgactcacggggatttccaagtctccaccccatt
gacgtcaatgggagtttgttttggcaccaaaatcaacgggactttccaaaatgtcgtaacaactccgccccattgacgcaaatgggcggt
aggcgtgtacggtgggaggtctatataagcagcgcg (SEQ ID NO: 3)
EF1a Promoter
gggcagagcgcacatcgcccacagtccccgagaagttggggggaggggtcggcaattgaaccggtgcctagagaaggtggcgcg
gggtaaactgggaaagtgatgtcgtgtactggctccgcctttttcccgagggtgggggagaaccgtatataagtgcagtagtcgccgtg
aacgttctttttcgcaacgggtttgccgccagaacacaggtaagtgccgtgtgtggttcccgcgggcctggcctctttacgggttatggc
ccttgcgtgccttgaattacttccacctggctgcagtacgtgattcttgatcccgagcttcgggttggaagtgggtgggagagttcgaggc
cttgcgcttaaggagccccttcgcctcgtgcttgagttgaggcctggcctgggcgctggggccgccgcgtgcgaatctggtggcacct
tcgcgcctgtctcgctgctttcgataagtctctagccatttaaaatttttgatgacctgctgcgacgctttttttctggcaagatagtcttgtaa
atgcgggccaagatctgcacactggtatttcggtttttggggccgcgggcggcgacggggcccgtgcgtcccagcgcacatgttcggc
gaggcggggcctgcgagcgcggccaccgagaatcggacgggggtagtctcaagctggccggcctgctctggtgcctggcctcgc
gccgccgtgtatcgccccgccctgggcggcaaggctggcccggtcggcaccagttgcgtgagcggaaagatggccgcttcccggc
cctgctgcagggagctcaaaatggaggacgcggcgctcgggagagcgggcgggtgagtcacccacacaaaggaaaagggccttt
ccgtcctcagccgtcgcttcatgtgactccacggagtaccgggcgccgtccaggcacctcgattagttctcgcgcttttggagtacgtcg
tctttaggttggggggaggggttttatgcgatggagtttccccacactgagtgggtggagactgaagttaggccagcttggcacttgatg
taattctccttggaatttgccctttttgagtttggatcttggttcattctcaagcctcagacagtggttcaaagtttttttcttccatttcag
(SEQ ID NO: 4)
SSFV promoter
gcagtttcttaagacccatcagatgtttccaggctcccccaaggacctgaaatgaccctgcgccttatttgaattaaccaatcagcctgctt
ctcgcttctgttcgcgcgcttctgcttcccgagctctataaaagagctcacaacccctcactcggcgcgccagtcctccgacagactgag
tcgcccggg (SEQ ID NO: 5)
CAG promoter
cgttacataacttacggtaaatggcccgcctggctgaccgcccaacgacccccgcccattgacgtcaataatgacgtatgttcccatagt
aacgccaatagggactttccattgacgtcaatgggtggagtatttacggtaaactgcccacttggcagtacatcaagtgtatcatatgcca
agtacgccccctattgacgtcaatgacggtaaatggcccgcctggcattatgcccagtacatgaccttatgggactttcctacttggcagt
acatctacgtattagtcatcgctattaccatggtcgaggtgagccccacgttctgcttcactctccccatctcccccccctccccaccccca
attttgtatttatttattttttaattattttgtgcagcgatgggggcggggggggggggggggcgcgcgccaggcggggcggggcggg
gcgaggggcggggcggggcgaggcggagaggtgcggcggcagccaatcagagcggcgcgctccgaaagtttccttttatggcga
ggcggcggcggcggcggccctataaaaagcgaagcgcgcggcggggcgggagtcgctgcgcgctgccttcgccccgtgccccgc
tccgccgccgcctcgcgccgcccgccccggctctgactgaccgcgttactcccacaggtgagcgggcgggacggcccttctcctcc
gggctgtaattagcgcttggtttaatgacggcttgtttcttttctgtggctgcgtgaaagccttgaggggctccgggagggccctttgtgcg
gggggagcggctcggggggtgcgtgcgtgtgtgtgtgcgtggggagcgccgcgtgcggctccgcgctgcccggcggctgtgagc
gctgcgggcgcggcgcggggctttgtgcgctccgcagtgtgcgcgaggggagcgcggccgggggcggtgccccgcggtgcgg
ggggggctgcgaggggaacaaaggctgcgtgcggggtgtgtgcgtgggggggtgagcagggggtgtgggcgcgtcggtcgggc
tgcaaccccccctgcacccccctccccgagttgctgagcacggcccggcttcgggtgcggggctccgtacggggcgtggcgcggg
gctcgccgtgccgggcggggggtggcggcaggtgggggtgccgggcggggcggggccgcctcgggccggggagggctcggg
ggaggggcgcggcggcccccggagcgccggcggctgtcgaggcgcggcgagccgcagccattgccttttatggtaatcgtgcga
gagggcgcagggacttcctttgtcccaaatctgtgcggagccgaaatctgggaggcgccgccgcaccccctctagcgggcgcggg
gcgaagcggtgcggcgccggcaggaaggaaatgggcggggagggccttcgtgcgtcgccgcgccgccgtccccttctccctctcc
agcctcggggctgtccgcggggggacggctgccttcgggggggacggggcagggcggggttcggcttctggcgtgtgaccggcg
gctctagagcctctgctaaccatgttcatgccttcttctttttcctacag (SEQ ID NO: 6)
CbH promoter
cgttacataacttacggtaaatggcccgcctggctgaccgcccaacgacccccgcccattgacgtcaatagtaacgccaatagggact
ttccattgacgtcaatgggtggagtatttacggtaaactgcccacttggcagtacatcaagtgtatcatatgccaagtacgccccctattga
cgtcaatgacggtaaatggcccgcctggcattgtgcccagtacatgaccttatgggactttcctacttggcagtacatctacgtattagtca
tcgctattaccatggtcgaggtgagccccacgttctgcttcactctccccatctcccccccctccccacccccaattttgtatttatttatttt
ttaattattttgtgcagcgatgggggcggggggggggggggggcgcgcgccaggcggggcggggcggggcgaggggcggggcg
gggcgaggcggagaggtgcggcggcagccaatcagagcggcgcgctccgaaagtttccttttatggcgaggcggcggcggcggc
ggccctataaaaagcgaagcgcgcggcgggcgggagtcgctgcgcgctgccttcgccccgtgccccgctccgccgccgcctcgc
gccgcccgccccggctctgactgaccgcgttactcccacaggtgagcgggcgggacggcccttctcctccgggctgtaattagctga
gcaagaggtaagggtttaagggatggttggttggtggggtattaatgtttaattacctggagcacctgcctgaaatcactttttttcaggttg
g (SEQ ID NO: 7)
RSV promoter
tgtagtcttatgcaatactcttgtagtcttgcaacatggtaacgatgagttagcaacatgccttacaaggagagaaaaagcaccgtgcatg
ccgattggtggaagtaaggtggtacgatcgtgccttattaggaaggcaacagacgggtctgacatggattggacgaaccactgaattg
ccgcattgcagagatattgtatttaagtgcctagctcgatacataaac (SEQ ID NO: 8)
MSCV promoter
aatgaaagaccccacctgtaggtttggcaagctagcttaagtaacgccattttgcaaggcatggaaaatacataactgagaatagagaagttca
gatcaaggttaggaacagagagacagcagaatatgggccaaacaggatatctgtggtaagcagttcctgccccggctcagggccaagaacagat
ggtccccagatgcggtcccgccctcagcagtttctagagaaccatcagatgtttccagggtgccccaaggacctgaaatgaccctgtgccttatt
tgaactaaccaatcagtt cgcttctcgcttctgttcgcgcgcttctgctccccgagctcaataaaagagcccacaacccctcactcggcgcgc
cagtc (SEQ ID NO: 9)
Recombinase recognition sequences attB Pa01
cttcgagaccgtgacctacatgctcgaagggcgtatgcgccacgaaga (SEQ ID NO: 10)
Si74 attB
ttccgacgcagtttccgacgagtacgaggacgaggacagacgtgcctaccggcaaggtcaagtggttcaacagcgagaagggcttc
ggctttctctccc gcgacgacggcgg (SEQ ID NO: 11)
PhiC31 attB
Ccccaactggggtaacctttgagttctctcagttggggg (SEQ ID NO: 12)
Bxb1 attB
ggcttgtcgacgacggcggtctccgtcgtcaggatcat (SEQ ID NO: 13)
Pa01 attP
attctcatatccatcttgagtcttctttctcgcaagacaacacgaaatagacacagtctc (SEQ ID NO: 14)
Si74 attP
tagtgacgtctgtccgcgcagtgatcgagggagtgtgtgctttgccgactggcaaggtcaagccggtctgctaggcacagagagccg
gtacagtcctccccatgcaacccaa (SEQ ID NO: 15)
PhiC31 attP
gtgccagggcgtgcccttgggctccccgggcgcg (SEQ ID NO: 16)
Bxb1 attP
gtggtttgtctggtcaaccaccgcggtctcagtggtgtacggtacaaaccca (SEQ ID NO: 17)
WPRE
aatcaacctctggattacaaaatttgtgaaagattgactggtattcttaactatgttgctccttttacgctatgtggatacgctgctttaatgc
ctttgtatcatgctattgcttcccgtatggctttcattttctcctccttgtataaatcctggttgctgtctctttatgaggagttgtggcccgtt
gtcaggcaacgtggcgtggtgtgcactgtgtttgctgacgcaacccccactggttggggcattgccaccacctgtcagctcctttccgggacttt
cgctttccccctccctattgccacggcggaactcatcgccgcctgccttgcccgctgctggacaggggctcggctgttgggcactgaca
attccgtggtgttgtcggggaaatcatcgtcctttccttggctgctcgcctgtgttgccacctggattctgcgcgggacgtccttctgctac
gtcccttcggccctcaatccagcggaccttccttcccgcggcctgc
tgccggctctgcggcctcttccgcgtcttcgccttcgccctcagacgagtcggatctccctttgggccgcctccccgc (SEQ ID
NO: 18)
Polyadenylation Sequences SV40 pA
aacttgtttattgcagcttataatggttacaaataaagcaatagcatcacaaatttcacaaataaagcatttttttcactgcattctagttgtgg
tttgtccaaactcatcaatgtatctta (SEQ ID NO: 10)
bGH pA
ctgtgccttctagttgccagccatctgttgtttgcccctcccccgtgccttccttgaccctggaaggtgccactcccactgtcctttcctaata
aaatgaggaaattgcatcgcattgtctgagtaggtgtcattctattctggggggtggggggggcaggacagcaagggggaggattgg
gaagacaatagcaggcatgctggggatgcggtgggctctatgg (SEQ ID NO: 20)
β-globin pA
gctcgctttcttgctgtccaatttctattaaaggttcctttgttccctaagtccaactactaaactgggggatattatgaagggccttgagcatc
tggattctgcctaataaaaaacatttattttcattgcaatgatgtatttaaattatttctgaatattttactaaaaagggaatgtgggaggtcag
tgcatttaaaacataaagaaatgaagagctagttcaaaccttgggaaaatacactatatcttaaactccatgaaagaaggtgaggctgcaa
acagctaatgcacattggcaacagcccctgatgcctatgccttattcatccctcagaaaaggattcaagtagaggcttgatttggaggtta
aagttttgctatgctgtatttta (SEQ ID NO: 21)
HSV TK pA
cggcaataaaaagacagaataaaacgcacgggtgttgggtcgtttgttc (SEQ ID NO: 22)
Fluorescent Proteins
Fluorescent protein (FP)StayGold nucleic acid sequence
atggcttctacgccatttaaattccaattgaaaggcactatcaacggcaaatcttttactgtggaaggggagggggagggtaactcacat
gaaggctcccataaaggaaaatatgtctgcacatccgggaagctccctatgtcttgggcagctctgggtacctcattcgggtacggcat
gaaatactacaccaaatatccgagtggattgaagaattggttccacgaggtaatgccagagggcttcacatacgaccgccacatccaat
ataaaggagatggctctatacatgctaagcatcaacacttcatgaagaatggtacttatcataacattgttgaattcacaggccaggatttc
aaagaaaactctccggtacttaccggggacatgaatgtaagcttgccgaacgaagttcagcatatacccagagatgacggggtagaat
gcccggtcactctgctgtatcccttgttgtcagacaaaagtaaatgtgttgaagcacaccagaacacaatatgtaaaccgctccataatca
gcccgcgcctgatgtcccataccattggatacgcaagcagtatacacaaagtaaagatgacacggaggaacgggatcatatatgtcag
tccgagacactggaggcacacctc (SEQ ID NO: 23)
Fluorescent protein (FP)StayGold amino acid sequence
MASTPFKFQLKGTINGKSFTVEGEGEGNSHEGSHKGKYVCTSGKLPMSWAALGTSF
GYGMKYYTKYPSGLKNWFHEVMPEGFTYDRHIQYKGDGSIHAKHQHFMKNGTYH
NIVEFTGQDFKENSPVLTGDMNVSLPNEVQHIPRDDGVECPVTL
LYPLLSDKSKCVEAHQNTICKPLHNQPAPDVPYHWIRKQYTQSKDDTEERDHICQSE
TLEAHL (SEQ ID NO: 24)
Fluorescent protein (FP)mScarlet3
MDSTEAVIKEFMRFKVHMEGSMNGHEFEIEGEGEGRPYEGTQTAKLRVTKGGPLPFSWDILS
PQFMYGSRAFTKHPADIPDYWKQSFPEGFKWERVMNFEDGGAVSVAQDTSLEDGTLIYKVK
LRGTNFPPDGPVMQKKTMGWEASTERLYPEDVVLKGDIKMALRLKDGGRYLADFKTTYRA
KKPVQMPGAFNIDRKLDITSHNEDYTVVEQYERSVARHSTGGSGGS (SEQ ID NO: 25)
Fluorescent protein (FP)ZsGreen
MAQSKHGLTKEMTMKYRMEGCVDGHKFVITGEGIGYPFKGKQAINLCVVEGGPLP
FAEDILSAAFNYGNRVFTEYPQDIVDYFKNSCPAGYTWDRSFLFEDGAVCICNADIT
VSVEENCMYHESKFYGVNFPADGPVMKKMTDNWEPSCEKIIPVPKQGILKGDVSMY
LLLKDGGRLRCQFDTVYKAKSVPRKMPDWHFIQHKLTREDRSDAKNQKWHLTEHA
IASGSALP (SEQ ID NO: 26)
Fluorescent protein (FP)mNeonGreen
MVSKGEEDNMASLPATHELHIFGSINGVDFDMVGQGTGNPNDGYEELNLKSTKGDL
QFSPWILVPHIGYGFHQYLPYPDGMSPFQAAMVDGSGYQVHRTMQFEDGASLTVN
YRYTYEGSHIKGEAQVKGTGFPADGPVMTNSLTAADWCRSKKTYPNDKTIISTFKW
SYTTGNGKRYRSTARTTYTFAKPMAANYLKNQPMYVFRKTELKHSKTELNFKEWQ
KAFTDVMG MDELYK (SEQ ID NO: 27)
Fluorescent protein (FP)mClover3
MVSKGEELFTGVVPILVELDGDVNGHKFSVRGEGEGDATNGKLTLKFICTTGKLPVP
WPTLVTTFGYGVACFSRYPDHMKQHDFFKSAMPEGYVQERTISFKDDGTYKTRAEV
KFEGDTLVNRIELKGIDFKEDGNILGHKLEYNFNSHYVYITADKQKNCIKANFKIRHN
VEDGSVQLADHYQQNTPIGDGPVLLPDNHYLSHQSKLSKDPNEKRDHMVLLEFVTA
AGITHGMDELYK (SEQ ID NO: 28)
Fluorescent protein (FP)EGFP
MVSKGEELFTGVVPILVELDGDVNGHKFSVSGEGEGDATYGKLTLKFICTTGKLPVPWPTLV
TTLTYGVQCFSRYPDHMKQHDFFKSAMPEGYVQERTIFFKDDGNYKTRAEVKFEGDTLVNR
IELKGIDFKEDGNILGHKLEYNYNSHNVYIMADKQKNGIKVNFKIRHNIEDGSVQLADHYQQ
NTPIGDGPVLLPDNHYLSTQSALSKDPNEKRDHMVLLEFVTAAGITLGMDELYK (SEQ ID
NO: 29)
Fluorescent protein (FP)mCherry2
MVSKGEEDNMAIIKEFMRFKVHMEGSVNGHEFEIEGEGEGRPYEGTQTAKLKVTKG
GPLPFAWDILSPQFMYGSKAYVKHPADIPDYLKLSFPEGFNWERVMNFEDGGVVTV
TQDSSLQDGEFIYKVKLRGTNFPSDGPVMQCRTMGWEASTERMYPEDGALKGEIKQ
RLKLKDGGHYDAEVKTTYKAKKPVQLPGAYNVDIKLDILSHNEDYTIVEQYERAEG
RHSTGGMD ELYK (SEQ ID NO: 30)
Fluorescent protein (FP)mTagBFP2
MVSKGEELIKENMHMKLYMEGTVDNHHFKCTSEGEGKPYEGTQTMRIKVVEGGPL
PFAFDILATSFLYGSKTFINHTQGIPDFFKQSFPEGFTWERVTTYEDGGVLTATQDTSL
QDGCLIYNVKIRGVNFTSNGPVMQKKTLGWEAFTETLYPADGGLEGRNDMALKLV
GGSHLIANAKTTYRSKKPAKNLKMPGVYYVDYRLERIKEANNETYVEQHEVAVAR
YCDLPSKLGH KLN (SEQ ID NO: 31)
Fluorescent protein (FP)Venus
MVSKGEELFTGVVPILVELDGDVNGHKFSVSGEGEGDATYGKLTLKLICTTGKLPVPWPTLV
TTLGYGLQCFARYPDHMKQHDFFKSAMPEGYVQERTIFFKDDGNYKTRAEVKFEGDTLVNR
IELKGIDFKEDGNILGHKLEYNYNSHNVYITADKQKNGIKANFKIRHNIEDGGVQLADHYQQ
NTPIGDGPVLLPDNHYLSYQSALSKDPNEKRDHMVLLEFVTAAGITLGMDELYK (SEQ ID
NO: 32)
Fluorescent protein (FP)mCerulean
MVSKGEELFTGVVPILVELDGDVNGHKFSVSGEGEGDATYGKLTLKFICTTGKLPVP
WPTLVTTLTWGVQCFARYPDHMKQHDFFKSAMPEGYVQERTIFFKDDGNYKTRAE
VKFEGDTLVNRIELKGIDFKEDGNILGHKLEYNAISDNVYITADKQKNGIKANFKIRH
NIEDGSVQLADHYQQNTPIGDGPVLLPDNHYLSTQSKLSKDPNEKRDHMVLLEFVT
AAGITLGMDELYK (SEQ ID NO: 33)
Fluorescent protein (FP)emiRFP670
MAEGSVARQPDLLTCEHEEIHLAGSIQPHGALLVVSEHDHRVIQASANAAEFLNLGSVLGVP
LAEIDGDLLIKILPHLDPTAEGMPVAVRCRIGNPSTEYCGLMHRPPEGGLIIELERAGPSIDLSG
TLAPALERIRTAGSLRALCDDTVLLFQQCTGYDRVMVYRFDEQGHGLVFSECHVPGLESYFG
NRYPSSTVPQMARQLYVRQRVRVLVDVTYQPVPLEPRLSPLTGRDLDMSGCFLRSMSPCHL
QFLKDMGVRATLAVSLVVGGKLWGLVVCHHYLPRFIRFELRAICKRLAERIATRITALES
(SEQ ID NO: 34)
Large Serine Recombinases
Pa01 nucleic acid
atgggccctagcgccttcagctacgtgcgctttagcagcggcaagcaggccaagggcagctctgaacacagacagagagccatgct
gggacagtggctggaacagcaccctagcttcacactgagtgatctgcggttcgaggacctgggcagaagcggattcagcggcgagc
acctggatcacggcctgggccagctgctggctgctatcgacagcggcgccatcaagagcggagatgtgatcctggtcgaggccgtg
gacagaatcggcagactggagcctctggaaatgctgcctctgttctccagaatcgtgaaggccggcgtgtccgttatcaccctggaag
atggccacgtttacgaccggagctccgtgaacgagacatctctgtttctgctggtggccaaaatccagcaggcccacgagtactctaat
agactgtctcgaagaatcaacgcctcttacaccgctcggagagaaaaagctaaagcaggcctgggcatcaagagagagacacctgt
gtggctgaccacagacggccaactggtgcctcacgtggcccctcacatcgcccaggcttttcaggactacgccgatggcctgggaga
gagaaggatttgtagaaagctgcgggagagcggcctggaagaattctccaagaccaacgccaccaccgtgcggagatggctgaag
aaccggaccgccattggatattggaacgacatccctgatgtgtaccctcatgtcgtggaccctgctctgttctaccaggtgcagcagcgc
ctggacgcccctaaggtggacagagccaagcccagcgctcactacctgacaggcctcgtgaagtgcgccgtgtgcggcagaaacta
caactacaaacagcggaagcacaccgaccccgctatgctgtgcaccagcagagccagactggccggggaaggctgcagcaatag
caagacataccccgtgatagttctggaccaggtgcggaagctgaccagcctgccgttcctgcaacacgccatggaatccgcctcatct
caggccgacccaagcagccagagactggccgtgatcgacggcgaaatcggcgagctgtccagaaagatcagcgaggccacaaag
gccctgctggtgctgggcttcacccctgagatccaggagagcctggaacagctcaagaccgccagagaagccctggaggaagaaa
gagccaccctgctgctgcctcaggccgagaagctgacaacagctcagctggaggccttcagcaacggcctgctggacgacgagcc
catgaagctcaaccacgtgcttcagaccgccggctacagcatggtggtgcaccccgacggctctatcgacgtggatggaaaaagatt
cgtgtacgagggcgctagccggaaagagaaagtgtacaagctgagactgatcggcgaggataagcagtggagcctgcccatcctga
ccccacagatggccacttacaagtccctgttcatggccgctgtgcggctgcctggcgaccccagcgaggaagagctgagacggttcg
aggaagccaagcacagcgagcgg (SEQ ID NO: 35)
Pa01 amino acid
MGPSAFSYVRFSSGKQAKGSSEHRQRAMLGQWLEQHPSFTLSDLRFEDLGRSGFSG
EHLDHGLGQLLAAIDSGAIKSGDVILVEAVDRIGRLEPLEMLPLFSRIVKAGVSVITLE
DGHVYDRSSVNETSLFLLVAKIQQAHEYSNRLSRRINASYTARREKAKAGLGIKRET
PVWLTTDGQLVPHVAPHIAQAFQDYADGLGERRICRKLRESGLEEFSKTNATTVRR
WLKNRTAIGYWNDIPDVYPHVVDPALFYQVQQRLDAPKVDRAKPSAHYLTGLVKC
AVCGRNYNYKQRKHTDPAMLCTSRARLAGEGCSNSKTYPVIVLDQVRKLTSLPFLQ
HAMESASSQADPSSQRLAVIDGEIGELSRKISEATKALLVLGFTPEIQESLEQLKTARE
ALEEERATLLLPQAEKLTTAQLEAFSNGLLDDEPMKLNHVLQTAGYSMVVHPDGSI
DVDGKRFVYEGASRKEKVYKLRLIGEDKQWSLPILTPQMATYKSLFMAAVRLPGDP
SEEELRRFEEAKHSER (SEQ ID NO: 36)
Si74
MQPNLRYLACLRLSADSDGSTSIEWQRGVIRHHVSSPHLSGVLVGEAEDTDVSGSLS
PFKRPKLGKWLTAKADEFDVIIAAKMDRLTRRSMHFNELLEWAQQNGKFIVCVEEG
FDLSTPQGKMMARMTAVFAEAEWDTIQARILNGVQTRLENRSWLVGAPPTGYRIKT
VEGGKRKILEIDQDFYPYVEEIFRRIREGQSTHRIARDENGRSVLTWGDHLRKLKGEE
PKGTQWQATIINKFIRSSWVPGLYTYKGEAVLDDQGDPVILPETPLATMDEWTDLV
DRIKPAPKPEGATGGSRNSAKSLLSGVAHCGECGAPFTSLMDSGYKRKDGTKVPGH
RRYRCSNKFKGGDCKNGSYVRADVLDSWVDQAIRDSIGQEDMYERAGKGPSQARE
LQETKARLAKLEADYESGKYDGEGQDESYWRMNKNLSAKVAHLAKQEAERANPT
FKATGKKYGEVWEAKDQEDRRDFLRTYGVKVFVWGEGADKKDRGYAMNLGDIK
TMAEELFPNRDRARFKLVHTHNAPEGYLSKIGIAVGLLKYGHP LEVKLRSPENS
(SEQ ID NO: 37)
PhiC31
MDTYAGAYDRQSRERENSSAASPATQRSANEDKAADLQREVERDGGRFRFVGHFSEAPGTS
AFGTAERPEFERILNECRAGRLNMIIVYDVSRFSRLKVMDAIPIVSELLALGVTIVSTQEGVFR
QGNVMDLIHLIMRLDASHKESSLKSAKILDTKNLQRELGGYVGGKAPYGFELVSETKEITRN
GRMVNVVINKLAHSTTPLTGPFEFEPDVIRWWWREIKTHKHLPFKPGSQAAIHPGSITGLCKR
MDADAVPTRGETIGKKTASSAWDPATVMRILRDPRIAGFAAEVIYKKKPDGTPTTKIEGYRIQ
RDPITLRPVELDCGPIIEPAEWYELQAWLDGRGRGKGLSRGQAILSAMDKLYCECGAVMTSK
RGEESIKDSYRCRRRKVVDPSAPGQHEGTCNVSMAALDKFVAERIFNKIRHAEGDEETLALL
WEAARRFGKLTEAPEKSGERANLVAERADALNALEELYEDRAAGAYDGPVGRKHFRKQQA
ALTLRQQGAEERLAELEAAEAPKLPLDQWFPEDADADPTGPKSWWGRASVDDKR
VFVGLFVDKIVVTKSTTGRGQGTPIEKRASITWAKPPTDDDEDDAQDGTEDVA
(SEQ ID NO: 38)
Bxb1
MRALVVIRLSRVTDATTSPERQLESCQQLCAQRGWDVVGVAEDLDVSGAVDPFDR
KRRPNLARWLAFEEQPFDVIVAYRVDRLTRSIRHLQQLVHWAEDHKKLVVSATEAH
FDTTTPFAAVVIALMGTVAQMELEAIKERNRSAAHFNIRAGKYRGSLPPWGYLPTRV
DGEWRLVPDPVQRERILEVYHRVVDNHEPLHLVAHDLNRRGVLSPKDYFAQLQGR
EPQGREWSATALKRSMISEAMLGYATLNGKTVRDDDGAPLVRAEPILTREQLEALR
AELVKTSRAKPAVSTPSLLLRVLFCAVCGEPAYKFAGGGRKHPRYRCRSMGFPKHC
GNGTVAMAEWDAFCEEQVLDLLGDAERLEKVWVAGSDSAVELAEVNAELVDLTS
LIGSPAYRAGSPQREALDARIAALAARQEELEGLEARPSGWEWRETGQRFGDWWRE
QDTAAKNTWLRSMNVRLTF DVRGGLTRTIDFGDLQEYEQHLRLGSVVERLHTGMS
(SEQ ID NO: 39)
Antibiotic Resistance Genes and Proteins (an Embodiment of Negative Selection Element)
Puromycin
MTEYKPTVRLATRDDVPRAVRTLAAAFADYPATRHTVDPDRHIERVTELQELFLTR
VGLDIGKVWVADDGAAVAVWTTPESVEAGAVFAEIGPRMAELSGSRLAAQQQME
GLLAPHRPKEPAWFLATVGVSPDHQGKGLGSAVVLPGVEAAERAGVPAFLETSAPR
NLPFYERLGFTVTADVEVPEGPRTWCMTRKPGA (SEQ ID NO: 40)
Blasticidin Nucleic Acid Sequence
atggcgaagcccctgtcacaggaggagagtaccctgattgaacgggcgacagcgacaataaacagcatcccgattagtgaggattac
tccgtcgcatcagcagcactgagttctgatggtaggatcttcaccggggtgaacgtctaccacttcacggggggaccatgtgcagagtt
ggtcgttttgggaaccgcagcagcagcggcggctggtaatctgacttgtatagtagccatcggaaatgaaaaccgcggcattctgtccc
cgtgcgggcgctgcaggcaggttttgctggatctgcatcccgggataaaagctattgtaaaagattcagacggccagccaacggccgt
tggcatcagggagcttcttccatccggttatgtgtgggagggc (SEQ ID NO: 41)
Blasticidin Amino acid Sequence
MAKPLSQEESTLIERATATINSIPISEDYSVASAALSSDGRIFTGVNVYHFTGGPCAEL
VVLGTAAAAAAGNLTCIVAIGNENRGILSPCGRCRQVLLDLHPGIKAIVKDSDGQPT
AVGIRELLPSGYVWEG (SEQ ID NO: 42)
Hygromycin
MKKPELTATSVEKFLIEKFDSVSDLMQLSEGEESRAFSFDVGGRGYVLRVNSCADGF
YKDRYVYRHFASAALPIPEVLDIGEFSESLTYCISRRAQGVTLQDLPETELPAVLQPV
AEAMDAIAAADLSQTSGFGPFGPQGIGQYTTWRDFICAIADPHVYHWQTVMDDTVS
ASVAQALDELMLWAEDCPEVRHLVHADFGSNNVLTDNGRITAVIDWSEAMFGDSQ
YEVANIFFWRPWLACMEQQTRYFERRHPELAGSPRLRAYMLRIGLDQLYQSLVDGN
FDDAAWAQGRCDAIVRSGAGTVGRTQIARRSAAVWTDGCVEVLADSGNRRPSTRP
RAKE (SEQ ID NO: 43)
G418
MGSAIEQDGLHAGSPAAWVERLFGYDWAQQTIGCSDAAVFRLSAQGRPVLFVKTDLSGALN
ELQDEAARLSWLATTGVPCAAVLDVVTEAGRDWLLLGEVPGQDLLSSHLAPAEKVSIMAD
AMRRLHTLDPATCPFDHQAKHRIERARTRMEAGLVDQDDLDEEHQGLAPAELFARLKARM
PDGDDLVVTHGDACLPNIMVENGRFSGFIDCGRLGVADRYQDIALATRD
IAEELGGEWADRFLVLYGIAAPDSQRIAFYRLLDEFF (SEQ ID NO: 44)
Additional Negative Selection Gene or Protein
Double inducible caspase-9 nucleic acid sequence
atgatcagtctgattgcggcgttagcggtagatcgcgttatcggcatggaaaacgccatgccgtggaacctgcctgccgatctcgcctg
gtttaaacgcaacaccttaaataaacccgtgattatgggccgccatacctgggaatcaatcggtcgtccgttgccaggacgcaaaaatat
tatcctcagcagtcaaccgggtacggacgatcgcgtaacgcgcgtgaagtcggtggatgaagccatcgcggcgtgtggtgacgtacc
agaaatcatggtgattggcggcggtcgcgtttatgaacagttcttgccaaaagcgcaaaaactgtatctgagccatatcgacgcagaag
tggacggcgacacccatttcccggattacgagccggatgactgggaatcggtattcagcgaattccacgatgctgatgcgctgaactct
cacagctattgctttgagattctggagcggcgaagtggatctgagacaccaggcacgtctgagtctgcaactccagagtccggagtgc
aggtggagactatctccccaggagacgggcgcaccttccccaagcgcggccagacctgcgtggtgcactacaccgggatgcttgaa
gatggaaagaaagttgattcctcccgggacagaaacaagccctttaagtttatgctaggcaagcaggaggtgatccgaggctgggaa
gaaggggttgcccagatgagtgtgggtcagagagccaaactgactatatctccagattatgcctatggtgccactgggcacccaggca
tcatcccaccacatgccactctcgtcttcgatgtggagcttctaaaactggaatctggcggtggatccggagtcgacggatttggtgatgt
cggtgctcttgagagtttgaggggaaatgcagatttggcttacatcctgagcatggagccctgtggccactgcctcattatcaacaatgtg
aacttctgccgtgagtccgggctccgcacccgcactggctccaacatcgactgtgagaagttgcggcgtcgcttctcctcgctgcatttc
atggtggaggtgaagggcgacctgactgccaagaaaatggtgctggctttgctggagctggcgcggcaggaccacggtgctctgga
ctgctgcgtggtggtcattctctctcacggctgtcaggccagccacctgcagttcccaggggctgtctacggcacagatggatgccctg
tgtcggtcgagaagattgtgaacatcttcaatgggaccagctgccccagcctgggagggaagcccaagctctttttcatccaggcctgt
ggtggggagcagaaagaccatgggtttgaggtggcctccacttcccctgaagacgagtcccctggcagtaaccccgagccagatgc
caccccgttccaggaaggtttgaggaccttcgaccagctggacgccatatctagtttgcccacacccagtgacatctttgtgtcctactct
actttcccaggttttgtttcctggagggaccccaagagtggctcctggtacgttgagaccctggacgacatctttgagcagtgggctcact
ctgaagacctgcagtccctcctgcttagggtcgctaatgctgtttcggtgaaagggatttataaacagatgcctggttgctttaatttcctcc
ggaaaaaacttttctttaaaacatca (SEQ ID NO: 45)
Double inducible caspase-9 amino acid sequence
MISLIAALAVDRVIGMENAMPWNLPADLAWFKRNTLNKPVIMGRHTWESIGRPLPG
RKNIILSSQPGTDDRVTRVKSVDEAIAACGDVPEIMVIGGGRVYEQFLPKAQKLYLS
HIDAEVDGDTHFPDYEPDDWESVFSEFHDADALNSHSYCFEILERRSGSETPGTSESA
TPESGVQVETISPGDGRTFPKRGQTCVVHYTGMLEDGKKVDSSRDRNKPFKFMLGK
QEVIRGWEEGVAQMSVGQRAKLTISPDYAYGATGHPGIIPPHATLVFDVELLKLESG
GGSGVDGFGDVGALESLRGNADLAYILSMEPCGHCLIINNVNFCRESGLRTRTGSNI
DCEKLRRRFSSLHFMVEVKGDLTAKKMVLALLELARQDHGALDCCVVVILSHGCQ
ASHLQFPGAVYGTDGCPVSVEKIVNIFNGTSCPSLGGKPKLFFIQACGGEQKDHGFE
VASTSPEDESPGSNPEPDATPFQEGLRTFDQLDAISSLPTPSDIFVSYSTFPGFVSWRDP
KSGSWYVETLDDIFEQWAHSEDLQSLLLRVANAVSVKGIYKQ
MPGCFNFLRKKLFFKTS (SEQ ID NO: 46)
Herpes simplex virus thymidine kinase
MASYPCHQHASAFDQAARSRGHSNRRTALRPRRQQEATEVRLEQKMPTLLRVYIDG
PHGMGKTTTTQLLVALGSRDDIVYVPEPMTYWQVLGASETIANIYTTQHRLDQGEIS
AGDAAVVMTSAQITMGMPYAVTDAVLAPHIGGEAGSSHAPPPALTLIFDRHPIAALL
CYPAARYLMGSMTPQAVLAFVALIPPTLPGTNIVLGALPEDRHIDRLAKRQRPGERL
DLAMLAAIRRVYGLLANTVRYLQGGGSWREDWGQLSGTAVPPQGAEPQSNAGPRP
HIGDTLFTLFRAPELLAPNGDLYNVFAWALDVLAKRLRPMHVFILDYDQSPAGCRD
ALLQLTSGMVQTHVTTPGSIPTICDLARTFAREMGEAN* (SEQ ID NO: 47)

| SEQ ID | | |
|---|---|---|
| NO: | Component | Variant |
| 1 | Long Terminal Repeat (LTR) | Lentiviral LTR |
| 2 | Inverted terminal repeat (ITR) | Adeno-associated virus ITR |
| 3 | Promoter | CMV |
| 4 | Promoter | EF1a |
| 5 | Promoter | SFFV |
| 6 | Promoter | CAG |
| 7 | Promoter | CBh |
| 8 | Promoter | RSV |
| 9 | Promoter | MSCV |
| 10 | attB | Pa01 attB |
| 11 | attB | Si74 attB |
| 12 | attB | PhiC31 attB |
| 13 | attB | Bxb1 attB |
| 14 | attP | Pa01 attP |
| 15 | attP | Si74 attP |
| 16 | attP | PhiC31 attP |
| 17 | attP | Bxb1 attP |
| 18 | WPRE | WPRE |
| 19 | Polyadenylation (pA) | SV40 pA |
| 20 | Polyadenylation (pA) | bGH pA |
| 21 | Polyadenylation (pA) | β-globin pA |
| 22 | Polyadenylation (pA) | HSV TK pA |
| 23 | Fluorescent protein (FP) | StayGold nucleic acid sequence |
| 24 | Fluorescent protein (FP) | StayGold amino acid sequence |
| 25 | Fluorescent protein (FP) | mScarlet3 |
| 26 | Fluorescent protein (FP) | ZsGreen |
| 27 | Fluorescent protein (FP) | mNeonGreen |
| 28 | Fluorescent protein (FP) | mClover3 |
| 29 | Fluorescent protein (FP) | EGFP |
| 30 | Fluorescent protein (FP) | mCherry2 |
| 31 | Fluorescent protein (FP) | mTagBFP2 |
| 32 | Fluorescent protein (FP) | Venus |
| 33 | Fluorescent protein (FP) | mCerulean |
| 34 | Fluorescent protein (FP) | emiRFP670 |
| 35 | Large serine recombinase | Pa01 nucleic acid sequence |
| | (LSR) | |
| 36 | Large serine recombinase | Pa01 amino acid sequence |
| | (LSR) | |
| 37 | Large serine recombinase | Si74 |
| | (LSR) | |
| 38 | Large serine recombinase | PhiC31 |
| | (LSR) | |
| 39 | Large serine recombinase | Bxb1 |
| | (LSR) | |
| 40 | Antibiotic resistance gene | Puromycin |
| | (ARG) | |
| 41 | Antibiotic resistance gene | Blasticidin nucleic acid sequence |
| | (ARG) | |
| 42 | Antibiotic resistance gene | Blasticidin amino acid sequence |
| | (ARG) | |
| 43 | Antibiotic resistance gene | Hygromycin |
| | (ARG) | |
| 44 | Antibiotic resistance gene | G418 |
| | (ARG) | |
| 45 | Negative selection gene | Double inducible caspase-9 |
| | (NSG) | NA seq |
| 46 | Negative selection gene | Double inducible caspase-9 |
| | (NSG) | AA seq |
| 47 | Negative selection gene | Herpes simplex virus thymidine |
| | (NSG) | kinase |

In some embodiments, the disclosure relates to a composition comprising one or more nucleic acid sequences; one or more nucleic acid molecules comprising those nucleic acid sequences; and compositions comprising cells having a transduction targeting element comprising one or more nucleic acid sequences disclosed herein. In some embodiments, the nucleic acid molecule is a plasmid, cosmid, or endogenous viral genome packaged within a viral vector. In some embodiments, the composition comprises a viral vector, such as a lentivirus or an AAV particle comprising the nucleic acid molecule. In some embodiments, the composition comprises a lentivirus or an AAV particle comprising a first and second nucleic acid molecule, the first nucleic acid molecule is a single-stranded positive nucleic acid sequence and the second nucleic acid molecule is a single-stranded negative strand nucleic acid sequence. In some embodiments, the cells are stably integrated with heterologous DNA comprising:
(a) a first expressible nucleic acid and a second expressible nucleic acid;
(b) a first regulatory sequence operably linked to the first expressible nucleic acid;
(c) a second regulatory sequence operably linked to the second expressible nucleic acid; and
(d) a recombinase element encoding a serine recombinase positioned on either the first or the second expressible nucleic acid;

wherein (a), (b), (c) and (d) are positioned between a 5′ viral packaging sequence and a 3′ viral packaging sequence. In some embodiments, the cells are stably integrated with heterologous DNA comprising:
(c) a first expressible nucleic acid and a second expressible nucleic acid;
(f) a first regulatory sequence operably linked to the first expressible nucleic acid;
(g) a second regulatory sequence operably linked to the second expressible nucleic acid; and
(h) a recombinase element encoding a serine recombinase positioned on either the first or the second expressible nucleic acid;
wherein (a), (b), (c) and (d) are positioned between a 5′ lentiviral LTR and a 3′ lentiviral LTR. In some embodiments, the cells are stably integrated with heterologous DNA comprising:
(a) a first expressible nucleic acid and a second expressible nucleic acid;
(b) a first regulatory sequence operably linked to the first expressible nucleic acid;
(c) a second regulatory sequence operably linked to the second expressible nucleic acid; and
(d) a recombinase element encoding a serine recombinase positioned on either the first or the second expressible nucleic acid;
wherein (a), (b), (c) and (d) are positioned between a 5′ AAV ITR and a 3′ lentiviral ITR.

In some embodiments the nucleic acid sequence or cells comprising the nucleic acid sequences disclosed herein comprise a transduction targeting element comprising:
(a) a first expressible nucleic acid and a second expressible nucleic acid;
(b) a first regulatory sequence operably linked to the first expressible nucleic acid;
(c) a second regulatory sequence operably linked to the second expressible nucleic acid; and
(d) a recombinase element encoding a serine recombinase positioned on either the first or the second expressible nucleic acid;

wherein (a), (b), (c) and (d) are positioned between a 5′ viral tandem repeat sequence and a 3′ viral tandem repeat sequence. In some embodiments, the recombinase element encodes a serine recombinase. In some embodiments, the recombinase element encodes a large serine recombinase. In some embodiments, the first and/or second expressible nucleic acid encodes a targeting protein, or label to track the presence of the first or second nucleic acid sequence by exposure of the cell to a stimulus or probe.

In some embodiments the nucleic acid sequence or cells comprising the nucleic acid sequences disclosed herein comprise a viral tandem repeat sequence, or viral packaging sequence at one or both ends of the nucleic acid sequence. In some embodiments the nucleic acid sequence or cells comprising the nucleic acid sequences disclosed herein comprise SEQ ID NO:1 or a functional fragment thereof comprising about 70%, 75%, 80%, 85%, 90%, 91%, 9% 2, 93%, 94%, 95%, 96%, 97%, 98%, or 99% sequence identity to SEQ ID NO:1. In some embodiments the nucleic acid sequence or cells comprising the nucleic acid sequences disclosed herein comprise SEQ ID NO:2 or a functional fragment thereof comprising about 70%, 75%, 80%, 85%, 90%, 91%, 9% 2, 93%, 94%, 95%, 96%, 97%, 98%, or 99% sequence identity to SEQ ID NO:2.

In some embodiments, the nucleic acid sequence or cells comprising the nucleic acid sequences disclosed herein comprise a regulatory sequence that is operably linked to the first and/or second expressible nucleic acid sequence. In some embodiments the nucleic acid sequence or cells comprising the nucleic acid sequences disclosed herein comprise a regulatory sequence that SEQ ID NO: 3 or a functional fragment thereof comprising about 70%, 75%, 80%, 85%, 90%, 91%, 9% 2, 93%, 94%, 95%, 96%, 97%, 98%, or 99% sequence identity to SEQ ID NO:3. In some embodiments the nucleic acid sequence or cells comprising the nucleic acid sequences disclosed herein comprise a regulatory sequence that SEQ ID NO:4 or a functional fragment thereof comprising about 70%, 75%, 80%, 85%, 90%, 91%, 9% 2, 93%, 94%, 95%, 96%, 97%, 98%, or 99% sequence identity to SEQ ID NO:4. In some embodiments the nucleic acid sequence or cells comprising the nucleic acid sequences disclosed herein comprise a regulatory sequence that SEQ ID NO: 4 or a functional fragment thereof comprising about 70%, 75%, 80%, 85%, 90%, 91%, 9% 2, 93%, 94%, 95%, 96%, 97%, 98%, or 99% sequence identity to SEQ ID NO:4. In some embodiments the nucleic acid sequence or cells comprising the nucleic acid sequences disclosed herein comprise a regulatory sequence that SEQ ID NO:5 or a functional fragment thereof comprising about 70%, 75%, 80%, 85%, 90%, 91%, 9% 2, 93%, 94%, 95%, 96%, 97%, 98%, or 99% sequence identity to SEQ ID NO:5. In some embodiments the nucleic acid sequence or cells comprising the nucleic acid sequences disclosed herein comprise a regulatory sequence that SEQ ID NO: 6 or a functional fragment thereof comprising about 70%, 75%, 80%, 85%, 90%, 91%, 9% 2, 93%, 94%, 95%, 96%, 97%, 98%, or 99% sequence identity to SEQ ID NO:6. In some embodiments the nucleic acid sequence or cells comprising the nucleic acid sequences disclosed herein comprise a regulatory sequence that SEQ ID NO:7 or a functional fragment thereof comprising about 70%, 75%, 80%, 85%, 90%, 91%, 9% 2, 93%, 94%, 95%, 96%, 97%, 98%, or 99% sequence identity to SEQ ID NO:7. In some embodiments the nucleic acid sequence or cells comprising the nucleic acid sequences disclosed herein comprise a regulatory sequence that SEQ ID NO: 8 or a functional fragment thereof comprising about 70%, 75%, 80%, 85%, 90%, 91%, 9% 2, 93%, 94%, 95%, 96%, 97%, 98%, or 99% sequence identity to SEQ ID NO:8. In some embodiments the nucleic acid sequence or cells comprising the nucleic acid sequences disclosed herein comprise a regulatory sequence that SEQ ID NO:9 or a functional fragment thereof comprising about 70%, 75%, 80%, 85%, 90%, 91%, 9% 2, 93%, 94%, 95%, 96%, 97%, 98%, or 99% sequence identity to SEQ ID NO:9.

In some embodiments, the nucleic acid sequence or cells comprising the nucleic acid sequences disclosed herein comprise a transduction targeting element comprising a first and/or second expressible nucleic acid sequence and a first recombinase attachment element and a second recombinase attachment element. The recombinase attachment elements are those sequences flanking a payload sequence or targeting that are recognized by a recombinase in the process of a recombination event. In some embodiments, the nucleic acid sequence or cells comprising the nucleic acid sequences disclosed herein comprise a transduction targeting element comprising a first recombinase attachment element and/or a second recombinase attachment element comprising one or more of one or a combination of: SEQ ID NO: 11, SEQ ID NO: 12, SEQ ID NO: 13, SEQ ID NO: 14, SEQ ID NO:15, SEQ ID NO:16, SEQ ID NO:17, SEQ ID NO: 18, or functional fragments thereof. In some embodiments, the nucleic acid sequence or cells comprising the nucleic acid sequences disclosed herein comprise a transduction targeting element comprising a first and/or second expressible nucleic acid sequence and a first recombinase attachment element and a second recombinase attachment element, wherein the first and second recombinase attachment elements comprise one or a combination of: SEQ ID NO:10, SEQ ID NO:14, or variants of SEQ ID NO:10 comprising about 70%, 75%, 80%, 85%, 90%, 91%, 9% 2, 93%, 94%, 95%, 96%, 97%, 98%, or 99% sequence identity to SEQ ID NO: 10; or variants of SEQ ID NO: 14 comprising about 70%, 75%, 80%, 85%, 90%, 91%, 9% 2, 93%, 94%, 95%, 96%, 97%, 98%, or 99% sequence identity to SEQ ID NO:14. In some embodiments, the nucleic acid sequence or cells comprising the nucleic acid sequences disclosed herein comprise a transduction targeting element comprising a first and/or second expressible nucleic acid sequence and a first recombinase attachment element and a second recombinase attachment element, wherein the first and second recombinase attachment elements comprise one or a combination of: SEQ ID NO: 11, SEQ ID NO: 15, or variants of SEQ ID NO: 11 comprising about 70%, 75%, 80%, 85%, 90%, 91%, 9% 2, 93%, 94%, 95%, 96%, 97%, 98%, or 99% sequence identity to SEQ ID NO: 11; or variants of SEQ ID NO: 15 comprising about 70%, 75%, 80%, 85%, 90%, 91%, 9% 2, 93%, 94%, 95%, 96%, 97%, 98%, or 99% sequence identity to SEQ ID NO: 15. In some embodiments, the nucleic acid sequence or cells comprising the nucleic acid sequences disclosed herein comprise a transduction targeting element comprising a first and/or second expressible nucleic acid sequence and a first recombinase attachment element and a second recombinase attachment element, wherein the first and second recombinase attachment elements comprise one or a combination of: SEQ ID NO: 12, SEQ ID NO: 16, or variants of SEQ ID NO: 12 comprising about 70%, 75%, 80%, 85%, 90%, 91%, 9% 2, 93%, 94%, 95%, 96%, 97%, 98%, or 99% sequence identity to SEQ ID NO:12; or variants of SEQ ID NO:16 comprising about 70%, 75%, 80%, 85%, 90%, 91%, 9% 2, 93%, 94%, 95%, 96%, 97%, 98%, or 99% sequence identity to SEQ ID NO:16. In some embodiments, the first and second recombinase attachment in the transduction targeting sequence are identical and are chosen from: SEQ ID NO: 10 through SEQ ID NO:17, or functional variants thereof.

In some embodiments, the nucleic acid sequence or cells comprising the nucleic acid sequences disclosed herein comprise a transduction targeting element comprising a first and/or second expressible nucleic acid sequence and a first recombinase attachment element and a second recombinase attachment element, wherein the first and second recombinase attachment elements comprise SEQ ID NO: 13 and SEQ ID NO:17, or variants of SEQ ID NO: 13 comprising about 70%, 75%, 80%, 85%, 90%, 91%, 9% 2, 93%, 94%, 95%, 96%, 97%, 98%, or 99% sequence identity to SEQ ID NO: 13; or variants of SEQ ID NO: 17 comprising about 70%, 75%, 80%, 85%, 90%, 91%, 9% 2, 93%, 94%, 95%, 96%, 97%, 98%, or 99% sequence identity to SEQ ID NO:17.

The disclosure relates to a nucleic acid sequence or a cell comprising a heterologous nucleic acid sequence positioned within its endogenous DNA of the cell comprising a transduction targeting element, the transduction targeting element comprising one or two expressible nucleic acid sequences operably linked to respective first and/or second regulatory elements, a first and second recombinase attachment element and one or more nucleic acid sequences encoding a probe. In some embodiments, the transduction targeting sequence comprising SEQ ID NO:23, or variants of SEQ ID NO:23 comprising about 70%, 75%, 80%, 85%, 90%, 91%, 9% 2, 93%, 94%, 95%, 96%, 97%, 98%, or 99% sequence identity to SEQ ID NO:23. In some embodiments, the transduction targeting sequence comprising a nucleic acid sequence encoding SEQ ID NO:24 or variants of SEQ ID NO:24 comprising about 70%, 75%, 80%, 85%, 90%, 91%, 9% 2, 93%, 94%, 95%, 96%, 97%, 98%, or 99% sequence identity to SEQ ID NO:24. In some embodiments, the transduction targeting sequence comprising a nucleic acid sequence encoding SEQ ID NO:25 or variants of SEQ ID NO:25 comprising about 70%, 75%, 80%, 85%, 90%, 91%, 9% 2, 93%, 94%, 95%, 96%, 97%, 98%, or 99% sequence identity to SEQ ID NO:25. In some embodiments, the transduction targeting sequence comprising a nucleic acid sequence encoding SEQ ID NO:26 or variants of SEQ ID NO:26 comprising about 70%, 75%, 80%, 85%, 90%, 91%, 9% 2, 93%, 94%, 95%, 96%, 97%, 98%, or 99% sequence identity to SEQ ID NO:26. In some embodiments, the transduction targeting sequence comprising a nucleic acid sequence encoding SEQ ID NO:27 or variants of SEQ ID NO:27 comprising about 70%, 75%, 80%, 85%, 90%, 91, 92, 93, 94, 95, 96, 97, 98, or 99% sequence identity to SEQ ID NO:27. In some embodiments, the transduction targeting sequence comprising a nucleic acid sequence encoding SEQ ID NO:28 or variants of SEQ ID NO:28 comprising about 70%, 75%, 80%, 85%, 90%, 91%, 9% 2, 93%, 94%, 95%, 96%, 97%, 98%, or 99% sequence identity to SEQ ID NO:28. In some embodiments, the transduction targeting sequence comprising a nucleic acid sequence encoding SEQ ID NO:29 or variants of SEQ ID NO:29 comprising about 70%, 75%, 80%, 85%, 90%, 91%, 9% 2, 93%, 94%, 95%, 96%, 97%, 98%, or 99% sequence identity to SEQ ID NO:29. In some embodiments, the transduction targeting sequence comprising a nucleic acid sequence encoding SEQ ID NO:30 or variants of SEQ ID NO:30 comprising about 70%, 75%, 80%, 85%, 90%, 91%, 9% 2, 93%, 94%, 95%, 96%, 97%, 98%, or 99% sequence identity to SEQ ID NO:30. In some embodiments, the transduction targeting sequence comprising a nucleic acid sequence encoding SEQ ID NO:31 or variants of SEQ ID NO:31 comprising about 70%, 75%, 80%, 85%, 90%, 91%, 9% 2, 93%, 94%, 95%, 96%, 97%, 98%, or 99% sequence identity to SEQ ID NO:31. In some embodiments, the transduction targeting sequence comprising a nucleic acid sequence encoding SEQ ID NO:32 or variants of SEQ ID NO:32 comprising about 70%, 75%, 80%, 85%, 90%, 91%, 9% 2, 93%, 94%, 95%, 96%, 97%, 98%, or 99% sequence identity to SEQ ID NO:32. In some embodiments, the transduction targeting sequence comprising a nucleic acid sequence encoding SEQ ID NO:33 or variants of SEQ ID NO:33 comprising about 70%, 75%, 80%, 85%, 90%, 91%, 9% 2, 93%, 94%, 95%, 96%, 97%, 98%, or 99% sequence identity to SEQ ID NO:33. In some embodiments, the transduction targeting sequence comprising a nucleic acid sequence encoding SEQ ID NO:34 or variants of SEQ ID NO:34 comprising about 70%, 75%, 80%, 85%, 90%, 91%, 9% 2, 93%, 94%, 95%, 96%, 97%, 98%, or 99% sequence identity to SEQ ID NO:34.

The disclosure also relates to a nucleic acid sequence or a cell comprising a transduction targeting element comprising a posttranscriptional regulatory element, such as a WPRE. In some embodiments, the disclosure relates to a nucleic acid sequence or a cell comprising a heterologous nucleic acid sequence positioned within its endogenous DNA of the cell comprising a transduction targeting element, the transduction targeting element comprising SEQ ID NO:18 or a functional variant of SEQ ID NO: 18 comprising about 70%, 75%, 80%, 85%, 90%, 91%, 9% 2, 93%, 94%, 95%, 96%, 97%, 98%, or 99% sequence identity to SEQ ID NO: 18.

In some embodiments the nucleic acid sequence or cells comprising the nucleic acid sequences disclosed herein comprise a transduction targeting element comprising:
a first expressible nucleic acid and a second expressible nucleic acid;
a first regulatory sequence operably linked to the first expressible nucleic acid;
a second regulatory sequence operably linked to the second expressible nucleic acid; and
a recombinase element encoding a serine recombinase positioned on either the first or the second expressible nucleic acid; and one or a plurality of posttranscriptional regulatory elements, optionally positioned 3′ in frame from the first and/or the second expressible nucleic acid; wherein (a), (b), (c) and (d) are positioned between a 5′ viral tandem repeat sequence and a 3′ viral tandem repeat sequence. In some embodiments the one or a plurality of posttranscriptional regulatory elements is SEQ ID NO: 19 or functional variant of SEQ ID NO:19 comprising about 70%, 75%, 80%, 85%, 90%, 91%, 9% 2, 93%, 94%, 95%, 96%, 97%, 98%, or 99% sequence identity to SEQ ID NO:19. In some embodiments the one or a plurality of posttranscriptional regulatory elements is SEQ ID NO:20 or functional variant of SEQ ID NO:20 comprising about 70%, 75%, 80%, 85%, 90%, 91%, 9% 2, 93%, 94%, 95%, 96%, 97%, 98%, or 99% sequence identity to SEQ ID NO:20. In some embodiments the one or a plurality of posttranscriptional regulatory elements is SEQ ID NO:21 or functional variant of SEQ ID NO:21 comprising about 70%, 75%, 80%, 85%, 90%, 91%, 9% 2, 93%, 94%, 95%, 96%, 97%, 98%, or 99% sequence identity to SEQ ID NO:21.

In some embodiments the one or a plurality of posttranscriptional regulatory elements is SEQ ID NO:22 or functional variant of SEQ ID NO:22 comprising about 70%, 75%, 80%, 85%, 90%, 91%, 9% 2, 93%, 94%, 95%, 96%, 97%, 98%, or 99% sequence identity to SEQ ID NO:22.

In some embodiments the nucleic acid sequence or cells comprising the nucleic acid sequences disclosed herein comprise a transduction targeting element comprising a sequence encoding a recombinase. In some embodiments the nucleic acid sequence or cells comprising the nucleic acid sequences disclosed herein comprise a transduction targeting element comprising a sequence encoding a recombinase configured to bind to the first and/or second recombinases attachment element within the nucleic acid sequence upon expression in a cell. In some embodiments, the one or more sequence encoding a recombinase is SEQ ID NO: 35 or functional variant of SEQ ID NO:35 comprising about 70%, 75%, 80%, 85%, 90%, 91%, 9% 2, 93%, 94%, 95%, 96%, 97%, 98%, or 99% sequence identity to SEQ ID NO:35.

In some embodiments, the one or more sequence encoding a recombinase encodes SEQ ID NO: 36 or functional variant of SEQ ID NO:36 comprising about 70%, 75%, 80%, 85%, 90%, 91%, 9% 2, 93%, 94%, 95%, 96%, 97%, 98%, or 99% sequence identity to SEQ ID NO:36. In some embodiments, the one or more sequence encoding a recombinase encodes SEQ ID NO: 37 or functional variant of SEQ ID NO:37 comprising about 70%, 75%, 80%, 85%, 90%, 91%, 9% 2, 93%, 94%, 95%, 96%, 97%, 98%, or 99% sequence identity to SEQ ID NO:37. In some embodiments, the one or more sequence encoding a recombinase encodes SEQ ID NO: 38 or functional variant of SEQ ID NO:38 comprising about 70%, 75%, 80%, 85%, 90%, 91%, 9% 2, 93%, 94%, 95%, 96%, 97%, 98%, or 99% sequence identity to SEQ ID NO:38. In some embodiments, the one or more sequence encoding a recombinase encodes SEQ ID NO: 39 or functional variant of SEQ ID NO:39 comprising about 70%, 75%, 80%, 85%, 90%, 91%, 9% 2, 93%, 94%, 95%, 96%, 97%, 98%, or 99% sequence identity to SEQ ID NO:39.

The disclosure also relates to a nucleic acid sequence or a cell comprising a transduction targeting element comprising a selection marker, or selection nucleic acid sequence. In some embodiments, the selection marker encodes an amino acid comprising SEQ ID NO: 40 or functional variant of SEQ ID NO:40 comprising about 70%, 75%, 80%, 85%, 90%, 91%, 9% 2, 93%, 94%, 95%, 96%, 97%, 98%, or 99% sequence identity to SEQ ID NO:40. In some embodiments, the selection marker encodes comprises a nucleic acid sequence of SEQ ID NO: 41 or functional variant of SEQ ID NO:41 comprising about 70%, 75%, 80%, 85%, 90%, 91%, 9% 2, 93%, 94%, 95%, 96%, 97%, 98%, or 99% sequence identity to SEQ ID NO:41. In some embodiments, the selection marker encodes an amino acid comprising SEQ ID NO: 42 or functional variant of SEQ ID NO:42 comprising about 70%, 75%, 80%, 85%, 90%, 91%, 9% 2, 93%, 94%, 95%, 96%, 97%, 98%, or 99% sequence identity to SEQ ID NO:42. In some embodiments, the selection marker encodes an amino acid comprising SEQ ID NO: 43 or functional variant of SEQ ID NO:43 comprising about 70%, 75%, 80%, 85%, 90%, 91%, 9% 2, 93%, 94%, 95%, 96%, 97%, 98%, or 99% sequence identity to SEQ ID NO:43. In some embodiments, the selection marker encodes an amino acid comprising SEQ ID NO: 44 or functional variant of SEQ ID NO:44 comprising about 70%, 75%, 80%, 85%, 90%, 91%, 9% 2, 93%, 94%, 95%, 96%, 97%, 98%, or 99% sequence identity to SEQ ID NO:44. In some embodiments, the selection marker encodes an amino acid comprising SEQ ID NO: 46 or functional variant of SEQ ID NO:46 comprising about 70%, 75%, 80%, 85%, 90%, 91%, 9% 2, 93%, 94%, 95%, 96%, 97%, 98%, or 99% sequence identity to SEQ ID NO:46. In some embodiments, the selection marker encodes an amino acid comprising SEQ ID NO: 47 or functional variant of SEQ ID NO:47 comprising about 70%, 75%, 80%, 85%, 90%, 91%, 9% 2, 93%, 94%, 95%, 96%, 97%, 98%, or 99% sequence identity to SEQ ID NO:47. In some embodiments, the nucleic acid sequence or the transduction targeting sequence comprises SEQ ID NO: 45 or functional variant of SEQ ID NO:45 comprising about 70%, 75%, 80%, 85%, 90%, 91%, 9% 2, 93%, 94%, 95%, 96%, 97%, 98%, or 99% sequence identity to SEQ ID NO:45.

In some embodiments, the nucleic acid sequence of the disclosure or the cell or cells of disclosure comprise SEQ ID NO:1, SEQ ID NO:10 in two positions, a first promoter and a second promoter individually selectable and chosen from any of SEQ ID NO:3 through SEQ ID NO:9; a nucleic acid sequence encoding a recombinase that is SEQ ID NO:35; an antibiotic resistance sequence comprising SEQ ID NO:41; and a negative selection element comprising SEQ ID NO:45; or any functional variants of any of the foregoing.

The disclosure relates to viral vector comprising one or two nucleic acid molecules as payload or a cell comprising a exogenous nucleic acid sequence comprising a transduction targeting element, optionally stably integrated within its endogenous DNA. In some embodiments, where the viral vector comprises a first and second nucleic acid molecule, the nucleic acid molecules include transduction targeting sequence that is identical or substantially identical to each other. Some embodiments comprise compositions of cells comprises a stably integrated transduction targeting element. In some embodiments, the transduction targeting element comprises one or a combination of: an expressible probe, a DNA barcode, a nucleic acid sequence encoding an immunogen, or any nucleic acid sequence encoding a target protein of choice. These cells can be exposed to a plasmid comprising viral packaging sequences of and replication-deficient virus machinery that allows for transfection of the plasmid DNA into the cell. Upon entry into the cell, cell machinery can encoding the viral packaging machinery for a time period sufficient to assemble replication-deficient viruses. In some embodiments, the viruses are lentiviruses or AAVs. Due to the presence of the transduction targeting sequence in the cells and the viral machinery, viral vectors can be expressed by the cells carrying the transduction targeting element comprising one or a combination of: an expressible probe, a DNA barcode, a nucleic acid sequence encoding an immunogen, or any nucleic acid sequence encoding a target protein of choice, and each virus should be genetically identical or substantially identical and or carry homologous protein on its surface. After isolating the viruses, each cell can independently create a genetically identical or substantially identical virus corresponding to each unique transduction targeting element. After isolation of multiple viruses from multiple different cell lines, each carrying a unique transduction targeting element, compositions of the disclosure include compositions comprising a heterologous population of viruses, each virus comprising a protein and/or genetic material corresponding to an independently selectable transduction targeting element in the cell from which the viral vector is derived.

Viral packaging and production of lentiviruses and AAV particles are generally known. Non-limiting examples of preparing recombinant DNA comprising packaging elements for generation of lentiviral particles are disclosed in WO/1999/031251, which is incorporated by reference in its entirety. Briefly, he method of the disclosure provides, in some embodiments, three vectors which provide all of the functions required for packaging of recombinant virions, such as, gag, pol, env, tat and rev, as discussed above. As noted herein, tat may be deleted functionally for unexpected benefits. There is no limitation on the number of vectors which are utilized so long as the vectors are used to transform and to produce the packaging cell line to yield recombinant lentivirus.

The vectors are introduced via transfection or infection into the packaging cell line. The packaging cell line produces viral particles that contain the vector genome. Methods for transfection or infection are well known by those of skill in the art. After co-transfection of the packaging vectors and the transfer vector to the packaging cell line, the recombinant virus is recovered from the culture media and titered by standard methods used by those of skill in the art.

Thus, the packaging constructs can be introduced into human cell lines by calcium phosphate transfection, lipofection or electroporation, generally together with a dominant selectable marker, such as neo, DHFR, Gin synthetase or ADA, followed by selection in the presence of the appropriate drug and isolation of clones. The selectable marker gene can be linked physically to the packaging genes in the construct.

Stable cell lines wherein the packaging functions are configured to be expressed by a suitable packaging cell are known. For example, see U.S. Pat. No. 5,686,279; and Ory et al., Proc. Natl. Acad. Sci. (1996) 93:11400-11406, which describe packaging cells. Zufferey et al., supra, teach a lentiviral packaging plasmid wherein sequences 3′ of pol including the HIV-1 env gene are deleted. The construct contains tat and rev sequences and the 3′ LTR is replaced with poly A sequences. The 5′ LTR and psi sequences are replaced by another promoter, such as one which is inducible. For example, a CMV promoter or derivative thereof can be used.

Packaging vectors of interest can contain additional changes to the packaging functions to enhance lentiviral protein expression and to enhance safety. For example, all of the HIV sequences upstream of gag can be removed. Also, sequences downstream of env can be removed. Moreover, steps can be taken to modify the vector to enhance the splicing and translation of the RNA. To provide a vector with an even more remote possibility of generating replication competent lentivirus, the instant disclosure provides for lentivirus packaging plasmids wherein tat sequences, a regulating protein which promotes viral expression through a transcriptional mechanism, are deleted functionally. Thus, the tat gene can be deleted, in part or in whole, or various point mutations or other mutations can be made to the tat sequence to render the gene non-functional. An artisan can practice known techniques to render the tat gene non-functional.

The techniques used to construct vectors, and to transfect and to infect cells, are practiced widely in the art. Practitioners are familiar with the standard resource materials which describe specific conditions and procedures. However, for convenience, the following paragraphs may serve as a guideline.

Construction of the vectors of the disclosure employs standard ligation and restriction techniques which are well understood in the art (see Maniatis et al., in Molecular Cloning: A Laboratory Manual, Cold Spring Harbor Laboratory, N.Y., 1982). Isolated plasmids, DNA sequences or synthesized oligonucleotides are cleaved, tailored and religated in the form desired. Site-specific DNA cleavage is performed by treating with the suitable restriction enzyme (or enzymes) under conditions which are understood in the art, and the particulars of which are specified by the manufacturer of the commercially available restriction enzymes, see, e.g. New England Biolabs, Product Catalog. In general, about 1 μg of plasmid or DNA sequences is cleaved by one unit of enzyme in about 20 μl of buffer solution. Typically, an excess of restriction enzyme is used to ensure complete digestion of the DNA substrate. Incubation times of about one hour to two hours at about 37° C. are workable, although variations can be tolerated. After each incubation, protein is removed by extraction with phenol/chloroform, which may be followed by ether extraction, and the nucleic acid recovered from aqueous fractions by precipitation with ethanol. If desired, size separation of the cleaved fragments may be performed by polyacrylamide gel or agarose gel electrophoresis using standard techniques. A general description of size separations is found in Methods of Enzymology 65:499-560 (1980).

Restriction cleaved fragments may be blunt ended by treating with the large fragment of E. coli DNA polymerase I (Klenow) in the presence of the four deoxynucleotide triphosphates (dNTP's) using incubation times of about 15 to 25 minutes at 20° C. in 50 mM Tris (pH 7.6)” 50 mM NaCl, 6 mM MgCl2, 6 mM DTT and 5-10 μM dNTP's. The Klenow fragment fills in at 5′ sticky ends but chews back protruding 3′ single strands, even though the four dNTP's are present. If desired, selective repair can be performed by supplying only one of the dNTP's, or with selected dNTP's, within the limitations dictated by the nature of the sticky ends. After treatment with Klenow, the mixture is extracted with phenol/chloroform and ethanol precipitated. Treatment under appropriate conditions with SI nuclease or Bal-31 results in hydrolysis of any single-stranded portion.

Ligations can be performed in 15-50 μl volumes under the following standard conditions and temperatures: 20 mM Tris-Cl pH 7.5, 10 M MgCl2, 10 mM DTT, 33 mg/ml BSA, 10 mM-50 mM NaCl and either 40 μM ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 33-100 μg/ml total DNA concentrations (5-100 mM total end concentration). Intermolecular blunt end ligations (usually employing a 10-30 fold molar excess of linkers) are performed at 1 μM total ends concentration.

Lentiviral packaging vector is made to contain a promoter and other optional or requisite regulatory sequences as determined by the artisan, gag, pol, rev, env or a combination thereof, and with specific functional or actual excision of tat, and optionally other lentiviral accessory genes. Lentiviral transfer vectors (Naldini et al., supra; Proc. Natl. Acad. Sci. (1996) 93:11382-11388) have been used to infect human cells growth-arrested in vitro and to transduce neurons after direct injection into the brain of adult rats. The vector was efficient at transferring marker genes in vivo into the neurons and long term expression in the absence of detectable pathology was achieved. Animals analyzed ten months after a single injection of the vector, the longest time tested so far, showed no decrease in the average level of transgene expression and no sign of tissue pathology or immune reaction. (Blomer et al., J. Virol. (1997) 71:6641-6649). An improved version of the lentiviral vector in which the HIV virulence genes env, vif, vpr, vpu and nef were deleted without compromising the ability of the vector to transduce non-dividing cells have been developed. The multiply attenuated version represents a substantial improvement in the biosafety of the vector (Zufferey et al., supra). In transduced cells, the integrated lentiviral vector generally has an LTR at each termini. The 5′ LTR may cause accumulation of “viral” transcripts that may be the substrate of recombination, in particular in HIV-infected cells. The 3′ LTR may promote downstream transcription with the consequent risk of activating a cellular protooncogene. The U3 sequences comprise the majority of the HIV LTR. The U3 region contains the enhancer and promoter elements that modulate basal and induced expression of the HIV genome in infected cells and in response to cell activation. Several of the promoter elements are essential for viral replication. Some of the enhancer elements are highly conserved among viral isolates and have been implicated as critical virulence factors in viral pathogenesis. The enhancer elements may act to influence replication rates in the different cellular target of the virus (Marthas et al. J. Virol. (1993) 67:6047-6055). As viral transcription starts at the 3′ end of the U3 region of the 5′ LTR, those sequences are not part of the viral mRNA and a copy thereof f om the 3′LTR acts as template for the generation of both LTR's in the integrated provirus. If the 3′ copy of the U3 region is altered in a retroviral vector construct, the vector RNA still is produced from the intact 5′ LTR in producer cells, but cannot be regenerated in target cells.

Transduction of such a vector results in the inactivation of both LTR's in the progeny virus. Thus, the retrovirus is self-inactivating (SIN) and those vectors are known as Sin transfer vectors.

There are, however, limits to the extent of the deletion at the 3′ LTR. First, the 5′ end of the U3 region serves another essential function in vector transfer, being required for integration (terminal dinucleotide+att sequence). Thus, the terminal dinucleotide and the att sequence may represent the 5′ boundary of the U3 sequences which can be deleted. In addition, some loosely defined regions may influence the activity of the downstream polyadenylation site in the R region. Excessive deletion of U3 sequence from the 3′ LTR may decrease polyadenylation of vector transcripts with adverse consequences both on the titer of the vector in producer cells and the transgene expression in target cells. On the other hand, limited deletions may not abrogate the transcriptional activity of the LTR in transduced cells.

Packaging of AAV vectors is also generally known. For packaging and production of adeno-associated viruses, serotype 2 (AAV2), un-engineered and engineered HEK 293F were transfected with a helper plasmid, a virus plasmin containing AAV Rep and Cap proteins, and a transfer plasmid expressing NeonGreen fluorescent protein under the control of a constitutive cytomegalovirus (CMV) early promoter, flanked by AAV2 inverted terminal repeats (ITRs), and an additional plasmid expressing microRNA, mi342 under the control of a ubiquitous CMV promoter. Transfections were performed using linear polyethyleneimine (PEI). Post-transfection, crude virus was extracted from cell lysates and virus was recovered by centrifugation. Quantitative PCR was used to measure viral copy number produced by un-engineered and engineered HEK 293 cell lines. Disclosure including the packaging proteins for AAV as well as methods of preparing an transfecting helper plasmids are disclosed in WO/2023/239928, which is incorporated by reference in its entirety. An AAV Rep protein sequence is:

(SEQ ID NO: 48)
MPGFYEIVIKVPSDLDEHLPGISDSFVNWVAEKEWELPPDSDMDLNLI
EQAPLTVAEKLQRDFLTEWRRVSKAPEALFFVQFEKGESYFHMHVLVE
TTGVKSMVLGRFLSQIREKLIQRIYRGIEPTLPNWFAVTKTRNGAGGG
NKVVDECYIPNYLLPKTQPELQWAWTNMEQYLSACLNLAERKRLVAQH
LTHVSQTQEQNKENQNPNSDAPVIRSKTSARYMELVGWLVDKGITSEK
QWIQEDQASYISFNAASNSRSQIKAALDNAGKIMSLTKTAPDYLVGQQ
PVEDISSNRIYKILELNGYDPQYAASVFLGWATKKFGKRNTIWLFG′P
ATTGKTNIAEAIAHTVPFYGCVNWTNENFPFNDCVDKMVIWWEEGKMT
AKVVESAKAILGGSKVRVDQKCKSSAQIDPTPVIVTSNTNMCAVIDGN
STTFEHQQPLQDRMFKFELTRRLDHDFGKVTKQEVKDFFRWAKDHVVE
VEHEFYVKKGGAKKRPAPSDADISEPKRARESVAQPSTSDAEASINYA
DRYQNKCSRHVGMNLMLFPCRQCERMNQNSNICFTHGQKDCLECFPVS
ESQPVSVVKKAYQKLCYIHHIMGKVPDACTACDLVNVDLDDCISEQ
An AAV Cap sequence is
(SEQ ID NO: 49)
MAADGYLPDWLEDTLSEGIRQWWKLKPGPPPPKPAERHKDDSRGLVLP
GYKYLGPFNGLDKGEPVNEADAAALEHDKAYDRQLDSGDNPYLKYNHA
DAEFQERLKEDTSFGGNLGRAVFQAKKRVLEPLGLVEEPVKTAPGKKR
PVEHSPVEPDSSSGTGKAGQQPARKRLNFGQTGDADSVPDPQPLGQPP
AAPSGLGSTTMATGSGAPVADNNEGADGVGNSSGNWHCDSQWLGDRVI
TTSTRTWALPTYNNHLYKQISSQSGASNDNHYFGYSTPWGYFDFNRFH
CHFSPRDWQRLINNNWGFRPKRLNFKLFNIQVKEVTQNDGTTTIANNL
TSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDR
LMNPLIDQYLYYLNKTQTNSGTLQQSRLLFSQAGPTNMSLQAKNWLPG
PCYRQQRLSKQANDNNNSNFPWTAATKYHLNGRDSLVNPGPAMASHKD
DEEKFFPMHGTLIFGKQGTNANDADLENVMITDEEEIRTTNPVATEQY
GTVSNNLQNSNTGPTTGTVNHQGALPGMVWQDRDVYLQGPIWAKIPHT
DGHFHPSPLMGGFGLKHPPPQIMIKNTPVPANPPTNFSSAKFASFITQ
YSTGQVSVEIEWELQKENSKRWNPEIQYTSNYNKSVNVDFTVDINGVY
SEPRPIGTRYLTRNL

Nucleic acids comprising a nucleic acid sequence encoding REP and CAP can be transfected into cells disclosed in the present application to produce AAV particles comprising the payload with a positive and negative strand of viral nucleic acid sequences derived from the disclosed cells.

The disclosure also relates to compositions comprising a guide sequence and/or a crRNA-tracrRNA duplex, wherein the guide sequence or crRNA-tracrRNA comprises a DNA-binding domain (the sequence complementary to a target sequence of choice) comprising at least one unmodified nucleotide. This is particularly useful in the preparation of cells comprising the disclosed transduction targeting element.

In some embodiments, the disclosure relates to compositions comprising a disclosed cell and one or a plurality of guide sequences and/or a crRNA-tracrRNA duplexes, wherein the guide sequence and/or the crRNA-tracrRNA duplex comprises a DNA-binding domain comprising at least one nucleotide comprising an unmodified hydroxyl or hydrogen substituent at its 2′ carbon in its sugar moiety. In some embodiments, the disclosure relates to compositions comprising a guide sequence and/or a crRNA-tracrRNA duplex, wherein the a guide sequence and/or a crRNA-tracrRNA duplex comprises a DNA-binding domain comprising at least one nucleotide comprising an unmodified hydroxyl group at its 2′ carbon in its sugar moiety. In some embodiments, the disclosure relates to compositions comprising a guide sequence and/or a crRNA-tracrRNA duplex, wherein a guide sequence and/or a crRNA-tracrRNA duplex comprises a DNA-binding domain comprising one or a combination of unmodified hydroxyl group at its 2′ carbon in its sugar moiety. In some embodiments, the disclosure relates to compositions comprising a guide sequence and/or a crRNA-tracrRNA duplex, wherein the a guide sequence and/or a crRNA-tracrRNA duplex comprises a DNA-binding domain comprising 1, 2, 3, 4, 5, 6, 7, 8, 9, 10, 11, 12, 13, 14, 15, 16, 17, 18, 19, 20, 21, 22, 23, 24, 25, 26, or 27 of the unmodified hydroxyl groups at the 2′ carbon in its sugar moiety.

In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 0% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 10% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 20% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 30% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 40% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 50% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 60% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 70% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 80% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 90% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a transcription terminator domain comprising from about 95% to about 100% modified nucleotides.

In some embodiments, the disclosure relates to compositions comprising a guide sequence and/or a crRNA-tracrRNA duplex, wherein the guide sequence and/or a crRNA-tracrRNA duplex comprises a nucleotide binding domain (such as a DNA-binding domain) comprising at least one modified nucleotide. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising at least one modified nucleotide at its 2′ carbon. In some embodiments, the disclosure relates to compositions comprising a guide sequence and/or a crRNA-tracrRNA duplex, wherein the guide a sequence and/or a crRNA-tracrRNA duplex comprises a nucleotide binding domain comprising from about 1% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 10% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 20% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 30% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 40% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 50% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 60% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 70% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 80% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 90% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 95% to about 100% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 35% to about 75% modified nucleotides. In some embodiments, the disclosure relates to compositions comprising a guide sequence and/or a crRNA-tracrRNA duplex, wherein the guide sequence and/or a crRNA-tracrRNA duplex comprises a nucleotide binding domain comprising from about 40% to about 60% modified nucleotides.

In some embodiments, the disclosure relates to compositions comprising a guide sequence comprising, consisting essentially of, or consisting of a sequence that 70%, 75%, 80%, 85%, 90%, 91%, 92%, 93%, 94%, 95%, 96%, 97%, 98%, 99% to the RNA sequence: UUAUCAACUUGAAAAAGUGGCACCGAGUCGGUGCUUUUUUU (SEQ ID NO: 50). In some embodiments, the disclosure relates to compositions comprising a guide sequence comprising, consisting essentially of, or consisting of a sequence that 70%, 75%, 80%, 85%, 90%, 91%, 92%, 93%, 94%, 95%, 96%, 97%, 98%, 99% to SEQ ID NO: 50 or a functional variant thereof, wherein the guide sequence comprises at one modified nucleotide. In some embodiments, the disclosure relates to compositions comprising a guide sequence comprising, consisting essentially of, or consisting of a sequence that 70%, 75%, 80%, 85%, 90%, 91%, 92%, 93%, 94%, 95%, 96%, 97%, 98%, 99% to SEQ ID NO: 50, wherein the guide sequence comprises at least one modified nucleotide at its 2′ carbon. In some embodiments, the disclosure relates to compositions comprising a guide sequence comprising, consisting essentially of, or consisting of a sequence that 70%, 75%, 80%, 85%, 90%, 91%, 92%, 93%, 94%, 95%, 96%, 97%, 98%, 99% to SEQ ID NO: 50. In some embodiments, the guide sequence comprises a transduction targeting element disclosed herein or a nucleic acid sequence disclosed herein.

Compositions of the disclosure may also include Cas proteins and cells of the disclosure. Another aspect of the disclosure relates to a CRISPR system comprising a modified CRISPR enzyme (or “Cas protein”) or a nucleotide sequence encoding one or more Cas proteins. Any protein capable of enzymatic activity in cooperation with a guide sequence is a Cas protein. In some embodiments, the disclosure relates to a system comprises a vector comprising a regulatory element operably linked to an enzyme-coding sequence encoding a CRISPR enzyme, such as a Cas protein from the Cas family of enzymes. In some embodiments, the disclosure relates to a system, composition, comprising any one or plurality of Cas proteins either individually or in combination with one or a plurality of guide sequences, and one or a plurality of cells comprising the transduction targeting element. Compositions of one or a plurality of Cas proteins may be administered to or exposed to a cell with any of the disclosed guide sequences sequentially or contemporaneously. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, type V CRISPR-Cas systems, variants and fragments thereof, or modified versions thereof having at least 70% homology to the sequences of Table Y, wherein are incorporated by reference in their entireties. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme or Cas protein that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In some embodiments, a Cas9 nickase may be used in combination with guide sequenc(es), e.g., two guide sequences, which target respectively sense and antisense strands of the DNA target. This combination allows both strands to be nicked and used to induce NHEJ.

The disclosure also relates to a system comprising a cell, a guide sequence comprising one or a plurality of transduction targeting elements, and a Cas protein. Methods of altering the genomic DNA of a cell comprise: exposing the cell to a guide sequence comprising one or a plurality of signal transduction sequences, and a Cas protein; and allowing the Cas protein to modify the genomic DNA of the cell to stably integrate the transduction targeting sequence.

According to one aspect, the nucleic acid disclosed herein comprises from about 10 to about 250 nucleotides. According to one aspect, the nucleic acid disclosed herein comprises from about 20 to about 100 nucleotides.

The disclosure relates to a composition comprising a cell with any one or combination of nucleic acid sequences disclosed herein. In some embodiments, the cell is a plant, insect or mammalian cell. In some embodiments, the cell is a eukaryotic cell or a prokaryotic cell, the cell may be isolated from the body, a component of a culture system, or part of an organism. Conditions which enable formation of the virus vector of the disclosure are well known in the art. These conditions may vary depending upon the properties of the producer cell and the enveloped virus used. A number of references exist which describe conditions which are useful for culturing particular viruses (Fields Virology, 3rd ed., Fields et al., eds., Lippincott-Raven Publishers, Philadelphia, Pa.). Particular non-limiting examples are provided herein of conditions which are useful to enable formation of the enveloped virus vector of the disclosure. In some embodiments, the cell is a mammalian cell. In some embodiments, the cell is a 293 cell, CHO cell, 293T cell, or one of the following cells characterized by the accompanying webpage, which is incorporated by reference in its entirety:

NIH/3T3 https://www.atcc.org/products/crl-1658 K-562 https://www.atcc.org/products/ccl-243 B16-F10 https://www.atcc.org/products/crl-6475 HeLa https://www.atcc.org/products/ccl-2 MDCK https://www.atcc.org/products/ccl-34 BHK-21 https://www.atcc.org/products/ccl-10 CHO-K1 https://www.atcc.org/products/ccl-61 MCF7 https://www.atcc.org/products/htb-22 Vero https://www.atcc.org/products/ccl-81

Conditions which enable formation of the virus of the disclosure include conditions which enable expression of the competent portion of the genome of the enveloped virus, conditions under which viral proteins from its packaging machinery are present in the membrane of the producer cells disclosed herein, and conditions which enable the formation of viral particles from the components of a producer cell which has been provided with the competent portion of the genome, as well as efficiency of packaging DNA from the cell copied and flanked by the viral tandem repeats. Further details regarding processes by which viral particles are formed following provision to a cell of a competent portion of the genome of an enveloped virus have been described in the art, for instance by Wiley (1985, in Virology, Fields et al., ed., Raven Press, New York, 45-52).

In some embodiments, a system comprising a cell culture unit is utilized to culture and expand a disclosed cell population described herein. In some embodiments, the cell culture unit comprises one or a plurality of cell reactor surfaces housed in at least a first compartment, the one or plurality of cell reactor surfaces in fluid connection with a first and second media line, the first media line in fluid communication with a first media inlet, the second media line in fluid communication to a first media outlet. In some embodiments, the one or plurality of cell reactor surfaces are configured in a cylindrical form with a hollow volume fixed within a cylindrical first compartment; wherein the first media line and the second media line are positioned on opposite faces of the cylindrical first compartment. The first media line can be attached to a first sealable aperture configured for sterile attachment of a cell culture media source. In some embodiments, the system further comprises a pump and a fluid regulator in operable contact with the first media line, wherein the pump is capable of generating pressure in the first media line and wherein the fluid regulator is capable of regulating the speed of fluid from the pump through the first compartment and into the second media line.

The one or plurality of cell reactor surfaces can have a surface area from about 0.5 m² to about 100.0 m², including any value therein, such as about 3 m², about 4 m², about 5 m², about 6 m², about 7 m², about 8 m², about 9 m², about 10 m², about 11 m², about 12 m², about 13 m², about 14 m², about 15 m², about 16 m², about 17 m², about 18 m², about 19 m², about 20 m², about 21 m², about 22 m², about 23 m², about 24 m², about 25 m², about 26 m², about 27 m², about 28 m², about 29 m², about 30 m², about 31 m², about 32 m², about 33 m², about 34 m², about 35 m², about 36 m², about 37 m², about 38 m², about 39 m², about 40 m², about 41 m², about 42 m², about 43 m², about 44 m², about 45 m², about 46 m², about 47 m², about 48 m², about 49 m², about 50 m², about 51 m², about 52 m², about 53 m², about 54 m², about 55 m², about 56 m², about 57 m², about 58 m², about 59 m², about 60 m², about 61 m², about 62 m², about 63 m², about 64 m², about 65 m², about 66 m², about 67 m², about 68 m², about 69 m², about 70 m², about 71 m², about 72 m², about 73 m², about 74 m², about 75 m², about 76 m², about 77 m², about 78 m², about 79 m², about 80 m², about 81 m², about 82 m², about 83 m², about 84 m², about 85 m², about 86 m², about 87 m², about 88 m², about 89 m², about 90 m², about 91 m², about 92 m², about 93 m², about 94 m², about 95 m², about 96 m², about 97 m², about 98 m², or about 99 m², or about 100 m², or about 105 m².

Some embodiments of the system further comprise a gas transfer module in operable connection to the one or plurality of cell reactor surfaces. In some embodiments, the gas module comprises a gas pump and a gas regulator connected to the first compartment by a first gas line. In such embodiments, the first compartment comprises at least one gas outlet. The gas pump is capable of generating air pressure from the pump to the first compartment through the first gas line. The gas outlet can be one or more vents or the gas outlet can be configured for sterile connection to one or more vents. The gas regulator is capable of regulating the speed of gas from the pump through the first compartment.

Some embodiments further comprise a first gas inlet in operable connection to the gas transfer module. In some embodiments, the first gas inlet is attached to a second sealable aperture configured for sterile attachment of a gas source. The gas source can be any known gas storage and/or delivery system, such as for example a container or a tank.

The system can further comprise an apheresis unit in fluid communication with the cell culture unit. Suitable apheresis units include the Spectra Optia Apheresis System (TerumoBCT).

Additionally, in some embodiments, the system further comprises a harvesting compartment in fluid communication with the cell culture unit. Suitable harvesting compartments are discussed elsewhere herein.

A cell culture system as described herein can be used to expand cells from a subject through culturing one or a plurality of cells in the system and allowing the cells to grow in the first compartment for a time period sufficient to proliferate. The cells can be introduced into the system through the system's first compartment. In some embodiments, the cells are 293T-cells, NIH3T3 cells, HeLa cells, or any of the foregoing cells disclosed herein.

Some embodiments of the system and methods described herein include at least two components: (1) the RNAs or DNA/RNA hybrid (guide nucleic acid, a crRNA, tracrRNA, and/or a single cr/tracrRNA hybrid) targeted to a particular sequence in a cell (e.g., either genomic DNA, or in an extrachromosomal plasmid, such as a reporter); and (2) a Cas protein disclosed herein. In some cases, a system also can include a nucleic acid containing a donor sequence targeted to a sequence in the cell. The donor sequence and the guide sequence may be on one or a plurality of nucleic acid molecules. The Cas protein disclosed herein can create targeted DNA double-strand breaks at the desired locus (or loci), and the host cell can repair the double-strand break using the provide donor DNA sequence, thereby incorporating the modification stably into the host genome.

The construct(s) containing the guide RNA or RNA/DNA hybrid molecules, crRNA, tracrRNA, cr/tracrRNA hybrid, Cas protein disclosed herein coding sequence, and, where applicable, donor sequence, can be delivered to a cell using, for example, biolistic bombardment, electrostatic potential or through transformation permeability reagents (reagents known to increase the permeability of the cell wall or cell membrane). After an organism or cell is infected, administered or transfected with a nucleic sequence disclosed herein with a landing pad, a sequence encoding a Cas protein disclosed herein or a functional fragment thereof, a crRNA, a trRNA, a crRNA and a tracrRNA, a cr/tracrRNA hybrid, and/or a synthetic guide nucleic acid (and, in some cases, a donor sequence), any suitable method can be used to determine whether targeted mutagenesis has occurred at a target site within the genome of the cell. In some embodiments, a phenotypic change can indicate that a donor sequence has been integrated into the target site. Such is the case for cells encoding a reporter gene, for example. PCR-based methods also can be used to ascertain whether a genomic target site contains targeted mutations or donor sequence, and/or whether precise recombination has occurred at the 5′ and 3′ ends of the donor.

Compositions of the disclosure also relate to a library of cells, wherein each cell element of the library comprises a transduction targeting element unique to the cell element. As an example, a plurality of nucleic acid molecules each carrying a different target protein or payload encoding a target protein or comprising a nucleic acid sequence can be transfected into the cells such that the cells are genetically altered to produce the payload or target protein. Expression of the protein can be incorporated into viral particles by transfection of nucleic acid sequences that encode envelope and capsid proteins of viruses, whereupon exposure to the tandem repeats in the transduction targeting element, the payload is packaged in the viral particles and exocytosed from the producer cell. In another variation of this method, a library comprising a plurality of nucleic acid-containing vectors is provided, each of which comprises a nucleic acid which corresponds to at least a portion of a viral nucleic acid, wherein when a vector selected from the library is provided to a cell, any protein encoded by the nucleic acid of that vector is capable of expression in the cell. Methods of making such vectors are well known to one skilled in the art of molecular biology and are described in such references as Sambrook et al. (1989, Molecular Cloning: A Laboratory Manual, Cold Spring Harbor Laboratory, New York). An individual vector is provided to a control cell and is expressed therein to generate a test cell. The resulting viral particle comprising the payload of the vector may subsequently be isolated, cloned, interrogated and characterized using techniques well known in the art. Embodiments provide viral particles that comprise proteins from flavivirus, such as but not limited to, Dengue, Zika, Yellow Fever, Japanese encephalitis, and West Nile, either wild-type or a wild-type sequence having one or more mutations (a variant), such as those described herein, that result in decreased or no infectivity while allowing for the virus to be expressed and bud, thereby allowing for a virion to deliver a payload to a target cell. In some embodiments, the mutation leaves the antigenic reactivity of the strain the same (ie reactivity with MAbs that are sensitive to its conformation). In some embodiments, the mutation decreases or inhibits infectivity, does not effect, or improves tropism to a target cell, increases budding and/or increases expression on the lipid bilayer on a disclosed cell or cell line. The viral particles (including chimeras) described herein (eg, those that include one or more of the mutations described herein) can be made using standard methods in the art. For example, an RNA molecule corresponding to the genome of a virus can be introduced into primary cells, chick embryos, or diploid cell lines, from which (or the supernatants of which) progeny viruses can then be purified. Another method that can be used to produce the viruses employs heteroploid cells, such as Vero cells (Yasumura et al., Nihon Rinsho 21, 1201-1215, 1963). In this method, a nucleic acid molecule (eg, an RNA molecule) corresponding to the genome of a virus is introduced into the heteroploid cells, virus is harvested from the medium in which the cells have been cultured, harvested virus is treated with a nuclease (eg, an endonuclease that degrades both DNA and RNA, such as Benzonase™. U.S. Pat. No. 5,173,418 the nuclease-treated virus is concentrated (eg, by use of ultrafiltration using a filter having a molecular weight cut-off of, eg, 500 kDa), and the concentrated virus is formulated for the purposes of vaccination. Methods of making viral particle comprising flaviviral proteins are disclosed in WO/2016/130786, which is incorporated herein by reference in its entirety.

### Methods

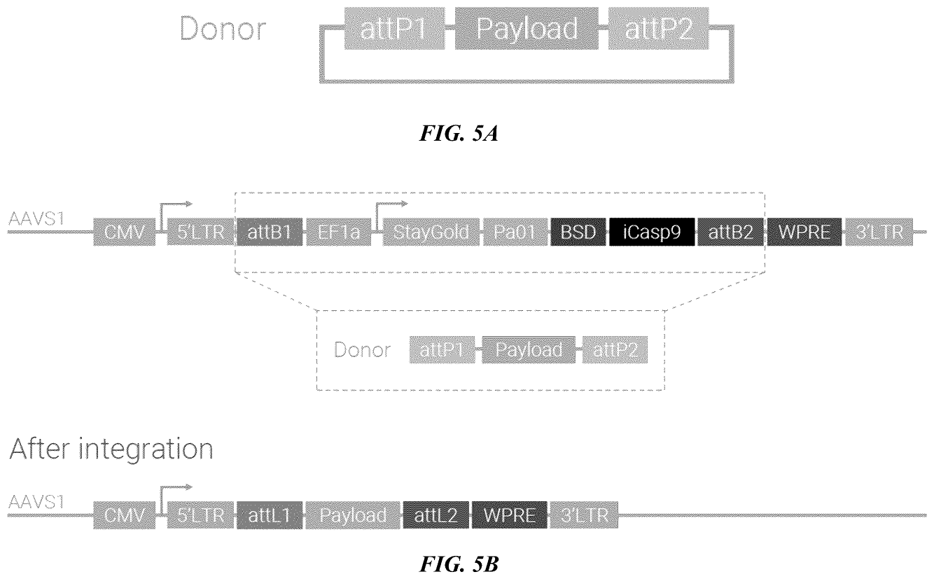

In some embodiments, the disclosure provides a method for producing a library of cell clones that are each clonal and endogenously homogeneous for a single integrated viral payload, such as a lentiviral payload. As used herein the term “homogeneous for a single integrated viral payload” means that the clonal population of cells from each successful transduction event contains a single unique heterologous DNA element such as a transduction targeting element. The method according to this aspect comprises (a) providing one or a plurality of landing pad cells according to the first aspect of the disclosure, (b) transfecting the landing pad cells with a library of plasmids comprising, from 5′ to 3′, an attP1 element, a payload element and an attP2 element, wherein the attP1 and attP2 elements are recognized by the serine recombinase and the payload element comprises a target DNA and a gene encoding a targeting protein or a functional fragment thereof (FIG. 5A), (c) allowing the plasmid and the landing pad to undergo site-specific recombination, (d) inducing expression of the cell death gene to select for cells in which the plasmid and the landing pad have undergone site-specific recombination, and (e) culturing the surviving cells to produce lentivirus particles expressing the target DNA. As shown in FIG. 5, site-specific recombination between the attB and attP sites results in integration of the viral payload into the chromosome and removes the cell death gene from the chromosome (FIG. 5B). Induction of the cell death gene allows selection of cells that have undergone site-specific recombination between the landing pad and the donor plasmid.

According to one aspect, the disclosure relates to a method of altering genomic DNA of a eukaryotic cell one or a plurality of sites comprising: transfecting the eukaryotic cell comprising any transduction targeting element disclosed herein with one or a plurality of nucleic acids complementary to different sites on genomic DNA of the eukaryotic cell, transfecting the eukaryotic cell with a nucleic acid encoding an enzyme that interacts with the nucleic acid complementary to different sites on genomic DNA of the eukaryotic cell, such that the enzyme cleaves the genomic DNA in a site-specific manner, wherein the cell expresses the enzyme, the nucleic acids complementary to different sites on genomic DNA of the eukaryotic cell bind to complementary genomic DNA and the enzyme cleaves the genomic DNA in a site-specific manner. According to one aspect, the enzyme is Cas9. According to one aspect, the eukaryotic cell is a yeast cell, a plant cell or a mammalian cell. In some embodiments, the cells are 293 or 293T cells.

Methods of performing a recombination event in a cell are generally known. But, briefly, PhiC31 recombinase is a DNA recombinase derived from Streptomyces phage cpC31. Other examples of recombinase enzymes are disclosed on Table Z. As a non-limiting example, PhiC31 enzyme can mediate recombination of nucleic acid sequences positioned between two nucleic acid sequences attB and attP. Cre recombinase is also a site-specific recombinase which is used in the present system to subsequently excise the selection system and the plasmid bacterial backbone. Accordingly, the Cre recombinase can be described as “cleaning up” the vector backbone from non-useful sequences once the initial selection has been made. Both PhiC31 recombinase and Cre recombinase are well-known enzymes used in Site Specific Recombination. An example of a matching DNA donor vector includes a first selection marker lacking a promoter encoded in anticlockwise orientation, a matching PhiC31 recombinase recognition sequence (attB1), expression cassette(s) comprising a nucleic acid sequence encoding a protein of interest, a complementing recombinase recognition sequence (loxP) for the Cre recombinase, a fully functional expression cassette for a second selection marker (optional, here exemplified by FC-eGFP) and a plasmid backbone (containing sequences for bacterial propagation etc.).

Co-transfecting the DNA Donor Vector and a vector for expression of PhiC31 into an eukaryotic host cell comprising a pre-defined genomic location of a Landing Pad (LP) sequence, will lead to integration of the donor vector at the LP via PhiC31 mediated recombination of atiP1 and attB2 for a fraction of the transfected cells. Upon integration at the pre-defined genomic location, the promoter-less selection marker will be positioned so that it is activated by the promotor in the pre-defined genomic position. Activity of the first selection marker can then be used to select for cells having undergone integration at the LP (using FACS in the case of RFP). Proper selection should generate a pool of cells where most cells have a single copy integrated at the LP. However, a fraction of the cells is expected to have additional copies integrated via off-target integration mechanisms, such as DNA repair mediated random integration and PhiC31 mediated integration at genomic pseudo-attP sequences. To select against such events and at the same time enable removal of selection marker cassettes and plasmid backbone at the pre-defined genomic location (i.e. a “cleaning up”) a second recombinase mediated step has been designed.

Since the pre-defined genomic location contains a loxP sequence and the DNA Donor Vector also contains a strategically placed loxP sequence, integration events at the pre-defined genomic location will contain both selection markers (as well as other unwanted sequence elements such as the plasmid backbone), flanked by two loxP sequences. In contrast, most off-target events should not lead to loxP flanked selection markers (some random integration events of concatemerized donor vectors could lead to flanked second selection marker genes, but this should be extremely rare). By using a second transfection of a vector encoding the Cre recombinase, the region being flanked by loxP sequences can be excised from the genome of corresponding cells. Cells having a single copy integrated at the pre defined genomic location (lacking off-target integration), as well as having unwanted sequence elements removed, can hence be selected for via the absence of selection marker activity (absence of eGFP activity using FACS). This is also called selection by negative selection.

Non-limiting examples of first DNA enzymes for use in a method as defined herein are DNA recombinases, such as a PhiC31 or Bxb1 recombinase, and as described elsewhere herein. A characterizing feature of a recombinase when used as a first DNA enzyme is that it will introduce, not remove, nucleic acid sequence regions into the pre-defined genomic region.

Non-limiting examples of the second DNA enzyme for use in a method as defined herein are DNA recombinases, such as PhiC31 recombinase, Bxb1 recombinase, Cre recombinase and Dre recombinase, and as described elsewhere herein. A characterizing feature of a recombinase when used as a second DNA enzyme is that it will remove, not introduce, nucleic acid sequence regions from the pre-defined genomic region.

The disclosure relates to a method of producing a virus comprising one or more target proteins, the method comprising steps of culturing any one or plurality of cells disclosed herein, wherein the transduction targeting element comprises an expressible nucleic acid sequence that encodes the target protein; transfecting a plasmid comprising viral packaging and viral machinery proteins, and allowing the cells to produce viruses comprising the target protein. In some embodiments, the transduction targeting element comprises a DNA barcode unique to the cell and transferable to the virus, such that, upon culturing the cells with a plasmid comprising the viral packaging machinery proteins, the cells express a virus comprising the DNA barcode.

The disclosure relates to a method of culturing a cell or cell line comprising:
exposing a cell to a cell culture medium under condition sufficient to grow the cell or cell line. In some embodiments, the cell is exposed to 95% oxygen at 37 degrees Celsius for no less than from about 2 to about 10 days. In some embodiments, the method further comprises:
exposing the cell or cell line to one or a plurality of nucleic acid molecules comprising a payload positioned between a third and a fourth recombinase attachment element; and
allowing a time period sufficient to enable recombination between the nucleic acid molecule comprising the payload and the nucleic acid sequence disclosed herein, such that the payload is exchanged for a region of the transduction targeting element between the first recombinase attachment element and the second recombinase attachment element. In some embodiments, the recombination event is accomplish by inducing expression of the recombinase or integrase expressed by the cell. In some embodiments, the expression is accomplished by a nucleic acid expression of the region of the transduction targeting element comprising the nucleic acid sequence encoding the recombinase. In some embodiments, the method further comprises a step (d) transfecting the cell or cell line with a nucleic acid molecule comprising a nucleic acid sequence encoding a viral packaging protein and/or structural proteins after step (a), (b) and (c). In some embodiments, the method further comprises a step (e) culturing the cell or cell line for a time period sufficient for the cell or cell line to produce a virus comprising the viral packaging protein encapsulating two nucleic acid sequence comprising the payload.

The disclosure also relates to a method of preventing homologous recombination in a cell infected comprising: (a) exposing a cell disclosed herein to a cell culture medium under condition sufficient to grow the cell or cell line. In some embodiments, the method further comprises a step of: exposing the cell or cell line to one or a plurality of nucleic acid molecules comprising a payload positioned between a third and a fourth recombinase attachment element. In some embodiments, the method further comprises a step of: (c) allowing a time period sufficient to enable recombination between the nucleic acid molecule comprising the payload and the nucleic acid sequence disclosed herein, such that the payload is exchanged for a region of the transduction targeting element between the first recombinase attachment element and the second recombinase attachment element. In some embodiments, the method further comprises a step (d) transfecting the cell or cell line with a nucleic acid molecule comprising a nucleic acid sequence encoding viral packaging proteins after step (a), (b) and (c), such that virions are produced by the cell that carry the nucleic acid sequence within the transduction targeting element between the first and second viral tandem repeats and including the payload. In some embodiments, the payload is an antigenic protein, a sequence encoding a chimeric immune receptor, a therapeutic protein, a DNA barcode or a DNA barcode in frame with any one of the foregoing.

The disclosure also relates to a method of modifying the genetic material of a cell comprising: exposing any cell or plurality of cells disclosed herein to a nucleic acid molecule comprising a payload flanked by two recombinase attachment elements capable of aligning to or non covalently binding to a first and a second recombinase attachment element in the genomic DNA of the cell or plurality of cells; allowing the payload to exchange its position from the nucleic acid molecule into the genomic DNA of the cell at the position of the transduction targeting element flanked by the first and second recombinase attachment elements, such that the payload becomes integrated into the genomic DNA of the cell or plurality of cells and the genetic material of the cell becomes modified; wherein, if the composition comprises a plurality of cells, the cells are a clonal population, wherein the cell and the cells in the clonal population of cells comprise an identical or substantially identical transduction targeting element, or landing pad. In some embodiments, the genomic DNA flanked by the first and second recombinase attachment element comprises the nucleic acid sequence encoding the recombinase.

The disclosure also relates to a method of generating a composition comprising a homogenous population of viruses carrying a payload, wherein the viruses comprise an identical or substantially identical viral genome, the method comprising (a) exposing a cell disclosed herein to a cell culture medium under condition sufficient to grow the cell or cell line. In some embodiments, the method further comprises a step of: (a) exposing a cell or cell line disclosed herein to one or a plurality of nucleic acid molecules comprising a payload positioned between a third and a fourth recombinase attachment element. In some embodiments, the method further comprises a step of: (c) allowing a time period sufficient to enable recombination between the nucleic acid molecule comprising the payload and the nucleic acid sequence disclosed herein, such that the payload is exchanged for a region of the transduction targeting element between the first recombinase attachment element and the second recombinase attachment element. In some embodiments, the method further comprises a step (d) transfecting the cell or cell line with a nucleic acid molecule comprising a nucleic acid sequence encoding viral packaging proteins after step (a), (b) and (c), such that virions are produced by the cell that carry the nucleic acid sequence within the transduction targeting element between the first and second viral tandem repeats and including the payload. In some embodiments, the payload is an antigenic protein, a sequence encoding a chimeric immune receptor, a therapeutic protein, a DNA barcode or a DNA barcode in frame with any one of the foregoing.

In any of the above methods, some embodiments comprise a step of characterizing or detecting the presence of the cells or viruses of the disclosure by detecting a probe or expressed to the cell or virus. In some embodiments, the methods comprise a step of exposing the cell or virus to a probe that noncovalently or covalently binds to the cell or virus, and subsequently measuring the presence of the probe. In some embodiments, the step of measuring or detecting the presence of the probe comprises performing one or a plurality of: flow cytometry, chemiluminescence of an enzyme-conjugated antibody, immunosorbent assay, induced fluorescence of an antibody or other protein, performing DNA sequencing or polymerase chain reaction techniques on the payload or barcode component of a payload, and stimulating a color-sensitive dye incorporated into or intercalating with a nucleic acid sequence of the cells such as the transduction targeting element and/or the payload.

Methods of making pseudotypes are generally known The manner of providing the competent portion of the genome of the enveloped virus to the producer cell is also not critical. However, when the competent portion of the genome is expressed in the cell, the formation of at least one enveloped virus-like particle must be enabled. The competent portion of the genome may be provided in the form of, for example, the genome of an enveloped virus, a plasmid, or a non-circularized nucleic acid. The competent portion of the genome maybe, but is not limited to, a single-stranded RNA molecule, a double-stranded RNA molecule, a single-stranded DNA molecule, a double-stranded DNA molecule, or an RNA-DNA hybrid molecule. The enveloped virus may be any enveloped virus, and is preferably a retrovirus. Preferred enveloped viruses are selected from the group consisting of HIV, SIV, RSV, and (ecotropic and amphotropic really refer to the Envelope protein of MLV, not the core) MLV.

Conditions which enable formation of the enveloped virus vector of the disclosure are well known in the art. These conditions may vary depending upon the properties of the producer cell and the enveloped virus used. A number of references exist which describe conditions which are useful for culturing particular enveloped viruses (Fields Virology, 3rd ed., Fields et al., eds., Lippincott-Raven Publishers, Philadelphia, Pa.). Particular non-limiting examples are provided herein of conditions which are useful to enable formation of the enveloped virus vector of the disclosure.

Conditions which enable formation of the enveloped virus vector of the disclosure include conditions which enable expression of the competent portion of the genome of the enveloped virus, conditions under which a cellular virus receptor protein is present in the membrane of the producer cell, and conditions which enable the formation of enveloped virus-like particles from the components of a producer cell which has been provided with the competent portion of the genome. Further details regarding processes by which enveloped viral particles are formed following provision to a cell of a competent portion of the genome of an enveloped virus have been described in the art, for instance by Wiley (1985, in Virology, Fields et al., ed., Raven Press, New York, 45-52).

Another example of making the enveloped virus vector of the disclosure, further comprises providing an additional component to the producer cell, whereby, upon formation of the enveloped virus vector, the enveloped virus vector comprises the additional component. The additional component may be any molecule which can be provided to the cytoplasm or the membrane of the producer cell. By way of example, the additional component may be a nucleic acid, an antisense nucleic acid, a gene, a protein, a peptide, Vpr protein, an enzyme, an intracellular antagonist of HIV, a radionuclide, a cytotoxic compound, an antiviral agent, an imaging agent, or the like.

Inclusion of the additional component in the enveloped virus vector of the disclosure may be accomplished by directly coupling the additional component to the competent portion of the genome of the enveloped virus. For instance, if the competent portion of the genome is provided to the producer cell in the form of a plasmid, the plasmid may comprise a gene encoding an imaging agent, such as luciferase.

Inclusion of the additional component in the enveloped virus vector of the disclosure may also be accomplished by directly coupling the additional component to a nucleic acid encoding the cellular virus receptor protein. For example, if the cellular virus receptor protein is provided to the producer cell in the form of a DNA molecule encoding the same, an additional component comprising a protein may be provided to the producer cell by including the sequence of a gene encoding the protein in the DNA molecule, prior to provision thereof to the producer cell.

The additional component may also be provided directly to the membrane or the cytoplasm of the producer cell by, for example, including the additional component in the extracellular medium of the producer cell.

The producer cell need not normally comprise the desired cellular virus receptor protein or membrane protein of interest. Thus, in another example of making the enveloped virus vector of the disclosure, a producer cell is provided with at least a competent portion of the genome of an enveloped virus and a cellular virus receptor protein/membrane protein of interest, and is thereafter incubated under conditions which permit formation of an enveloped virus vector of the disclosure comprising the cellular virus receptor protein/membrane protein. This method, therefore, does not employ a producer cell which naturally comprises the cellular virus receptor protein or membrane protein of interest.

In this example of making the enveloped virus vector of the disclosure, the manner of providing the cellular virus receptor protein to the producer cell is not critical. By way of example, the cellular virus receptor protein may be provided to the producer cell in the form of a protein associated with the membrane portion of a membrane vesicle, a protein associated with a liposome, a protein associated with the membrane of a cell, a membrane-free solution of the protein, a solid protein, a protein associated with the envelope of an enveloped virus, a protein associated with the envelope of an enveloped virus vector of the disclosure, a nucleic acid, such as DNA or RNA, encoding the protein, a virus, which may be enveloped or non-enveloped, having a nucleic acid which encodes the protein, an enveloped virus vector having a nucleic acid which encodes the protein, or the like. Preferably, the cellular virus receptor protein is provided to the producer cell in the form of a DNA molecule encoding the protein, more preferably in the form of a plasmid. Methods for delivering proteins, membrane vesicles, liposomes, nucleic acids, and viruses to a cell are described in the literature. These methods may be easily adapted to the present situation.

The identity of the cellular virus receptor protein is not critical, except that it should be one which is cognate to a viral envelope protein which is displayed on the surface of a cell with which it is desired to fuse the enveloped virus vector, where applicable. The cellular virus receptor protein may be any protein which is cognate to a viral envelope protein. Preferably, the cellular virus receptor protein is cognate to a retroviral envelope protein, more preferably, it is cognate to a viral envelope protein of a virus selected from the group consisting of HIV, SIV, RSV, and ecotropic MLV. Also preferably, the cellular virus receptor protein is selected from the group consisting of CD4, CCR5, CXCR4, ICAM-1, ICAM-2, ICAM-3, CR3, CR4, CD43, CD44, CD46, CD55, CD59, CD63, CD71, a chemokine receptor, Tva, and MCAT-1. More preferably, the first virus receptor protein is selected from the group consisting of CD4, CCR5, CXCR4, Tva, and MCAT-1. Methods of the disclosure include methods of generating single pool, endogenously homologous viral particle libraries comprising: culturing a cell comprising a composition comprising a nucleic acid sequence comprising:
(a) a first expressible nucleic acid and a second expressible nucleic acid;
(b) a first regulatory sequence operably linked to the first expressible nucleic acid;
(c) a second regulatory sequence operably linked to the second expressible nucleic acid; and
(d) a serine recombinase element encoding a serine recombinase positioned on either the first or the second expressible nucleic acid; wherein (a), (b), (c) and (d) are positioned between a 5′ viral LTR and a 3′ viral LTR; or a cell comprising the nucleic acid sequence.

The following Examples are intended to further illustrate certain embodiment of the disclosure, and are not to be construed in any way as limiting the scope of the disclosure. All references, publications and issued patents disclosed herein are incorporated by reference in their respective entireties.

### Example 1

### Methods

Culture of HEK 293T/17 cells

HEK 293T/17 cells (ATCC, CRL-11268) were maintained in Opti-MEM I Reduced Serum Medium with GlutaMAX Supplement (Thermo Fisher Scientific) supplemented with 5% FBS, 1 mM sodium pyruvate (Thermo Fisher Scientific), and 1× minimal essential medium (MEM) nonessential amino acids (Thermo Fisher Scientific). All procedures involving these cells were performed using this medium unless stated otherwise. Cells were passaged and maintained below 70% confluency.

### Development of HEK 293T/17 Landing Pad Cells

To engineer landing pad cells, HEK 293T/17 cells were electroporated with Cas9 ribonucleoprotein (RNP) and a transfer plasmid carrying the landing pad construct flanked by the AAVS1 left homology (816 bp):

(SEQ ID NO: 51)
tgctttctctgacctgcattctctcccctgggcctgtgccgctttctgtctgcagcttgtggcctgggtcacctctacggctggcccagatcctt
ccctgccgcctccttcaggttccgtcttcctccactccctcttccccttgctctctgctgtgttgctgcccaaggatgctctttccggagcacttc
cttctcggcgctgcaccacgtgatgtcctctgagcggatcctccccgtgtctgggtcctctccgggcatctctcctccctcacccaaccccat
gccgtcttcactcgctgggttcccttttccttctccttctggggcctgtgccatctctcgtttcttaggatggccttctccgacggatgtctccctt
gcgtcccgcctccccttcttgtaggcctgcatcatcaccgtttttctggacaaccccaaagtaccccgtctccctggctttagccacctctcca
tcctcttgctttctttgcctggacaccccgttctcctgtggattcgggtcacctctcactcctttcatttgggcagctcccctaccccccttacctc
tctagtctgtgctagctcttccagccccctgtcatggcatcttccaggggtccgagagctcagctagtcttcttcctccaacccgggcccctat
gtccacttcaggacagcatgtttgctgcctccagggatcctgtgtccccgagctgggaccaccttatattcccagggccggttaatgtggct
ctggttctg ggtacttttatctgtcccctccaccccacagtggggccactagggacag);
and
right homology arms
gattggtgacagaaaagccccatccttaggcctcctccttcctagtctcctgatattgggtctaacccccacctcctgttaggcagattccttat
ctggtgacacacccccatttcctggagccatctctctccttgccagaacctctaaggtttgcttacgatggagccagagaggatcctgggag
ggagagcttggcagggggtgggagggaagggggggatgcgtgacctgcccggttctcagtggccaccctgcgctaccctctcccaga
acctgagctgctctgacgcggccgtctggtgcgtttcactgatcctggtgctgcagcttccttacacttcccaagaggagaagcagtttgga
aaaacaaaatcagaataagttggtcctgagttctaactttggctcttcacctttctagtccccaatttatattgttcctccgtgcgtcagttttacc
tgtgagataaggccagtagccagccccgtcctggcagggctgtggtgaggaggggggtgtccgtgtggaaaactccctttgtgagaatgg
tgcgtcctaggtgttcaccaggtcgtggccgcctctactccctttctctttctccatccttctttccttaaagagtccccagtgctatctgggacat
attcctccgcccagagcagggtcccgcttccctaaggccctgctctgggcttctgggtttgagtccttggcaagcccaggagaggcgctca
ggcttc cctgtcccccttcctcgtccaccatctcatgcccctggctctcctgccccttccctacaggggttcctggctctgctct arms.
(826 bp; (SEQ ID NO: 52))

Cas9 RNP were produced by complexing a human AAVS1-targeting gRNA (targeting sequence: ggggccactagggacaggat (SEQ ID NO: 53)) to Cas9. The gRNA was ordered as a single guide RNA (sgRNA) from IDT and resuspended at 100 μM in IDTE buffer (pH 7.0). Alt-R HiFi Cas9 enzyme (IDT) and the sgRNA were combined in PBS at a final concentration of 24 μM sgRNA and 20.8 μM Cas9. Complex formation was achieved by incubating the mixture at room temperature for 15 minutes.

2×106 HEK 293T/17 cells were resuspended in 20 μL of SF Nucleofector solution (Lonza) and to the cell suspension, 5 μL of RNP solution and 1 μg of transfer plasmid DNA was added. The entire cell suspension was transferred to the electroporation cuvette. Electroporation was done using a Lonza 4D-Nucleofactor X system using the pulse code CM-130 Immediately after electroporation, cells were transferred into a 6-well plate with 2.5 mL of pre-warmed medium containing 0.3 μM HDR Enhancer V2 (IDT). The following day, the culture medium was replaced with fresh medium.

3 days after electroporation, the cells were reseeded in fresh medium containing 10 μg/mL blasticidin (Thermo Fisher Scientific) for selection for approximately two weeks.

After selection, single cell cloning was performed and individual clones were evaluated for functionality.

### Generation of Lentivirus and Adeno-Associated Virus Production Cells

Transfer plasmids carrying the viral payload flanked by attP1 and attP2 were used for integration into the landing pad cells. 3×106 HEK 293T/17 landing pad cells were seeded onto a 12-well plate in 1 mL of medium 12-14 h prior to transfection. For transfection, Lipofectamine 3000 transfection reagent (Thermo Fisher Scientific) was used according to the manufacturer's protocol. Briefly, 1 μg of transfer plasmid DNA and 2 μL of P3000 reagent was added to 50 μL of Opti-MEM I (without added supplements), and 3 μL of Lipofectamine 3000 reagent was added to a separate 50 μL of Opti-MEM I (without added supplements). The two portions were combined and incubated at room temperature for 15 minutes to form the transfection mix. After incubation, the entire volume of transfection mix was added to the previously seeded cells dropwise. 24 hours after transfection, the cells were reseeded onto a 6-well plate in 2.5 mL of fresh medium. After another 48 hours, selection was performed by reseeding the cells in medium supplemented with 10 nM AP1903 (MedChemExpress) and 1 μM trimethoprim (Sigma Aldrich). For experiments involving a larger number of cells, all values were scaled proportionally according to the surface area of the culture vessel.

### Production of Lentivirus Using Landing Pad Cells

6×106 HEK 293T/17 landing pad cells carrying integrated lentiviral construct were seeded onto a 12-well plate in 1 mL of medium 12-14 h prior to transfection. For transfection, Lipofectamine 3000 transfection reagent (Thermo Fisher Scientific) was used according to the manufacturer's protocol. Briefly, 200 ng of psPAX2 (Addgene 12260), 300 ng of pMD2.G (Addgene 12259) or pMD2-VSVGmut (Addgene 182229; for targeted lentivirus), and 1 μL of P3000 reagent was added to 50 μL of Opti-MEM I (without added supplements), and 3 μL of Lipofectamine 3000 reagent was added to a separate 50 μL of Opti-MEM I (without added supplements). The two portions were combined and incubated at room temperature for 15 minutes to form the transfection mix. After incubation, the entire volume of transfection mix was added to the previously seeded cells dropwise. Lentiviral supernatant was collected 48 hours after transfection and centrifuged at 1200 g for 5 min at 4° C., then filtered through a 0.45 μm PES filter to clear cellular debris. Lenti-X Concentrator (Takara Bio) or Lentivirus Precipitation Solution (ALSTEM) was used following manufacturer's protocol and the resulting viral pellet was resuspended in 1% of the original volume of Opti-MEM I (without added supplements). Concentrated lentiviral suspension was stored at −80° C. until use. For production of larger volumes of virus, all values were scaled proportionally according to the surface area of the culture vessel.

### Production of Adeno-Associated Virus Using Landing Pad Cells

Landing pad cells with the viral payload integrated into the AAV landing pad cells were utilized to package vector genomes into different AAV capsids by transfection of HEK293T cells together with adenovirus helper and AAV Rep-Cap plasmids using polyethylenimine (Polysciences #23966). The landing pad cells were seeded in 150 mm plates, each plate was transfected with 6 mg cargo vector, 8 mg of Rep-Cap plasmid, 11 mg of adenovirus helper plasmid in 200 ml PEI for 72 hours. Transfected cells were collected in AAV lysis buffer (50 mM Tris, 150 mM NaCl) and lysed by three rounds of rapid freeze/thawing, followed by a 1 h incubation at 37° C. with 25 units/mL Benzonase (Millipore Sigma #70-664-3). AAV vectors were further purified following cell harvest and PEG precipitation using iodixanol (OptiPrep, StemCell Technologies #07820) gradient ultracentrifugation. AAV vector titers were determined by qPCR on DNaseI (NEB #B0303S) treated, Proteinase K (Qiagen #1114886) digested AAV samples post-purification, using primers targeting the viral genome. qPCR was performed with SsoFast Eva Green Supermix (Bio-Rad #1725201) on a StepOnePlus Real-Time PCR System (Applied Biosystems #4376600). Relative quantity was estimated by comparison to a serial dilution of a vector plasmid standard of known concentration.

### Flow Cytometry-Based Analysis of Recombination

As a proof of concept for the recombination-free nature of the viral particles generated by the method of the disclosure, two constructs carrying two sets of fluorescent proteins were used. In the first construct, mTagBFP2 was paired with mScarlet and in the second, StayGold was paired with emiRFP670. These constructs were combined in a pool and integrated into 293T landing pad cells via site-specific recombination, with only a single copy integrating into each cell. Lentivirus packaging plasmids were subsequently transfected into the 293T landing pads cell for virus production. The virus was collected and used to transduce naive wildtype K562 cells at a low multiplicity of infection (MOI).

In parallel, the same constructs in conventional lentivirus transfer vectors were used to make conventional lentivirus for comparison. The collected virus was used to transduce naïve wildtype K562 cells at a low MOI.

Flow cytometry was used to analyze the transduced cells 3 days after transduction. The frequencies of cells carrying the various combinations of fluorescent proteins were quantified and used to benchmark the performance of the method of the disclosure.

Recombination events led to novel fluorescent protein combinations that were not present in the initial constructs and can be quantified. The combinations are: mTagBFP2-mScarlet (no recombination), StayGold-emiRFP670 (no recombination), mTagBFP2-emiRFP670 (recombination), StayGold-mScarlet (recombination), and all fluorescent proteins (multiple transduction events).

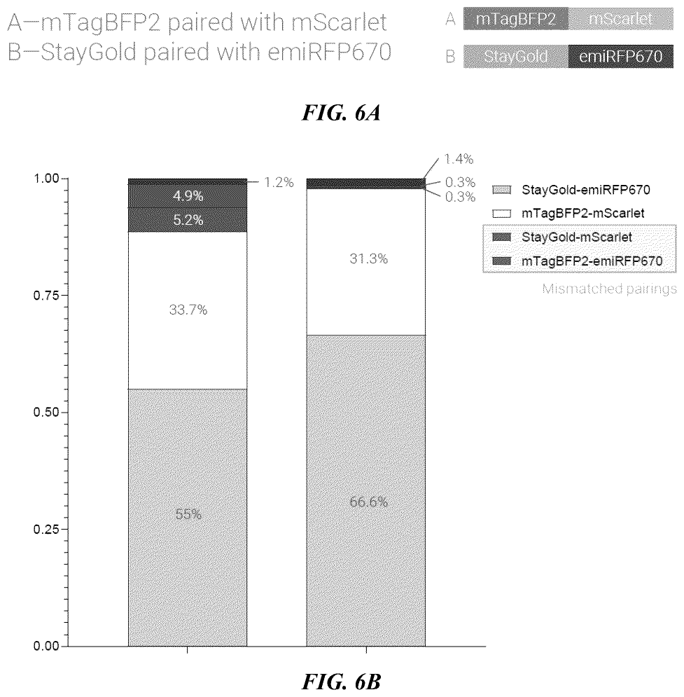

The substrates used are shown in FIG. 6A. Results of the analysis are shown in FIG. 6B (Left=conventional method; right=method of the disclosure. The conventional method resulted in >10% recombination, while the method according to the disclosure resulted in <1% recombination.

### Example 2

### Sequence-Based Testing for Recombination

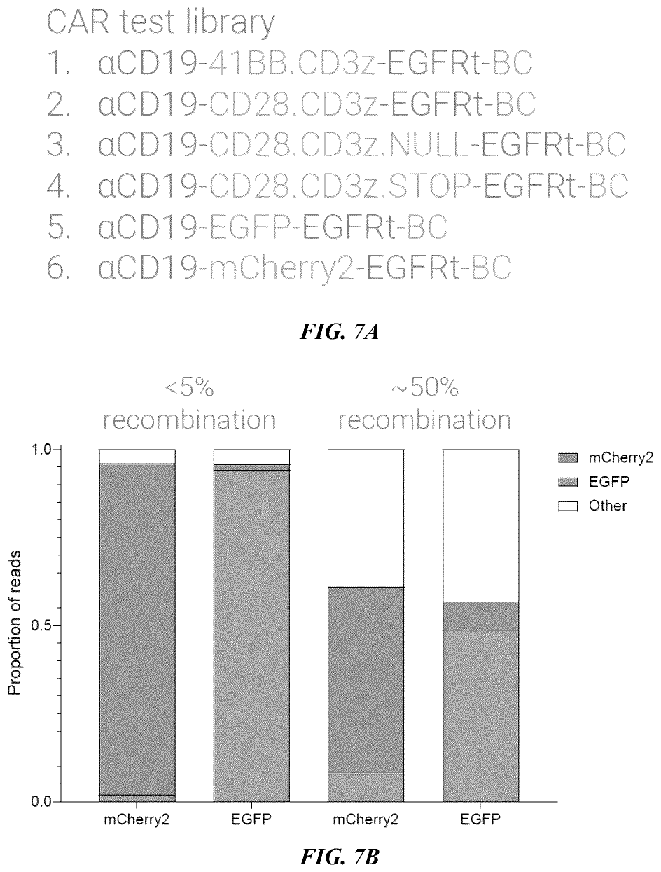

In this study, different chimeric antigen receptors (CARs) were paired with unique barcodes that can be sequenced. In this study, another approach was taken to validate the recombination-free nature of the viral particles. A library of six different chimeric antigen receptors (CARs) were generated and each of them was assigned a unique DNA barcode. Out of the six CAR constructs, one of them contains of a green fluorescent protein (eGFP) and another contains a red fluorescent protein (mCherry2). The method of the disclosure was used to generate lentivirus using this library and the resulting lentivirus was used to transduce K562 cells. The transduced K562 cells were sorted on the basis of fluorescent protein expression into three separate bins: eGFP only, mCherry2 only, and none. DNA was extracted from the sorted cells and polymerase chain reaction (PCR) was used to amplify the barcodes for sequencing. The proportion of barcode reads belonging to each construct was quantified for each sorted bin and used to quantify recombination. The substrates used are shown in FIG. 7A. Infected cells were sorted based on EGFP or mCherry2 and sequenced for the barcode. The results are shown in FIG. 7B. As can be seen the method of the disclosure gave less than 5% recombination (left), whereas the conventional method gave about 50% recombination.

CAR constructs include modification of one or a combination of constructs from Table Z.

TABLE X
Table of CAR constructs

1. αCD19-41BB.CD3z-EGFRt-BC
atggctctcccagtgactgccctactgcttcccctagcgcttctcctgcatgcagaggtgaagctgcagcagtctggggctgagctggtgaggcctgggtcctca
gtgaagatttcctgcaaggcttctggctatgcattcagtagctactggatgaactgggtgaagcagaggcctggacagggtcttgagtggattggacagatttatcc
tggagatggtgatactaactacaatggaaagttcaagggtcaagccacactgactgcagacaaatcctccagcacagcctacatgcagctcagcggcctaacat
ctgaggactctgcggtctatttctgtgcaagaaagaccattagttcggtagtagatttctactttgactactggggccaagggaccacggtcactgtctcctcaggtg
gaggtggatcaggtggaggtggatctggtggaggtggatctgacattgagctcacccagtctccaaaattcatgtccacatcagtaggagacagggtcagcgtc
acctgcaaggccagtcagaatgtgggtactaatgtagcctggtatcaacagaaaccaggacaatctcctaaaccactgatttactcggcaacctaccggaacagt
ggagtccctgatcgcttcacaggcagtggatctgggacagatttcactctcaccatcactaacgtgcagtctaaagacttggcagactatttctgtcaacaatataac
aggtatccgtacacgtccggaggggggaccaagctggagatcaaacggaccacgacgccagcgccgcgaccaccaacaccggcgcccaccatcgcgtcg
cagcctctgtccctgcgcccagaggcgtgccgaccagcggcgggtggagcagtgcacacgagggggctggacttcgcctgtgatatctacatctgggcgccc
ttggccgggacttgtggggtccttctcctgtcactggttatcaccctttactgcaagcggggcagaaagaagctgctgtacatcttcaagcagcccttcatgcggcc
cgtgcagaccacccaggaagaggacggctgctcctgcagattccccgaggaagaagaaggcggctgcgagctgagagtgaagttcagcaggagcgcagac
gcccccgcgtaccagcagggccagaaccagctctataacgagctcaatctaggacgaagagaggagtacgatgttttggacaagaggcgtggccgggaccct
gagatggggggaaagccgagaaggaagaaccctcaggaaggcctgtacaatgaactgcagaaagataagatggcggaggcctacagtgagattgggatga
aaggcgagcgccggaggggcaaggggcacgatggcctttaccagggactcagtacagccaccaaggacacctacgacgcactccatatgcaagcccttcct
ccaagaggaagcggagctactaacttcagcctgctgaagcaggctggtgacgtggaggagaaccctggacccatgcttctcctggtgacaagccttctgctctgt
gagttaccacacccagcattcctcctgatcccacgcaaagtgtgtaacggaataggtattggtgaatttaaagactcactctccataaatgctacgaatattaaacac
ttcaaaaactgcacctccatcagtggcgatctccacatcctgccggtggcatttaggggtgactccttcacacatactcctcctctggacccacaggaactggatatt
ctgaaaaccgtaaaggaaatcacagggtttttgctgattcaggcttggcctgaaaacaggacggacctccatgcctttgagaacctagaaatcatacgcggcagg
accaagcaacatggtcagttttctcttgcagtcgtcagcctgaacataacatccttgggattacgctccctcaaggagataagtgatggagatgtgataatttcagga
aacaaaaatttgtgctatgcaaatacaataaactggaaaaaactgtttgggacctccggtcagaaaaccaaaattataagcaacagaggtgaaaacagctgcaag
gccacaggccaggtctgccatgccttgtgctcccccgagggctgctggggcccggagcccagggactgcgtgtcttgccggaatgtcagccgaggcaggga
atgcgtggacaagtgcaaccttctggagggtgagccaagggagtttgtggagaactctgagtgcatacagtgccacccagagtgcctgcctcaggccatgaac
atcacctgcacaggacggggaccagacaactgtatccagtgtgcccactacattgacggcccccactgcgtcaagacctgcccggcaggagtcatgggagaa
aacaacaccctggtctggaagtacgcagacgccggccatgtgtgccacctgtgccatccaaactgcacctacggatgcactgggccaggtcttgaaggctgtcc
cacgaatgggcctaagatcccgtccatcgccactgggatggtgggggccctcctcttgctgctggtggtggccctggggatcggcctcttcatgggcagcggcg
cgaccaactttagcctgctgaaacaggcgggcgatgttgaagaaaacccaggtcctggntcnggntcnggntcnggntcngggccttcaggtagtcgtgacgt
cgggagtnnknnknnknnknnktcggctgctttaaggccggtcctagcaaagtga (SEQ ID NO: 54)
2. αCD19-CD28.CD3z-EGFRt-BC
atggctctcccagtgactgccctactgcttcccctagcgcttctcctgcatgcagaggtgaagctgcagcagtctggggctgagctggtgaggcctgggtcctcagt
gaagatttcctgcaaggcttctggctatgcattcagtagctactggatgaactgggtgaagcagaggcctggacagggtcttgagtggattggacagatttatcctgg
agatggtgatactaactacaatggaaagttcaagggtcaagccacactgactgcagacaaatcctccagcacagcctacatgcagctcagcggcctaacatctgag
gactctgcggtctatttctgtgcaagaaagaccattagttcggtagtagatttctactttgactactggggccaagggaccacggtcactgtctcctcaggtggaggtgg
atcaggtggaggtggatctggtggaggtggatctgacattgagctcacccagtctccaaaattcatgtccacatcagtaggagacagggtcagcgtcacctgcaag
gccagtcagaatgtgggtactaatgtagcctggtatcaacagaaaccaggacaatctcctaaaccactgatttactcggcaacctaccggaacagtggagtccctgat
cgcttcacaggcagtggatctgggacagatttcactctcaccatcactaacgtgcagtctaaagacttggcagactatttctgtcaacaatataacaggtatccgtacac
gtccggaggggggaccaagctggagatcaaacggaccacgacgccagcgccgcgaccaccaacaccggcgcccaccatcgcgtcgcagcctctgtccctgc
gcccagaggcgtgccgaccagcggcgggtggagcagtgcacacgagggggctggacttcgcctgtgatatctacatctgggcgcccttggccgggacttgtgg
ggtccttctcctgtcactggttatcaccctttactgcgtgaggagtaagaggagcaggctcctgcacagtgactacatgaacatgactccccgccgccccgggccca
cccgcaagcattaccagccctatgccccaccacgcgacttcgcagcctatcgctccagagtgaagttcagcaggagcgcagacgcccccgcgtaccagcaggg
ccagaaccagctctataacgagctcaatctaggacgaagagaggagtacgatgttttggacaagaggcgtggccgggaccctgagatggggggaaagccgaga
aggaagaaccctcaggaaggcctgtacaatgaactgcagaaagataagatggcggaggcctacagtgagattgggatgaaaggcgagcgccggaggggcaag
gggcacgatggcctttaccagggactcagtacagccaccaaggacacctacgacgcactccatatgcaagcccttcctccaagaggaagcggagctactaacttc
agcctgctgaagcaggctggtgacgtggaggagaaccctggacccatgcttctcctggtgacaagccttctgctctgtgagttaccacacccagcattcctcctgatc
ccacgcaaagtgtgtaacggaataggtattggtgaatttaaagactcactctccataaatgctacgaatattaaacacttcaaaaactgcacctccatcagtggcgatct
ccacatcctgccggtggcatttaggggtgactccttcacacatactcctcctctggacccacaggaactggatattctgaaaaccgtaaaggaaatcacagggtttttg
ctgattcaggcttggcctgaaaacaggacggacctccatgcctttgagaacctagaaatcatacgcggcaggaccaagcaacatggtcagttttctcttgcagtcgtc
agcctgaacataacatccttgggattacgctccctcaaggagataagtgatggagatgtgataatttcaggaaacaaaaatttgtgctatgcaaatacaataaactgga
aaaaactgtttgggacctccggtcagaaaaccaaaattataagcaacagaggtgaaaacagctgcaaggccacaggccaggtctgccatgccttgtgctcccccg
agggctgctggggcccggagcccagggactgcgtgtcttgccggaatgtcagccgaggcagggaatgcgtggacaagtgcaaccttctggagggtgagccaag
ggagtttgtggagaactctgagtgcatacagtgccacccagagtgcctgcctcaggccatgaacatcacctgcacaggacggggaccagacaactgtatccagtgt
gcccactacattgacggcccccactgcgtcaagacctgcccggcaggagtcatgggagaaaacaacaccctggtctggaagtacgcagacgccggccatgtgtg
ccacctgtgccatccaaactgcacctacggatgcactgggccaggtcttgaaggctgtcccacgaatgggcctaagatcccgtccatcgccactgggatggtggg
ggccctcctcttgctgctggtggtggccctggggatcggcctcttcatgggcagcggcgcgaccaactttagcctgctgaaacaggcgggcgatgttgaagaaaac
ccaggtcctggntcnggntcnggntcnggntcngggccttcaggtagtcgtgacgtcgggagtnnknnknnknnknn
ktcggctgctttaaggccggtcctagcaaagtga (SEQ ID NO: 55)
3. αCD19-CD28.NULL-EGFRt-BC
atggctctcccagtgactgccctactgcttcccctagcgcttctcctgcatgcagaggtgaagctgcagcagtctggggctgagctggtgaggcctgggtcctcagt
gaagatttcctgcaaggcttctggctatgcattcagtagctactggatgaactgggtgaagcagaggcctggacagggtcttgagtggattggacagatttatcctgg
agatggtgatactaactacaatggaaagttcaagggtcaagccacactgactgcagacaaatcctccagcacagcctacatgcagctcagcggcctaacatctgag
gactctgcggtctatttctgtgcaagaaagaccattagttcggtagtagatttctactttgactactggggccaagggaccacggtcactgtctcctcaggtggaggtgg
atcaggtggaggtggatctggtggaggtggatctgacattgagctcacccagtctccaaaattcatgtccacatcagtaggagacagggtcagcgtcacctgcaag
gccagtcagaatgtgggtactaatgtagcctggtatcaacagaaaccaggacaatctcctaaaccactgatttactcggcaacctaccggaacagtggagtccctgat
cgcttcacaggcagtggatctgggacagatttcactctcaccatcactaacgtgcagtctaaagacttggcagactatttctgtcaacaatataacaggtatccgtacac
gtccggaggggggaccaagctggagatcaaacggaccacgacgccagcgccgcgaccaccaacaccggcgcccaccatcgcgtcgcagcctctgtccctgc
gcccagaggcgtgccgaccagcggcgggtggagcagtgcacacgagggggctggacttcgcctgtgatatctacatctgggcgcccttggccgggacttgtgg
ggtccttctcctgtcactggttatcaccctttactgcgtgaggagtaagaggagcaggctcctgcacagtgactacatgaacatgactccccgccgccccgggccca
cccgcaagcattaccagccctatgccccaccacgcgacttcgcagcctatcgctccagagtgaagttcagcaggagcgcagacgctcctgcctatcagcagggtc
agaaccagcttttcaatgaactgaacctcggcagacgcgaggagtttgatgtgcttgataagcggcgggggcgggaccctgagatggggggaagccaagacgc
aagaatccccaagaggggcttttcaacgaactccagaaagataagatggccgaagctttttcagagatcggcatgaagggtgagcgaaggagggggaagggcc
atgacggtctttttcaaggactgtccacggcaactaaggatactttcgacgcactccatatgcaggcattgccaccacgcggaagcggagctactaacttcagcctgc
tgaagcaggctggtgacgtggaggagaaccctggacccatgcttctcctggtgacaagccttctgctctgtgagttaccacacccagcattcctcctgatcccacgca
aagtgtgtaacggaataggtattggtgaatttaaagactcactctccataaatgctacgaatattaaacacttcaaaaactgcacctccatcagtggcgatctccacatcc
tgccggtggcatttaggggtgactccttcacacatactcctcctctggacccacaggaactggatattctgaaaaccgtaaaggaaatcacagggtttttgctgattcag
gcttggcctgaaaacaggacggacctccatgcctttgagaacctagaaatcatacgcggcaggaccaagcaacatggtcagttttctcttgcagtcgtcagcctgaa
cataacatccttgggattacgctccctcaaggagataagtgatggagatgtgataatttcaggaaacaaaaatttgtgctatgcaaatacaataaactggaaaaaactgt
ttgggacctccggtcagaaaaccaaaattataagcaacagaggtgaaaacagctgcaaggccacaggccaggtctgccatgccttgtgctcccccgagggctgct
ggggcccggagcccagggactgcgtgtcttgccggaatgtcagccgaggcagggaatgcgtggacaagtgcaaccttctggagggtgagccaagggagtttgt
ggagaactctgagtgcatacagtgccacccagagtgcctgcctcaggccatgaacatcacctgcacaggacggggaccagacaactgtatccagtgtgcccacta
cattgacggcccccactgcgtcaagacctgcccggcaggagtcatgggagaaaacaacaccctggtctggaagtacgcagacgccggccatgtgtgccacctgt
gccatccaaactgcacctacggatgcactgggccaggtcttgaaggctgtcccacgaatgggcctaagatcccgtccatcgccactgggatggtgggggccctcc
tcttgctgctggtggtggccctggggatcggcctcttcatgggcagcggcgcgaccaactttagcctgctgaaacagggggcgatgttgaagaaaacccaggtcc
tggntcnggntcnggntcnggntcngggccttcaggtagtcgtgacgtcgggagtnnknnknnknnknnktcggctgctttaaggccggtcctagcaaagtga
(SEQ ID NO: 56)
4. αCD19-CD28.STOP-EGFRt-BC
atggctctcccagtgactgccctactgcttcccctagcgcttctcctgcatgcagaggtgaagctgcagcagtctggggctgagctggtgaggcctgggtcctcagt
gaagatttcctgcaaggcttctggctatgcattcagtagctactggatgaactgggtgaagcagaggcctggacagggtcttgagtggattggacagatttatcctgg
agatggtgatactaactacaatggaaagttcaagggtcaagccacactgactgcagacaaatcctccagcacagcctacatgcagctcagcggcctaacatctgag
gactctgcggtctatttctgtgcaagaaagaccattagttcggtagtagatttctactttgactactggggccaagggaccacggtcactgtctcctcaggtggaggtgg
atcaggtggaggtggatctggtggaggtggatctgacattgagctcacccagtctccaaaattcatgtccacatcagtaggagacagggtcagcgtcacctgcaag
gccagtcagaatgtgggtactaatgtagcctggtatcaacagaaaccaggacaatctcctaaaccactgatttactcggcaacctaccggaacagtggagtccctgat
cgcttcacaggcagtggatctgggacagatttcactctcaccatcactaacgtgcagtctaaagacttggcagactatttctgtcaacaatataacaggtatccgtacac
gtccggaggggggaccaagctggagatcaaacggaccacgacgccagcgccgcgaccaccaacaccggcgcccaccatcgcgtcgcagcctctgtccctgc
gcccagaggcgtgccgaccagcggcgggtggagcagtgcacacgagggggctggacttcgcctgtgatatctacatctgggcgcccttggccgggacttgtgg
ggtccttctcctgtcactggttatcaccctttactgcgtgaggagtaagaggagcaggctcctgcacagtgactaaatgaacatgactccccgccgccccgggccca
cccgcaagcattaccagccctaagccccaccacgcgacttcgcagcctaacgctccagagtgaagttcagcaggagcgcagacgcccccgcgtaacagcaggg
ccagaaccagctctataacgagctcaatctaggacgaagagaggagtacgatgttttggacaagaggcgtggccgggaccctgagatggggggaaagccgaga
aggaagaaccctcaggaaggcctgtacaatgaactgcagaaagataagatggcggaggcctacagtgagattgggatgaaaggcgagcgccggaggggcaag
gggcacgatggcctttaccagggactcagtacagccaccaaggacacctacgacgcactccatatgcaagcccttcctccaagaggaagcggagctactaacttc
agcctgctgaagcaggctggtgacgtggaggagaaccctggacccatgcttctcctggtgacaagccttctgctctgtgagttaccacacccagcattcctcctgatc
ccacgcaaagtgtgtaacggaataggtattggtgaatttaaagactcactctccataaatgctacgaatattaaacacttcaaaaactgcacctccatcagtggcgatct
ccacatcctgccggtggcatttaggggtgactccttcacacatactcctcctctggacccacaggaactggatattctgaaaaccgtaaaggaaatcacagggtttttg
ctgattcaggcttggcctgaaaacaggacggacctccatgcctttgagaacctagaaatcatacgcggcaggaccaagcaacatggtcagttttctcttgcagtcgtc
agcctgaacataacatccttgggattacgctccctcaaggagataagtgatggagatgtgataatttcaggaaacaaaaatttgtgctatgcaaatacaataaactgga
aaaaactgtttgggacctccggtcagaaaaccaaaattataagcaacagaggtgaaaacagctgcaaggccacaggccaggtctgccatgccttgtgctcccccg
agggctgctggggcccggagcccagggactgcgtgtcttgccggaatgtcagccgaggcagggaatgcgtggacaagtgcaaccttctggagggtgagccaag
ggagtttgtggagaactctgagtgcatacagtgccacccagagtgcctgcctcaggccatgaacatcacctgcacaggacggggaccagacaactgtatccagtgt
gcccactacattgacggcccccactgcgtcaagacctgcccggcaggagtcatgggagaaaacaacaccctggtctggaagtacgcagacgccggccatgtgtg
ccacctgtgccatccaaactgcacctacggatgcactgggccaggtcttgaaggctgtcccacgaatgggcctaagatcccgtccatcgccactgggatggtggg
ggccctcctcttgctgctggtggtggccctggggatcggcctcttcatgggcagcggcgcgaccaactttagcctgctgaaacaggcgggcgatgttgaagaaaac
ccaggtcctggntcnggntcnggntcnggntcngggccttcaggtagtcgtgacgtcgggagtnnknnknnknnknnktcggctgctttaaggccggtcctag
caaagtga (SEQ ID NO: 57)
5. αCD19-CD28.EGFP-EGFRt-BC
atggctctcccagtgactgccctactgcttcccctagcgcttctcctgcatgcagaggtgaagctgcagcagtctggggctgagctggtgaggcctgggtcctcagt
gaagatttcctgcaaggcttctggctatgcattcagtagctactggatgaactgggtgaagcagaggcctggacagggtcttgagtggattggacagatttatcctgg
agatggtgatactaactacaatggaaagttcaagggtcaagccacactgactgcagacaaatcctccagcacagcctacatgcagctcagcggcctaacatctgag
gactctgcggtctatttctgtgcaagaaagaccattagttcggtagtagatttctactttgactactggggccaagggaccacggtcactgtctcctcaggtggaggtgg
atcaggtggaggtggatctggtggaggtggatctgacattgagctcacccagtctccaaaattcatgtccacatcagtaggagacagggtcagcgtcacctgcaag
gccagtcagaatgtgggtactaatgtagcctggtatcaacagaaaccaggacaatctcctaaaccactgatttactcggcaacctaccggaacagtggagtccctgat
cgcttcacaggcagtggatctgggacagatttcactctcaccatcactaacgtgcagtctaaagacttggcagactatttctgtcaacaatataacaggtatccgtacac
gtccggaggggggaccaagctggagatcaaacggaccacgacgccagcgccgcgaccaccaacaccggcgcccaccatcgcgtcgcagcctctgtccctgc
gcccagaggcgtgccgaccagcggcgggtggagcagtgcacacgagggggctggacttcgcctgtgatatctacatctgggcgcccttggccgggacttgtgg
ggtccttctcctgtcactggttatcaccctttactgcatggtgagcaagggcgaggagctgttcaccggggtggtgcccatcctggtcgagctggacggcgacgtaa
acggccacaagttcagcgtgtccggcgagggcgagggcgatgccacctacggcaagctgaccctgaagttcatctgcaccaccggcaagctgcccgtgccctg
gcccaccctcgtgaccaccctgacctacggcgtgcagtgcttcagccgctaccccgaccacatgaagcagcacgacttcttcaagtccgccatgcccgaaggcta
cgtccaggagcgcaccatcttcttcaaggacgacggcaactacaagacccgcgccgaggtgaagttcgagggcgacaccctggtgaaccgcatcgagctgaag
ggcatcgacttcaaggaggacggcaacatcctggggcacaagctggagtacaactacaacagccacaacgtctatatcatggccgacaagcagaagaacggcat
caaggtgaacttcaagatccgccacaacatcgaggacggcagcgtgcagctcgccgaccactaccagcagaacacccccatcggcgacggccccgtgctgctg
cccgacaaccactacctgagcacccagtccgccctgagcaaagaccccaacgagaagcgcgatcacatggtcctgctggagttcgtgaccgccgccgggatca
ctctcggcatggacgagctgtacaagggaagcggagctactaacttcagcctgctgaagcaggctggtgacgtggaggagaaccctggacccatgcttctcctgg
tgacaagccttctgctctgtgagttaccacacccagcattcctcctgatcccacgcaaagtgtgtaacggaataggtattggtgaatttaaagactcactctccataaatg
ctacgaatattaaacacttcaaaaactgcacctccatcagtggcgatctccacatcctgccggtggcatttaggggtgactccttcacacatactcctcctctggaccca
caggaactggatattctgaaaaccgtaaaggaaatcacagggtttttgctgattcaggcttggcctgaaaacaggacggacctccatgcctttgagaacctagaaatc
atacgcggcaggaccaagcaacatggtcagttttctcttgcagtcgtcagcctgaacataacatccttgggattacgctccctcaaggagataagtgatggagatgtg
ataatttcaggaaacaaaaatttgtgctatgcaaatacaataaactggaaaaaactgtttgggacctccggtcagaaaaccaaaattataagcaacagaggtgaaaac
agctgcaaggccacaggccaggtctgccatgccttgtgctcccccgagggctgctggggcccggagcccagggactgcgtgtcttgccggaatgtcagccgag
gcagggaatgcgtggacaagtgcaaccttctggagggtgagccaagggagtttgtggagaactctgagtgcatacagtgccacccagagtgcctgcctcaggcc
atgaacatcacctgcacaggacggggaccagacaactgtatccagtgtgcccactacattgacggcccccactgcgtcaagacctgcccggcaggagtcatggg
agaaaacaacaccctggtctggaagtacgcagacgccggccatgtgtgccacctgtgccatccaaactgcacctacggatgcactgggccaggtcttgaaggctg
tcccacgaatgggcctaagatcccgtccatcgccactgggatggtgggggccctcctcttgctgctggtggtggccctggggatcggcctcttcatgggcagcggc
gcgaccaactttagcctgctgaaacagggggcgatgttgaagaaaacccaggtcctggntcnggntcnggntcnggntcngggccttcaggtagtcgtgacgt
cgggagtnnknnknnknnknnktcggctgctttaaggccggtcctagcaaagtga (SEQ ID NO: 58)
6. αCD19-CD28.mCherry2-EGFRt-BC
atggctctcccagtgactgccctactgcttcccctagcgcttctcctgcatgcagaggtgaagctgcagcagtctggggctgagctggtgaggcctgggtcctcagt
gaagatttcctgcaaggcttctggctatgcattcagtagctactggatgaactgggtgaagcagaggcctggacagggtcttgagtggattggacagatttatcctgg
agatggtgatactaactacaatggaaagttcaagggtcaagccacactgactgcagacaaatcctccagcacagcctacatgcagctcagcggcctaacatctgag
gactctgcggtctatttctgtgcaagaaagaccattagttcggtagtagatttctactttgactactggggccaagggaccacggtcactgtctcctcaggtggaggtgg
atcaggtggaggtggatctggtggaggtggatctgacattgagctcacccagtctccaaaattcatgtccacatcagtaggagacagggtcagcgtcacctgcaag
gccagtcagaatgtgggtactaatgtagcctggtatcaacagaaaccaggacaatctcctaaaccactgatttactcggcaacctaccggaacagtggagtccctgat
cgcttcacaggcagtggatctgggacagatttcactctcaccatcactaacgtgcagtctaaagacttggcagactatttctgtcaacaatataacaggtatccgtacac
gtccggaggggggaccaagctggagatcaaacggaccacgacgccagcgccgcgaccaccaacaccggcgcccaccatcgcgtcgcagcctctgtccctgc
gcccagaggcgtgccgaccagcggcgggtggagcagtgcacacgagggggctggacttcgcctgtgatatctacatctgggcgcccttggccgggacttgtgg
ggtccttctcctgtcactggttatcaccctttactgcatggtgagcaagggcgaggaggataacatggccatcatcaaggagttcatgcgcttcaaggtgcacatgga
gggctccgtgaacggccacgagttcgagatcgagggcgagggcgagggccgcccctacgagggcacccagaccgccaagctgaaggtgaccaagggggc
cccctgcccttcgcctgggacatcctgtcccctcagttcatgtacggctccaaggcctacgtgaagcaccccgccgacatccccgactacttgaagctgtccttcccc
gagggcttcaattgggagcgcgtgatgaacttcgaggacggcggcgtggtgaccgtgacccaggactcctccctgcaggacggcgagttcatctacaaggtgaa
gctgcgcggcaccaacttcccctccgacggccccgtaatgcagtgtcgtaccatgggctgggaggcctccactgagcggatgtaccccgaggacggcgccctga
agggcgagatcaagcagaggctgaagctgaaggacggcggccactacgacgctgaggtcaagaccacctacaaggccaagaagcccgtgcagctgcccggc
gcctacaacgtcgacatcaagttggacatcctttcccacaacgaggactacaccatgtggaacagtacgaacgcgccgagggccgccactccaccggcggcat
ggacgagctgtacaagggaagcggagctactaacttcagcctgctgaagcaggctggtgacgtggaggagaaccctggacccatgcttctcctggtgacaagcct
tctgctctgtgagttaccacacccagcattcctcctgatcccacgcaaagtgtgtaacggaataggtattggtgaatttaaagactcactctccataaatgctacgaatat
taaacacttcaaaaactgcacctccatcagtggcgatctccacatcctgccggtggcatttaggggtgactccttcacacatactcctcctctggacccacaggaactg
gatattctgaaaaccgtaaaggaaatcacagggtttttgctgattcaggcttggcctgaaaacaggacggacctccatgcctttgagaacctagaaatcatacgcggc
aggaccaagcaacatggtcagttttctcttgcagtcgtcagcctgaacataacatccttgggattacgctccctcaaggagataagtgatggagatgtgataatttcag
gaaacaaaaatttgtgctatgcaaatacaataaactggaaaaaactgtttgggacctccggtcagaaaaccaaaattataagcaacagaggtgaaaacagctgcaag
gccacaggccaggtctgccatgccttgtgctcccccgagggctgctggggcccggagcccagggactgcgtgtcttgccggaatgtcagccgaggcagggaat
gcgtggacaagtgcaaccttctggagggtgagccaagggagtttgtggagaactctgagtgcatacagtgccac
ccagagtgcctgcctcaggccatgaacatcacctgcacaggacggggaccagacaactgtatccagtgtgcccactacattgacggcccccactgcgtcaagacc
tgcccggcaggagtcatgggagaaaacaacaccctggtctggaagtacgcagacgccggccatgtgtgccacctgtgccatccaaactgcacctacggatgcact
gggccaggtcttgaaggctgtcccacgaatgggcctaagatcccgtccatcgccactgggatggtgggggccctcctcttgctgctggtggtggccctggggatcg
gcctcttcatgggcagcggcgcgaccaactttagcctgctgaaacaggcgggcgatgttgaagaaaacccaggtcctggntcnggntcnggntcnggntcngg
gccttcaggtag tcgtgacgtcgggagtnnknnknnknnknnktcggctgctttaaggccggtcctagcaaagtga (SEQ ID NO: 59)

### Example 3 (Prophetic)

### Pooled Production of Reprogrammed Lentivirus

Lentiviruses have been pseudotyped through various strategies, enhancing their utility in biotechnology and as vectors in gene therapy. The Vesicular Stomatitis Virus G protein (VSVG) is notable for its robustness and capability to infect diverse cell types, making it a preferred choice for lab studies and cell manufacturing for clinical use. It's been demonstrated that by introducing K47Q and R354A mutations to VSVG, and co-expressing a targeting receptor, lentivirus can be re-directed to specific cell types, expanding the possibilities for targeted gene delivery.

The method of the disclosure can be utilized to generate clonal re-targeted lentiviral particles. This will be achieved in several steps: 1) pooled transfection of the library onto the landing pad cells, 2) single-copy integration of a library element into each landing pad cell, and 3) transfection of viral packaging mix containing mutant VSVG. Each landing pad cell will effectively only produce viral particles directed by the single library element that is integrated into its genome and pooled production of heterogenous, but clonal, virus is achieved via this process in a single large pool.

### Example 4

### Methods

### Culture of 293F Landing Pad Cells in Suspension

HEK 293F cells engineered with landing pad were maintained in LV-MAX production medium (Thermo Fisher Scientific) in sterile flasks in a shaking incubator at 37° C., 8% CO₂. All procedures involving these cells were performed using this medium unless stated otherwise. Cells were passaged and maintained at a density of 0.5-6×10⁶ cells per mL of culture medium.

### Generation of VLP Production Cells in Adherent Culture

Transfer plasmids carrying the VLP construct flanked by attP1 and attP2 were used for integration into the landing pad cells. 3×10⁶ HEK 293T/17 landing pad cells were seeded onto a 12-well plate in 1 mL of medium 12-14 h prior to transfection. For transfection, Lipofectamine 3000 transfection reagent (Thermo Fisher Scientific) was used according to the manufacturer's protocol. Briefly, 1 μg of transfer plasmid DNA and 2 μL of P3000 reagent was added to 50 μL of Opti-MEM I (without added supplements), and 3 μL of Lipofectamine 3000 reagent was added to a separate 50 μL of Opti-MEM I (without added supplements). The two portions were combined and incubated at room temperature for 15 minutes to form the transfection mix. After incubation, the entire volume of transfection mix was added to the previously seeded cells dropwise. 24 h after transfection, the cells were reseeded onto a 6-well plate in 2.5 mL of fresh medium. After another 48 h, selection was performed by reseeding the cells in medium supplemented with 10 nM AP1903 (MedChemExpress) and 1 μM trimethoprim (Sigma Aldrich). For experiments involving a larger number of cells, all values were scaled proportionally according to the surface area of the culture vessel.

### Generation of VLP Production Cells in Suspension Culture

Transfer plasmids carrying the VLP construct flanked by attP1 and attP2 were used for integration into the landing pad cells. Cell suspension containing 2× 10⁶ HEK 293F landing pad cells per mL was prepared in LV-MAX production medium (Thermo Fisher Scientific). Transfection was performed using LV-MAX transfection reagent (Thermo Fisher Scientific). For every mL of cell suspension to be transfected, 1.25 μg of transfer plasmid DNA was added to 50 μL of Opti-MEM I (without added supplements), and 6 μL of LV-MAX transfection reagent was added to a separate 50 μL of Opti-MEM I (without added supplements). The two portions were combined and incubated at room temperature for 15 minutes to form the transfection mix. After incubation, the entire volume of transfection mix was added directly to the cell suspension. 48 h after transfection, the cells were pelleted and resuspended in twice the initial starting volume of fresh LV-MAX medium. After another 24 h, selection was performed by adding 10 nM AP1903 (MedChemExpress) and 1 μM trimethoprim (Sigma Aldrich).

Production of VLPs with Capsid Using Landing Pad Cells in Adherent Culture

6×10⁶ HEK 293T/17 landing pad cells carrying integrated VLP construct were seeded onto a 12-well plate in 1 mL of medium 12-14 h prior to transfection. For transfection, Lipofectamine 3000 transfection reagent (Thermo Fisher Scientific) was used according to the manufacturer's protocol. Briefly, 600 ng of plasmid with viral capsid and 1.2 μL of P3000 reagent was added to 50 μL of Opti-MEM I (without added supplements), and 3 μL of Lipofectamine 3000 reagent was added to a separate 50 μL of Opti-MEM I (without added supplements). The two portions were combined and incubated at room temperature for 15 minutes to form the transfection mix. After incubation, the entire volume of transfection mix was added to the previously seeded cells dropwise. Cell culture supernatant was collected 48 h after transfection and centrifuged at 1200 g for 5 min at 4° C., then filtered through a 0.45 μm PES filter to clear cellular debris. Lenti-X Concentrator (Takara Bio) or Lentivirus Precipitation Solution (ALSTEM) was used following manufacturer's protocol and the resulting pellet was resuspended in 1% of the original volume of Opti-MEM I (without added supplements). For production of larger volumes of VLPs, all values were scaled proportionally according to the surface area of the culture vessel.

### Production of Self-Contained VLPs Using Landing Pad Cells in Adherent Culture

6×10⁶ HEK 293T/17 landing pad cells carrying integrated VLP construct were seeded onto a 12-well plate in 1 mL of medium and cultured for 48 h. Cell culture supernatant was collected 48 h after transfection and centrifuged at 1200 g for 5 min at 4° C., then filtered through a 0.45 μm PES filter to clear cellular debris. Lenti-X Concentrator (Takara Bio) or Lentivirus Precipitation Solution (ALSTEM) was used following manufacturer's protocol and the resulting pellet was resuspended in 1% of the original volume of Opti-MEM I (without added supplements). For production of larger volumes of VLPs, all values were scaled proportionally according to the surface area of the culture vessel.

Production of VLPs with Capsid Using Landing Pad Cells in Suspension Culture

Landing pad cells with the VLP construct integrated into landing pad cells were utilized for particle production. Cell suspension containing 4×10⁶ HEK 293F landing pad cells per mL was prepared in LV-MAX production medium with LV-MAX supplement (Thermo Fisher Scientific). Transfection was performed using LV-MAX transfection reagent (Thermo Fisher Scientific). For every mL of cell suspension to be transfected, 1.5 μg of capsid plasmid DNA was added to 50 μL of Opti-MEM I (without added supplements), and 6 μL of LV-MAX transfection reagent was added to a separate 50 μL of Opti-MEM I (without added supplements). The two portions were combined and incubated at room temperature for 15 minutes to form the transfection mix. After incubation, the entire volume of transfection mix was added directly to the cell suspension. Cell culture supernatant was collected 48 h after transfection and centrifuged at 1200 g for 5 min at 4° C., then filtered through a 0.45 μm PES filter to clear cellular debris. Lenti-X Concentrator (Takara Bio) or Lentivirus Precipitation Solution (ALSTEM) was used following manufacturer's protocol and the resulting pellet was resuspended in 1% of the original volume of Opti-MEM I (without added supplements).

### Production of Self-Contained VLPs Using Landing Pad Cells in Suspension Culture

Landing pad cells with the VLP construct integrated into landing pad cells were utilized for particle production. Cell suspension containing 4×10⁶ HEK 293F landing pad cells per mL was prepared in LV-MAX production medium with LV-MAX supplement (Thermo Fisher Scientific) and cultured for 48 h. Cell culture supernatant was collected 48 h after transfection and centrifuged at 1200 g for 5 min at 4° C., then filtered through a 0.45 μm PES filter to clear cellular debris. Lenti-X Concentrator (Takara Bio) or Lentivirus Precipitation Solution (ALSTEM) was used following manufacturer's protocol and the resulting pellet was resuspended in 1% of the original volume of Opti-MEM I (without added supplements).

### Example 5

Exemplary sequences of components are provided in the following Table AA. Some of the below components or a combination thereof are a payload in some embodiments. Some embodiments comprise one or more of the following components or a functional variant thereof having about 70%, about 75%, about 80%, about 85%, about 90%, about 91%, about 92%, about 93%, about 94%, about 95%, about 96%, about 97%, about 98%, or about 99% sequence identity to SEQ ID NOS: 67-86. Some embodiments comprise one or more of the following components or a functional variant thereof having about 70%, about 75%, about 80%, about 85%, about 90%, about 91%, about 92%, about 93%, about 94%, about 95%, about 96%, about 97%, about 98%, or about 99% sequence identity to the particular component of Table AA or combination thereof.

TABLE AA

| Component | Variant | DNA Sequence | Amino Acid Sequence |
|---|---|---|---|
| Capsid | EPN24 | | MHGLQDDPDLQALLKGSQLLKVKSSSWRRERFY |
| | | | KLQEDCKTIWQESRKVMRSPESQLFSIEDIQEVRM |
| | | | GHRTEGLEKFARDIPEDRCFSIVFKDQRNTLDLIA |
| | | | PSPADAQHWVQGLRKIIHHSGSMDQRQKLQSRPE |
| | | | PTAPPEESFRSGVETTTPPQKQEPIDKELYPLTSLR |
| | | | SLFGNDPSSQKIEELFKKHKIVAVLRANSVEEAKK |
| | | | KALAVFLGGVHLIEITFTVPDADTVIKELSFLKEM |
| | | | GAIIGAGTVTSVEQCRKAVESGAEFIVSPHLDEEIS |
| | | | QFCKEKGVFYMPGVMTPTELVKAMKLGHTILKL |
| | | | FPGEVVGPQFVKAMKGPFPNVKFVPTGGVNLDN |
| | | | VCEWFKAGVLAVGVGSALVKGTPVEVAEKAKA |
| | | | FVEKIRGCT (SEQ ID NO: 60) |
| Capsid | HIV Gag | | MGARASVLSGGELDRWEKIRLRPGGKKKYKLKH |
| | | | IVWASRELERFAVNPGLLETSEGCRQILGQLQPSL |
| | | | QTGSEELRSLYNTVATLYCVHQRIEIKDTKEALD |
| | | | KIEEEQNKSKKKAQQAAADTGHSNQVSQNYPIV |
| | | | QNIQGQMVHQAISPRTLNAWVKVVEEKAFSPEVI |
| | | | PMFSALSEGATPQDLNTMLNTVGGHQAAMQML |
| | | | KETINEEAAEWDRVHPVHAGPIAPGQMREPRGSD |
| | | | IAGTTSTLQEQIGWMTNNPPIPVGEIYKRWIILGLN |
| | | | KIVRMYSPTSILDIRQGPKEPFRDYVDRFYKTLRA |
| | | | EQASQEVKNWMTETLLVQNANPDCKTILKALGP |
| | | | AATLEEMMTACQGVGGPGHKARVLAEAMSQVT |
| | | | NSATIMMQRGNFRNQRKIVKCFNCGKEGHTARN |
| | | | CRAPRKKGCWKCGKEGHQMKDCTERQANFLGK |
| | | | IWPSYKGRPGNFLQSRPEPTAPPEESFRSGVETTTP |
| | | | PQKQEPIDKELYPLTSLRSLFGNDPSSQ (SEQ ID |
| | | | NO: 61) |
| | | | MGQTVTTPLSLTLGHWKDVERIAHNQSVDVKKR |
| Capsid | MMLV | | RWVTFCSAEWPTFNVGWPRDGTFNRDLITQVKIK |
| | Gag | | VFSPGPHGHPDQVPYIVTWEALAFDPPPWVKPFV |
| | | | HPKPPPPLPPSAPSLPLEPPRSTPPRSSLYPALTPSL |
| | | | GAKPKPQVLSDSGGPLIDLLTEDPPPYRDPRPPPS |
| | | | DRDGNGGEATPAGEAPDPSPMASRLRGRREPPVA |
| | | | DSTTSQAFPLRAGGNGQLQYWPFSSSDLYNWKN |
| | | | NNPSFSEDPGKLTALIESVLITHQPTWDDCQQLLG |
| | | | TLLTGEEKQRVLLEARKAVRGDDGRPTQLPNEV |
| | | | DAAFPLERPDWDYTTQAGRNHLVHYRQLLLAGL |
| | | | QNAGRSPTNLAKVKGITQGPNESPSAFLERLKEA |
| | | | YRRYTPYDPEDPGQETNVSMSFIWQSAPDIGRKL |
| | | | ERLEDLKNKTLGDLVREAEKIFNKRETPEEREERI |
| | | | RRETEEKEERRRTEDEQKEKERDRRRHREMSKLL |
| | | | ATVVSGQKQDRQGGERRRSQLDRDQCAYCKEK |
| | | | GHWAKDCPKKPRGPRGPRPQTSLL (SEQ ID NO: |
| | | | 62) |
| Export | EABR | | FNSSINNIHEMEIQLKDALEKNQQWLVYDQQREV |
| signal | | | YVKGLLAKIFELEKKTETAAHSLP (SEQ ID NO: |
| | | | 630) |
| Export | EPM- | | ALPGNPDHREMGETLPEEVGEYRQPSGGSVPVSP |
| signal | EABR | | GPPSGLEPTSSSPYGGGSFNSSINNIHEMEIQLKDA |
| | | | LEKNQQWLVYDQQREVYVKGLLAKIFELEKKTE |
| | | | TAAHSLP (SEQ ID NO: 64) |
| Export | p6 | | LQSRPEPTAPPEESFRSGVETTTPPQKQEPIDKELY |
| signal | | | PLTSLRSLFGNDPSSQ (SEQ ID NO: 65) |
| RNA | MS2 coat | | ASNFTQFVLVDNGGTGDVTVAPSNFANGIAEWIS |
| binding | protein | | SNSRSQAYKVTCSVRQSSAQKRKYTIKVEVPKGA |
| domain | (variant | | WRSYLNMELTIPIFATNSDCELIVKAMQGLLKDG |
| | 1) | | NPIPSAIAANSGIY (SEQ ID NO: 66) |
| RNA | MS2 coat | | ASNFTQFVLVDNGGTGDVTVAPSNFANGVAEWI |
| binding | protein | | SSNSRSQAYKVTCSVROSSAQKRKYTIKVEVPKV |
| domain | (variant | | ATQTVGGVELPVAAWRSYLNMELTIPIFATNSDC |
| | 2) | | ELIVKAMQGLLKDGNPIPSAIAANSGIY (SEQ ID |
| | | | NO: 67) |
| RNA | PP7 coat | | MGSKTIVLSVGEATRTLTEIQSTADRQIFEEKVGP |
| binding | protein | | LVGRLRLTASLRQNGAKTAYRVNLKLDQADVVD |
| domain | | | SGLPKVRYTQVWSHDVTIVANSTEASRKSLYDLT |
| | | | KSLVATSQVEDLVVNLVPLGR (SEQ ID NO: 68) |
| Surface | Anti- | | MALPVTALLLPLALLLHAARPDIVLTQSPPSLAMS |
| display | BCMA | | LGKRATISCRASESVTILGSHLIHWYQQKPGQPPT |
| protein | scFv | | LLIQLASNVQTGVPARFSGSGSRTDFTLTIDPVEE |
| (antibody | | | DDVAVYYCLQSRTIPRTFGGGTKLEIKGSTSGSGK |
| scFv) | | | PGSGEGSTKGQIQLVQSGPELKKPGETVKISCKAS |
| | | | GYTFTDYSINWVKRAPGKGLKWMGWINTETREP |
| | | | AYAYDFRGRFAFSLETSASTAYLQINNLKYEDTA |
| | | | TYFCALDYSYAMDYWGQGTSVTVSSAAA (SEQ |
| | | | ID NO: 69) |
| Surface | Anti- | | MALPVTALLLPLALLLHAEVKLQQSGAELVRPGS |
| display | CD19 | | SVKISCKASGYAFSSYWMNWVKQRPGQGLEWIG |
| protein | scFv | | QIYPGDGDTNYNGKFKGQATLTADKSSSTAYMQ |
| (antibody | | | LSGLTSEDSAVYFCARKTISSVVDFYFDYWGQGT |
| scFv) | | | TVTVSSGGGGSGGGGSGGGGSDIELTQSPKFMST |
| | | | SVGDRVSVTCKASQNVGTNVAWYQQKPGQSPKP |
| | | | LIYSATYRNSGVPDRFTGSGSGTDFTLTITNVQSK |
| | | | DLADYFCQQYNRYPYTSGGGTKLEIKR (SEQ ID |
| | | | NO: 70) |
| Surface | CCR5 | | MDYQVSSPIYDINYYTSEPCQKINVKQIAARLLPP |
| display | | | LYSLVFIFGFVGNMLVILILINCKRLKSMTDIYLLN |
| protein | | | LAISDLFFLLTVPFWAHYAAAQWDFGNTMCQLL |
| (G- | | | TGLYFIGFFSGIFFIILLTIDRYLAVVHAVFALKART |
| protein | | | VTFGVVTSVITWVVAVFASLPGIIFTRSQKEGLHY |
| coupled | | | TCSSHFPYSQYQFWKNFQTLKIVILGLVLPLLVMV |
| receptor) | | | ICYSGILKTLLRCRNEKKRHRAVRLIFTIMIVYFLF |
| | | | WAPYNIVLLLNTFQEFFGLNNCSSSNRLDQAMQV |
| | | | TETLGMTHCCINPIIYAFVGEKFRNYLLVFFQKHI |
| | | | AKRFCKCCSIFQQEAPERASSVYTRSTGEQEISVG |
| | | | L (SEQ ID NO: 71) |
| Surface | CXCR4 | | MEGISIYTSDNYTEEMGSGDYDSMKEPCFREENA |
| display | | | NFNKIFLPTIYSIIFLTGIVGNGLVILVMGYQKKLR |
| protein | | | SMTDKYRLHLSVADLLFVITLPFWAVDAVANWY |
| (G- | | | FGNFLCKAVHVIYTVNLYSSVLILAFISLDRYLAIV |
| protein | | | HATNSQRPRKLLAEKVVYVGVWIPALLLTIPDFIF |
| coupled | | | ANVSEADDRYICDRFYPNDLWVVVFQFQHIMVG |
| receptor) | | | LILPGIVILSCYCIIISKLSHSKGHQKRKALKTTVILI |
| | | | LAFFACWLPYYIGISIDSFILLEIIKQGCEFENTVHK |
| | | | WISITEALAFFHCCLNPILYAFLGAKFKTSAQHAL |
| | | | TSVSRGSSLKILSKGKRGGHSSVSTESESSSFHSS |
| | | | (SEQ ID NO: 72) |
| Surface | HTR1F | | MDFLNSSDQNLTSEELLNRMPSKILVSLTLSGLAL |
| display | | | MTTTINSLVIAAIIVTRKLHHPANYLICSLAVTDFL |
| protein | | | VAVLVMPFSIVYIVRESWIMGQVVCDIWLSVDIT |
| (G- | | | CCTCSILHLSAIALDRYRAITDAVEYARKRTPKHA |
| protein | | | GIMITIVWIISVFISMPPLFWRHQGTSRDDECIIKHD |
| coupled | | | HIVSTIYSTFGAFYIPLALILILYYKIYRAAKTLYHK |
| receptor) | | | RQASRIAKEEVNGQVLLESGEKSTKSVSTSYVLE |
| | | | KSLSDPSTDFDKIHSTVRSLRSEFKHEKSWRRQKI |
| | | | SGTRERKAATTLGLILGAFVICWLPFFVKELVVNV |
| | | | CDKCKISEEMSNFLAWLGYLNSLINPLIYTIFNEDF |
| | | | KKAFQKLVRCRC (SEQ ID NO: 73) |
| Surface | CD274 | | MRIFAVFIFMTYWHLLNAFTVTVPKDLYVVEYGS |
| display | (PDL1) | | NMTIECKFPVEKQLDLAALIVYWEMEDKNIIQFV |
| protein | | | HGEEDLKVQHSSYRQRARLLKDQLSLGNAALQIT |
| (ligand) | | | DVKLQDAGVYRCMISYGGADYKRITVKVNAPYN |
| | | | KINQRILVVDPVTSEHELTCQAEGYPKAEVIWTSS |
| | | | DHQVLSGKTTTTNSKREEKLFNVTSTLRINTTTNE |
| | | | IFYCTFRRLDPEENHTAELVIPELPLAHPPNERTHL |
| | | | VILGAILLCLGVALTFIFRLRKGRMMDVKKCGIQ |
| | | | DTNSKKQSDTHLEET (SEQ ID NO: 74) |
| Surface | CD80 | | MGHTRRQGTSPSKCPYLNFFQLLVLAGLSHFCSG |
| display | | | VIHVTKEVKEVATLSCGHNVSVEELAQTRIYWQK |
| protein | | | EKKMVLTMMSGDMNIWPEYKNRTIFDITNNLSIV |
| (ligand) | | | ILALRPSDEGTYECVVLKYEKDAFKREHLAEVTL |
| | | | SVKADFPTPSISDFEIPTSNIRRIICSTSGGFPEPHLS |
| | | | WLENGEELNAINTTVSQDPETELYAVSSKLDENM |
| | | | TTNHSFMCLIKYGHLRVNQTFNWNTTKQEHFPD |
| | | | NLLPSWAITLISVNGIFVICCLTYCFAPRCRERRRN |
| | | | ERLRRESVRPV (SEQ ID NO: 75) |
| Surface | CD86 | | MDPQCTMGLSNILFVMAFLLSGAAPLKIQAYFNE |
| display | | | TADLPCQFANSQNQSLSELVVFWQDQENLVLNE |
| protein | | | VYLGKEKFDSVHSKYMGRTSFDSDSWTLRLHNL |
| (ligand) | | | QIKDKGLYQCIIHHKKPTGMIRIHQMNSELSVLAN |
| | | | FSQPEIVPISNITENVYINLTCSSIHGYPEPKKMSVL |
| | | | LRTKNSTIEYDGVMQKSQDNVTELYDVSISLSVSF |
| | | | PDVTSNMTIFCILETDKTRLLSSPFSIELEDPQPPPD |
| | | | HIPWITAVLPTVIICVMVFCLILWKWKKKKRPRNS |
| | | | YKCGTNTMEREESEQTKKREKIHIPERSDEAQRVF |
| | | | KSSKTSSCDKSDTCF (SEQ ID NO: 76) |
| Surface | CTLA4 | | MACLGFORHKAQLNLATRTWPCTLLFFLLFIPVF |
| display | | | CKAMHVAQPAVVLASSRGIASFVCEYASPGKATE |
| protein | | | VRVTVLRQADSQVTEVCAATYMMGNELTFLDDS |
| (ligand) | | | ICTGTSSGNQVNLTIQGLRAMDTGLYICKVELMY |
| | | | PPPYYLGIGNGTQIYVIDPEPCPDSDFLLWILAAVS |
| | | | SGLFFYSFLLTAVSLSKMLKKRSPLTTGVYVKMP |
| | | | PTEPECEKQFQPYFIPIN (SEQ ID NO: 77) |
| Surface | CD63 | | MAVEGGMKCVKFLLYVLLLAFCACAVGLIAVGV |
| display | | | GAQLVLSQTIIQGATPGSLLPVVIIAVGVFLFLVAF |
| protein | | | VGCCGACKENYCLMITFAIFLSLIMLVEVAAAIAG |
| (multipass | | | YVFRDKVMSEFNNNFRQQMENYPKNNHTASILD |
| trans- | | | RMQADFKCCGAANYTDWEKIPSMSKNRVPDSCC |
| membrane | | | INVTVGCGINFNEKAIHKEGCVEKIGGWLRKNVL |
| protein) | | | VVAAAALGIAFVEVLGIVFACCLVKSIRSGYEVM |
| | | | (SEQ ID NO: 78) |
| Surface | CD81 | | MGVEGCTKCIKYLLFVFNFVFWLAGGVILGVAL |
| display | | | WLRHDPQTTNLLYLELGDKPAPNTFYVGIYILIAV |
| protein | | | GAVMMFVGFLGCYGAIQESQCLLGTFFTCLVILF |
| (multipass | | | ACEVAAGIWGFVNKDQIAKDVKQFYDQALQQA |
| trans- | | | VVDDDANNAKAVVKTFHETLDCCGSSTLTALTT |
| membrane | | | SVLKNNLCPSGSNIISNLFKEDCHQKIDDLFSGKL |
| protein) | | | YLIGIAAIVVAVIMIFEMILSMVLCCGIRNSSVY |
| | | | (SEQ ID NO: 79) |
| Surface | CD9 | | MPVKGGTKCIKYLLFGFNFIFWLAGIAVLAIGLW |
| display | | | LRFDSQTKSIFEQETNNNNSSFYTGVYILIGAGAL |
| protein | | | MMLVGFLGCCGAVQESQCMLGLFFGFLLVIFAIEI |
| (multipass | | | AAAIWGYSHKDEVIKEVQEFYKDTYNKLKTKDE |
| trans- | | | PQRETLKAIHYALNCCGLAGGVEQFISDICPKKDV |
| membrane | | | LETFTVKSCPDAIKEVFDNKFHIIGAVGIGIAVVMI |
| protein) | | | FGMIFSMILCCAIRRNREMV (SEQ ID NO: 80) |
| Surface | A*02:01 | | MATGSRTSLLLAFGLLCLPWLQEGSAELAGIGILT |
| display | MART1 | | VGCGASGGGGSGGGGSIQRTPKIQVYSRHPAENG |
| protein | | | KSNFLNCYVSGFHPSDIEVDLLKNGERIEKVEHSD |
| (peptide- | | | LSFSKDWSFYLLYYTEFTPTEKDEYACRVNHVTL |
| major | | | SQPKIVKWDRDMGGGGSGGGGSGGGGSGGGGS |
| histocom | | | GSHSMRYFFTSVSRPGRGEPRFIAVGYVDDTQFV |
| patibility | | | RFDSDAASQRMEPRAPWIEQEGPEYWDGETRKV |
| complex) | | | KAHSQTHRVDLGTLRGCYNQSEAGSHTVQRMYG |
| | | | CDVGSDWRFLRGYHQYAYDGKDYIALKEDLRS |
| | | | WTAADMAAQTTKHKWEAAHVAEQLRAYLEGTC |
| | | | VEWLRRYLENGKETLQRTDAPKTHMTHHAVSDH |
| | | | EATLRCWALSFYPAEITLTWQRDGEDQTQDTELV |
| | | | ETRPAGDGTFQKWAAVVVPSGQEQRYTCHVQHE |
| | | | GLPKPLTLRWEPSSQPTIPIVGIIAGLVLFGAVITGA |
| | | | VVAAVMWRRKSSDRKGGSYSQAASSDSAQGSD |
| | | | VSLTACKV (SEQ ID NO: 81) |
| Surface | A*02:01 | | MATGSRTSLLLAFGLLCLPWLQEGSASLLMWITQ |
| display | NY-ESO- | | VGCGASGGGGSGGGGSIQRTPKIQVYSRHPAENG |
| protein | 1 | | KSNFLNCYVSGFHPSDIEVDLLKNGERIEKVEHSD |
| (peptide- | | | LSFSKDWSFYLLYYTEFTPTEKDEYACRVNHVTL |
| major | | | SQPKIVKWDRDMGGGGSGGGGSGGGGSGGGGS |
| histocom | | | GSHSMRYFFTSVSRPGRGEPRFIAVGYVDDTQFV |
| patibility | | | RFDSDAASQRMEPRAPWIEQEGPEYWDGETRKV |
| complex) | | | KAHSQTHRVDLGTLRGCYNQSEAGSHTVQRMYG |
| | | | CDVGSDWRFLRGYHQYAYDGKDYIALKEDLRS |
| | | | WTAADMAAQTTKHKWEAAHVAEQLRAYLEGTC |
| | | | VEWLRRYLENGKETLQRTDAPKTHMTHHAVSDH |
| | | | EATLRCWALSFYPAEITLTWQRDGEDQTQDTELV |
| | | | ETRPAGDGTFQKWAAVVVPSGQEQRYTCHVQHE |
| | | | GLPKPLTLRWEPSSQPTIPIVGIIAGLVLFGAVITGA |
| | | | VVAAVMWRRKSSDRKGGSYSQAASSDSAQGSD |
| | | | VSLTACKV (SEQ ID NO: 82) |
| Surface | H2-Kb | | MVPCTLLLLLAAALAPTQTRASIINFEKLGCGASG |
| display | OVA | | GGGSGGGGSIQKTPQIQVYSRHPPENGKPNILNCY |
| protein | | | VTQFHPPHIEIQMLKNGKKIPKVEMSDMSFSKDW |
| (peptide- | | | SFYILAHTEFTPTETDTYACRVKHASMAEPKTVY |
| major | | | WDRDMGGGGSGGGGSGGGGSGGGGSGPHSLRY |
| histocom | | | FVTAVSRPGLGEPRYMEVGYVDDTEFVRFDSDAE |
| patibility | | | NPRYEPRARWMEQEGPEYWERETQKAKGNEQSF |
| complex) | | | RVDLRTLLGCYNQSKGGSHTIQVISGCEVGSDGR |
| | | | LLRGYQQYAYDGCDYIALNEDLKTWTAADMAA |
| | | | LITKHKWEQAGEAERLRAYLEGTCVEWLRRYLK |
| | | | NGNATLLRTDSPKAHVTHHSRPEDKVTLRCWAL |
| | | | GFYPADITLTWQLNGEELIQDMELVETRPAGDGT |
| | | | FQKWASVVVPLGKEQYYTCHVYHQGLPEPLTLR |
| | | | WEPPPSTVSNMATVAVLVVLGAAIVTGAVVAFV |
| | | | MKMRRRNTGGKGGDYALAPGSQTSDLSLPDCKV |
| | | | MVHDPHSLA (SEQ ID NO: 83) |
| Surface | CD19 | | MPPPRLLFFLLFLTPMEVRPEEPLVVKVEEGDNA |
| display | | | VLQCLKGTSDGPTQQLTWSRESPLKPFLKLSLGLP |
| protein | | | GLGIHMRPLAIWLFIFNVSQQMGGFYLCQPGPPSE |
| (receptor) | | | KAWQPGWTVNVEGSGELFRWNVSDLGGLGCGL |
| | | | KNRSSEGPSSPSGKLMSPKLYVWAKDRPEIWEGE |
| | | | PPCLPPRDSLNQSLSQDLTMAPGSTLWLSCGVPPD |
| | | | SVSRGPLSWTHVHPKGPKSLLSLELKDDRPARDM |
| | | | WVMETGLLLPRATAQDAGKYYCHRGNLTMSFH |
| | | | LEITARPVLWHWLLRTGGWKVSAVTLAYLIFCLC |
| | | | SLVGILHLQRALVLRRKRKRMTDPTRRFFKVTPPP |
| | | | GSGPQNQYGNVLSLPTPTSGLGRAQRWAAGLGG |
| | | | TAPSYGNPSSDVQADGALGSRSPPGVGPEEEEGE |
| | | | GYEEPDSEEDSEFYENDSNLGQDQLSQDGSGYEN |
| | | | PEDEPLGPEDEDSFSNAESYENEDEELTQPVARTM |
| | | | DFLSPHGSAWDPSREATSLGSQSYEDMRGILYAA |
| | | | PQLRSIRGQPGPNHEEDADSYENMDNPDGPDPA |
| | | | WGGGGRMGTWSTR (SEQ ID NO: 84) |
| Surface | CD28 | | MLRLLLALNLFPSIQVTGNKILVKQSPMLVAYDN |
| display | | | AVNLSCKYSYNLFSREFRASLHKGLDSAVEVCVV |
| protein | | | YGNYSQQLQVYSKTGFNCDGKLGNESVTFYLQN |
| (receptor) | | | LYVNQTDIYFCKIEVMYPPPYLDNEKSNGTIIHVK |
| | | | GKHLCPSPLFPGPSKPFWVLVVVGGVLACYSLLV |
| | | | TVAFIIFWVRSKRSRLLHSDYMNMTPRRPGPTRK |
| | | | HYQPYAPPRDFAAYRS (SEQ ID NO: 85) |
| Surface | PDCD1 | | MQIPQAPWPVVWAVLQLGWRPGWFLDSPDRPW |
| display | (PD1) | | NPPTFSPALLVVTEGDNATFTCSFSNTSESFVLNW |
| protein | | | YRMSPSNQTDKLAAFPEDRSQPGQDCRFRVTQLP |
| (receptor) | | | NGRDFHMSVVRARRNDSGTYLCGAISLAPKAQIK |
| | | | ESLRAELRVTERRAEVPTAHPSPSPRPAGQFQTLV |
| | | | VGVVGGLLGSLVLLVWVLAVICSRAARGTIGARR |
| | | | TGQPLKEDPSAVPVFSVDYGELDFQWREKTPEPP |
| | | | VPCVPEQTEYATIVFPSGMGTSSPARRGSADGPRS |
| | | | AQPLRPEDGHCSWPL (SEQ ID NO: 86) |
| Insulator | HS4 core | agggacagccccccccc | |
| | insulator | aaagcccccagggatgt | |
| | | aattacgtccctcccccgc | |
| | | tagggggcagcagcgag | |
| | | ccgcccggggctccgct | |
| | | ccggtccggcgctcccc | |
| | | ccgcatccccgagccgg | |
| | | cagcgtgcggggacagc | |
| | | ccgggcacggggaaggt | |
| | | ggcacgggatcgctttcc | |
| | | tctgaacgcttctcgctgc | |
| | | tctttgagcctgcagacac | |
| | | ctggggggatacgggga | |
| | | aaa (SEQ ID NO: | |
| | | 87) | |

1. A nucleic acid sequence comprising:
(a) a first expressible nucleic acid and a second expressible nucleic acid;
(b) a first regulatory sequence operably linked to the first expressible nucleic acid;
(c) a second regulatory sequence operably linked to the second expressible nucleic acid; and
(d) a serine recombinase element encoding a serine recombinase positioned on either the first or the second expressible nucleic acid;
wherein (a), (b), (c) and (d) are positioned between a 5′ viral packaging sequence and a 3′ viral packaging sequence.

2. The nucleic acid of claim 1, wherein the serine recombinase is a large serine recombinase or functional variant thereof; wherein the first expressible nucleic acid comprises a first recombinase attachment element; and wherein the second expressible nucleic acid comprises a second recombinase attachment element.

3. The nucleic acid of either of claim 1, wherein the 5′ viral packaging sequence and the 3′ viral packaging sequence is a 5′ LTR and a 3′ LTR, respectively.

4. The nucleic acid of claim 3, wherein the serine recombinase element comprises SEQ ID NO: 13 or a functional variant thereof comprising at least about 75% sequence identity to SEQ ID NO:13.

5. The nucleic acid of claim 3, wherein the first recombinase attachment element comprises SEQ ID NO:5 or a functional variant thereof comprising at least about 75% sequence identity to SEQ ID NO:5; and wherein the second recombinase element comprises SEQ ID NO:6 or a functional variant thereof comprising at least about 75% sequence identity to SEQ ID NO:6.

6. The nucleic acid sequence of claim 1 further comprising (e) a first antibiotic selection nucleic acid sequence positioned on the first or second expressible nucleic acid sequence.

7. The nucleic acid sequence of claim 1, wherein the 5′ LTR is a 5′ lentiviral LTR and the 3′ LTR is a 3′ lentiviral LTR.

8. The nucleic acid sequence of claim 1,
wherein the 5′ LTR comprises SEQ ID NO:17 or a functional variant comprising about 75% sequence identity to SEQ ID NO:17; and
wherein the 3′ LTR comprises SEQ ID NO:18 or a functional variant comprising about 75% sequence identity to SEQ ID NO:18.

9. The nucleic acid sequence of claim 1 further comprising post-transcriptional regulatory element positioned on the first or second expressible nucleic acid sequences.

10. The nucleic acid sequence of claim 1, wherein the first expressible nucleic acid sequence and/or the second expressible nucleic acid sequence comprise a cellular tag.

11. The nucleic acid sequence of claim 1, wherein one or both of the first regulatory sequence or the second regulatory sequence comprises one or a combination of nucleic acid sequence chosen from:
(x) SEQ ID NO:2 or a functional variant of SEQ ID NO:2 that comprises at least about 75% sequence identity to SEQ ID NO:2;
(y) SEQ ID NO:3 or a functional variant of SEQ ID NO:3 that comprises at least about 75% sequence identity to SEQ ID NO:3; and
(z) SEQ ID NO:4 or a functional variant of SEQ ID NO:4 that comprises at least about 75% sequence identity to SEQ ID NO:4.

12. A nucleic acid molecule comprising the nucleic acid sequence of claim 1.

13. A cell or cell line comprising the nucleic acid sequence of claim 1.

14. The cell or cell line of claim 13, wherein the cell is a 293T cell or cell line.

15. A kit comprising:
(a) the cell or cell line of any of claim 13;
(b) instructions for growing the cell or cell line of (a).

16. The kit of claim 15 further comprising a first container, the container comprising a nucleic acid molecule comprising a payload positioned between a first and second recombinase attachment element.

17. The kit of claim 16 further comprising a second container comprising a nucleic acid molecule comprising one or a plurality of viral proteins that associate with the 5′ LTR and the 3′ LTR.

18. A composition comprising:
a nucleic acid sequence comprising
(a) a first expressible nucleic acid and a second expressible nucleic acid;
(b) a first regulatory sequence operably linked to the first expressible nucleic acid;
(c) a second regulatory sequence operably linked to the second expressible nucleic acid; and
(d) a serine recombinase element encoding a serine recombinase positioned on either the first or the second expressible nucleic acid; wherein (a), (b), (c) and (d) are positioned between a 5′ viral LTR and a 3′ viral LTR; or
a cell comprising the nucleic acid sequence.

19. The composition of claim 18 further comprising one or a plurality of retroviruses, wherein the retrovirus comprises the nucleic acid sequence.

20. A library of viral particles comprising one of a plurality of homogenous viral particles comprising the nucleic acid sequences of claim 1.

21. A method of generating a library of homogenous viral particles comprising:
(a) exposing the cell or cell line of claim 13 to a cell culture medium under condition sufficient to grow the cell or cell line.

22. The method claim 21 further comprising:
(b) exposing the cell or cell line to one or a plurality of nucleic acid molecules comprising a payload positioned between a first and a second recombinase attachment element.

23. The method of claim 22 further comprising a step (c) allowing a time period sufficient to enable recombination between the nucleic acid molecule comprising the payload and the nucleic acid sequence of any of claims 1 through 23, such that the payload is exchanged for a region of the nucleic acid sequence between the first recombinase attachment element and the second recombinase attachment element.

24. The method of claim 23 further comprising a step (d) transfecting the cell or cell line with a nucleic acid molecule comprising a nucleic acid sequence encoding a viral packing protein after step (a), (b) and (c).

25. The method of claim 24 further comprising a step (e) culturing the cell or cell line for a time period sufficient for the cell or cell line to produce a viral particle comprising the viral packaging protein encapsulating one or more nucleic acid sequences comprising the payload.

26. The method of claim 25, wherein the time period sufficient for the cell or cell line to produce a viral particle comprising the viral packaging protein encapsulating one or more nucleic acid sequences comprising the payload is from about 0.5 days to about 2 days.
